(19) United States
(12) Patent Application Publication
(10) Pub. No.: US 2022/0296703 A1
(43) Pub. Date: Sep. 22, 2022

(54) COBMINATION OF HEPATITIS B VIRUS (HBV) VACCINES AND ANTI-PD-1 OR ANTI-PD-L1 ANTIBODY

(71) Applicant: Janssen Sciences Ireland Unlimited Company, Co Cork (IE)

(72) Inventors: Helen HORTON, Beerse (BE); An Martine M. DE CREUS, Beerse (BE); Ellen Rosalie A. VAN GULCK, Beerse (BE)

(21) Appl. No.: 17/596,787

(22) PCT Filed: Jun. 18, 2020
(86) PCT No.: PCT/IB2020/055701

§ 371 Date: Dec. 17, 2021

Related U.S. Application Data
(60) Provisional application No. 62/862,791, filed on Jun. 18, 2019.

(51) Int. Cl. A61K 39/29; A61K 39/395; A61P 31/20; A61P 37/04

(52) U.S. Cl. CPC A61K 39/292; A61K 39/3955; A61P 31/20; A61P 37/04; A61K 2039/53

(57) ABSTRACT

Therapeutic combinations of hepatitis B virus (HBV) vaccines and an anti-PD-1 or anti-PD-L1 antibody or antigen-binding fragment thereof are described. Methods of inducing an immune response against HBV or treating an HBV-induced disease, particularly in individuals having chronic HBV infection, using the disclosed therapeutic combinations are also described.

### CROSS REFERENCE TO RELATED APPLICATION

This application claims priority to U.S. Provisional Application No. 62/862,791 filed on Jun. 18, 2019, the disclosure of which is incorporated herein by reference in its entirety.

### REFERENCE TO SEQUENCE LISTING SUBMITTED ELECTRONICALLY

This application contains a sequence listing, which is submitted electronically via EFS-Web as an ASCII formatted sequence listing with a file name “065814_15WO1_Sequence_Listing” and a creation date of Jun. 15, 2020 and having a size of 46 kb. The sequence listing submitted via EFS-Web is part of the specification and is herein incorporated by reference in its entirety.

### BACKGROUND OF THE INVENTION

Hepatitis B virus (HBV) is a small 3.2-kb hepatotropic DNA virus that encodes four open reading frames and seven proteins. Approximately 240 million people have chronic hepatitis B infection (chronic HBV), characterized by persistent virus and subvirus particles in the blood for more than 6 months (Cohen et al. J. Viral Hepat. (2011) 18(6), 377-83). Persistent HBV infection leads to T-cell exhaustion in circulating and intrahepatic HBV-specific CD4+ and CD8+ T-cells through chronic stimulation of HBV-specific T-cell receptors with viral peptides and circulating antigens. As a result, T-cell polyfunctionality is decreased (i.e., decreased levels of IL-2, tumor necrosis factor (TNF)-α, IFN-γ, and lack of proliferation).

A safe and effective prophylactic vaccine against HBV infection has been available since the 1980s and is the mainstay of hepatitis B prevention (World Health Organization, Hepatitis B: Fact sheet No. 204 [Internet] 2015 March.). The World Health Organization recommends vaccination of all infants, and, in countries where there is low or intermediate hepatitis B endemicity, vaccination of all children and adolescents (<18 years of age), and of people of certain at risk population categories. Due to vaccination, worldwide infection rates have dropped dramatically. However, prophylactic vaccines do not cure established HBV infection.

Chronic HBV is currently treated with IFN-α and nucleoside or nucleotide analogs, but there is no ultimate cure due to the persistence in infected hepatocytes of an intracellular viral replication intermediate called covalently closed circular DNA (cccDNA), which plays a fundamental role as a template for viral RNAs, and thus new virions. It is thought that induced virus-specific T-cell and B-cell responses can effectively eliminate cccDNA-carrying hepatocytes. Current therapies targeting the HBV polymerase suppress viremia, but offer limited effect on cccDNA that resides in the nucleus and related production of circulating antigen. The most rigorous form of a cure may be elimination of HBV cccDNA from the organism, which has neither been observed as a naturally occurring outcome nor as a result of any therapeutic intervention. However, loss of HBV surface antigens (HBsAg) is a clinically credible equivalent of a cure, since disease relapse can occur only in cases of severe immunosuppression, which can then be prevented by prophylactic treatment. Thus, at least from a clinical standpoint, loss of HBsAg is associated with the most stringent form of immune reconstitution against HBV.

For example, immune modulation with pegylated interferon (pegIFN)-α has proven better in comparison to nucleoside or nucleotide therapy in terms of sustained off-treatment response with a finite treatment course. Besides a direct antiviral effect, IFN-α is reported to exert epigenetic suppression of cccDNA in cell culture and humanized mice, which leads to reduction of virion productivity and transcripts (Belloni et al. J. Clin. Invest. (2012) 122(2), 529-537). However, this therapy is still fraught with side-effects and overall responses are rather low, in part because IFN-α has only poor modulatory influences on HBV-specific T-cells. In particular, cure rates are low (<10%) and toxicity is high. Likewise, direct acting HBV antivirals, namely the HBV polymerase inhibitors entecavir and tenofovir, are effective as monotherapy in inducing viral suppression with a high genetic barrier to emergence of drug resistant mutants and consecutive prevention of liver disease progression. However, cure of chronic hepatitis B, defined by HBsAg loss or seroconversion, is rarely achieved with such HBV polymerase inhibitors. Therefore, these antivirals in theory need to be administered indefinitely to prevent reoccurrence of liver disease, similar to antiretroviral therapy for human immunodeficiency virus (HIV).

Therapeutic vaccination has the potential to eliminate HBV from chronically infected patients (Michel et al. J. Hepatol. (2011) 54(6), 1286-1296). Many strategies have been explored, but to date therapeutic vaccination has not proven successful.

### BRIEF SUMMARY OF THE INVENTION

Accordingly, there is an unmet medical need in the treatment of hepatitis B virus (HBV), particularly chronic HBV, for a finite well-tolerated treatment with a higher cure rate. The invention satisfies this need by providing therapeutic combinations or compositions and methods for inducing an immune response against hepatitis B viruses (HBV) infection. The immunogenic compositions/combinations and methods of the invention can be used to provide therapeutic immunity to a subject, such as a subject having chronic HBV infection.

In a general aspect, the application relates to therapeutic combinations or compositions comprising one or more HBV antigens, or one or more polynucleotides encoding the HBV antigens, and an anti-PD-1 or anti-PD-L1 antibody or antigen-binding fragment thereof, for use in treating an HBV infection in a subject in need thereof.

In one embodiment, the therapeutic combination comprises:

i) at least one of:
a) a truncated HBV core antigen consisting of an amino acid sequence that is at least 95%, such as at least 95%, 96%, 97%, 98%, 99% or 100%, identical to SEQ ID NO: 2,
b) a first non-naturally occurring nucleic acid molecule comprising a first polynucleotide sequence encoding the truncated HBV core antigen;
c) an HBV polymerase antigen having an amino acid sequence that is at least 90%, such as at least 90%, 91%, 92%, 93%, 94%, 95%, 96%, 97%, 98%, 99% or 100%, identical to SEQ ID NO: 7, wherein the HBV polymerase antigen does not have reverse transcriptase activity and RNase H activity, and
d) a second non-naturally occurring nucleic acid molecule comprising a second polynucleotide sequence encoding the HBV polymerase antigen; and
   ii) an anti-PD-1 or anti-PD-L1 antibody or antigen-binding fragment thereof, such as those described herein.

In one embodiment, the truncated HBV core antigen consists of the amino acid sequence of SEQ ID NO: 2 or SEQ ID NO: 4, and the HBV polymerase antigen comprises the amino acid sequence of SEQ ID NO: 7.

In one embodiment, the therapeutic combination comprises at least one of the HBV polymerase antigen and the truncated HBV core antigen. In certain embodiments, the therapeutic combination comprises the HBV polymerase antigen and the truncated HBV core antigen.

In one embodiment, the therapeutic combination comprises at least one of the first non-naturally occurring nucleic acid molecule comprising the first polynucleotide sequence encoding the truncated HBV core antigen, and the second non-naturally occurring nucleic acid molecule comprising the second polynucleotide sequence encoding the HBV polymerase antigen. In certain embodiments, the first non-naturally occurring nucleic acid molecule further comprises a polynucleotide sequence encoding a signal sequence operably linked to the N-terminus of the truncated HBV core antigen, and the second non-naturally occurring nucleic acid molecule further comprises a polynucleotide sequence encoding a signal sequence operably linked to the N-terminus of the HBV polymerase antigen, preferably, the signal sequence independently comprises the amino acid sequence of SEQ ID NO: 9 or SEQ ID NO: 15, more preferably, the signal sequence is encoded by the polynucleotide sequence of SEQ ID NO: 8 or SEQ ID NO: 14, respectively.

In certain embodiments, the first polynucleotide sequence comprises the polynucleotide sequence having at least 90%, such as at least 90%, 91%, 92%, 93%, 94%, 95%, 96%, 97%, 98%, 99% or 100%, sequence identity to SEQ ID NO: 1 or SEQ ID NO: 3.

In certain embodiments, the second polynucleotide sequence comprises a polynucleotide sequence having at least 90%, such as at least 90%, 91%, 92%, 93%, 94%, 95%, 96%, 97%, 98%, 99% or 100%, sequence identity to SEQ ID NO: 5 or SEQ ID NO: 6.

In an embodiment, a therapeutic combination comprises:
a) a first non-naturally occurring nucleic acid molecule comprising a first polynucleotide sequence encoding a truncated HBV core antigen consisting of an amino acid sequence that is at least 95%, such as at least 95%, 96%, 97%, 98%, 99% or 100%, identical to SEQ ID NO: 2;
b) a second non-naturally occurring nucleic acid molecule comprising a second polynucleotide sequence encoding an HBV polymerase antigen having an amino acid sequence that is at least 90%, such as at least 90%, 91%, 92%, 93%, 94%, 95%, 96%, 97%, 98%, 99% or 100%, identical to SEQ ID NO: 7, wherein the HBV polymerase antigen does not have reverse transcriptase activity and RNase H activity; and
c) an anti-PD-1 or anti-PD-L1 antibody or antigen-binding fragment thereof, preferably selected from the group consisting of:
   i) 17D8, 2D3, 4H1, 5C4, 4A11, 7D3, 5F4, H409A11, hPD-1.08A, hPD-1.09A, 109A-H, K09A-L-11, K09A-L-16, K09A-L-17, 409A-H, H1M7789N, H1M7799N, H1M7800N, H2M7780N, H2M7788N, H2M7790N, H2M7791N, H2M7794N, H2M7795N, H2M7796N, H2M7798N, H4H9019P, H4xH9034P2, H4xH9035P2, H4xH9037P2, H4xH9045P2, H4xH9048P2, H4H9057P2, H4H9068P2, H4xH9119P2, H4xH9120P2, H4xH9128P2, H4xH9135P2, H4xH9145P2, H4xH8992P, H4xH8999P, H4xH9008P, H4H7798N, H4H7795N2, H4H9008P, H4H9048P2, PD1-0103, PD1-0098, PD1-0050, PD1-0069, PD1-0073, PD1-0078, PD1-0102, PD1-0103_01, PD1-0103_02, PD1-0103_03, PD1-0103_04, PD1-0103-0312, PD1-0103-0313, PD1-0103-0314, PD1-0103-0315, 1.7.3 hAb, 1.49.9 hAb, 1.103.11 hAb, 1.103.11-v2 hAb, 1.139.15 hAb, 1.153.7 hAb, and variants thereof,
   ii) an antibody binding to an epitope having a sequence of SEHSI, DPFEL, KLNG, QTSWK, LHFEP, NDNGSY, TTLYVT, or LAAFPEDRSQPGQDCR; and
   iii) Nivolumab (MDX-1 106, Opdivo; Bristol-Myers Squibb), Pembrolizumab (MK-3475, Keytruda, lambrolizumab, BMS-936558; Merck), TSR-042 (Tesaro, Inc.), REGN2810 (Regeneron Pharmaceuticals), EH12.2H7 (BioLegend, catalog no. 329902), Avelumab (Bavencio; EMD Serono, Pfizer), Durvalumab (Imfinzi, AstraZeneca), Cemiplimab (REGN-2810, Libtayo; Regeneron), BMS-936559, Atezolizumab (Tecentriq, Genentech), or an equivalent thereto.

Preferably, the therapeutic combination comprises a) a first non-naturally occurring nucleic acid molecule comprising a first polynucleotide sequence encoding an truncated HBV core antigen consisting of the amino acid sequence of SEQ ID NO: 2 or SEQ ID NO: 4; b) a second non-naturally occurring nucleic acid molecule comprising a second polynucleotide sequence encoding an HBV polymerase antigen having the amino acid sequence of SEQ ID NO: 7, and (c) an anti-PD-1 or anti-PD-L1 antibody or antigen-binding fragment thereof.

Preferably, the therapeutic combination comprises a first non-naturally occurring nucleic acid molecule comprising a polynucleotide sequence having at least 90%, such as at least 90%, 91%, 92%, 93%, 94%, 95%, 96%, 97%, 98%, 99% or 100%, sequence identity to SEQ ID NO: 1 or SEQ ID NO: 3, and a second non-naturally occurring nucleic acid molecule comprising the polynucleotide sequence having at least 90%, such as at least 90%, 91%, 92%, 93%, 94%, 95%, 96%, 97%, 98%, 99% or 100%, sequence identity to SEQ ID NO: 5 or SEQ ID NO: 6.

More preferably, the therapeutic combination comprises a) a first non-naturally occurring nucleic acid molecule comprising a first polynucleotide sequence of SEQ ID NO: 1 or SEQ ID NO: 3; b) a second non-naturally occurring nucleic acid molecule comprising a second polynucleotide sequence of SEQ ID NO: 5 or 6; and c) an anti-PD-1 or anti-PD-L1 antibody or antigen-binding fragment thereof.

In an embodiment, each of the first and the second non-naturally occurring nucleic acid molecules is a DNA molecule, preferably the DNA molecule is present on a plasmid or a viral vector.

In another embodiment, each of the first and the second non-naturally occurring nucleic acid molecules is an RNA molecule, preferably an mRNA or a self-replicating RNA molecule.

In some embodiments, each of the first and the second non-naturally occurring nucleic acid molecules is independently formulated with a lipid nanoparticle (LNP).

In another general aspect, the application relates to a kit comprising a therapeutic combination of the application.

The application also relates to a therapeutic combination or kit of the application for use in inducing an immune response against hepatitis B virus (HBV); and use of a therapeutic combination, composition or kit of the application in the manufacture of a medicament for inducing an immune response against hepatitis B virus (HBV). The use can further comprise a combination with another immunogenic or therapeutic agent, preferably another HBV antigen or another HBV therapy. Preferably, the subject has chronic HBV infection.

The application further relates to a therapeutic combination or kit of the application for use in treating an HBV-induced disease in a subject in need thereof, and use of a therapeutic combination or kit of the application in the manufacture of a medicament for treating an HBV-induced disease in a subject in need thereof. The use can further comprise a combination with another therapeutic agent, preferably another anti-HBV antigen. Preferably, the subject has chronic HBV infection, and the HBV-induced disease is selected from the group consisting of advanced fibrosis, cirrhosis, and hepatocellular carcinoma (HCC).

The application also relates to a method of inducing an immune response against an HBV or a method of treating an HBV infection or an HBV-induced disease, comprising administering to a subject in need thereof a therapeutic combination according to embodiments of the invention.

Other aspects, features and advantages of the invention will be apparent from the following disclosure, including the detailed description of the invention and its preferred embodiments and the appended claims.

### BRIEF DESCRIPTION OF THE DRAWINGS

The foregoing summary, as well as the following detailed description of preferred embodiments of the present application, will be better understood when read in conjunction with the appended drawings. It should be understood, however, that the application is not limited to the precise embodiments shown in the drawings.

FIG. 1A and FIG. 1B show schematic representations of DNA plasmids according to embodiments of the application; FIG. 1A shows a DNA plasmid encoding an HBV core antigen according to an embodiment of the application; FIG. 1B shows a DNA plasmid encoding an HBV polymerase (pol) antigen according to an embodiment of the application; the HBV core and pol antigens are expressed under control of a CMV promoter with an N-terminal cystatin S signal peptide that is cleaved from the expressed antigen upon secretion from the cell; transcriptional regulatory elements of the plasmid include an enhancer sequence located between the CMV promoter and the polynucleotide sequence encoding the HBV antigen and a bGH polyadenylation sequence located downstream of the polynucleotide sequence encoding the HBV antigen; a second expression cassette is included in the plasmid in reverse orientation including a kanamycin resistance gene under control of an Ampr (bla) promoter; an origin of replication (pUC) is also included in reverse orientation.

FIG. 2A and FIG. 2B. show the schematic representations of the expression cassettes in adenoviral vectors according to embodiments of the application; FIG. 2A shows the expression cassette for a truncated HBV core antigen, which contains a CMV promoter, an intron (a fragment derived from the human ApoAI gene—GenBank accession X01038 base pairs 295-523, harboring the ApoAI second intron), a human immunoglobulin secretion signal, followed by a coding sequence for a truncated HBV core antigen and a SV40 polyadenylation signal; FIG. 2B shows the expression cassette for a fusion protein of a truncated HBV core antigen operably linked to an HBV polymerase antigen, which is otherwise identical to the expression cassette for the truncated HBV core antigen except the HBV antigen.

FIG. 3 shows ELISPOT responses of Balb/c mice immunized with different DNA plasmids expressing HBV core antigen or HBV pol antigen, as described in Example 3; peptide pools used to stimulate splenocytes isolated from the various vaccinated animal groups are indicated in gray scale; the number of responsive T-cells are indicated on the y-axis expressed as spot forming cells (SFC) per 10⁶ splenocytes;

FIG. 4A and FIG. 4B show immune responses measured by ELISPOT of mice that were administered DNA plasmids according to embodiments of the application (at d0 and d21) along with anti-PD-1 or isotype antibodies according to embodiments of the application; FIG. 4A shows results for mice that received 3 injections of anti-PD-1 (black) or isotype (grey) starting 1 week after the first vaccination (i.e., d7, d14, d21); FIG. 4B shows results for the mice that were treated with anti-PD-1 (black) or isotype (grey) at the same time of the first vaccination and then every 7 days (i.e., d0, d7, d14, d21); and

FIG. 5A, FIG. 5B, FIG. 5C, FIG. 5D, FIG. 5E and FIG. 5F show immune responses measured by ICS of splenocytes that were isolated from mice that were administered DNA plasmids according to embodiments of the application (at d0 and d21) along with anti-PD-1 or isotype antibodies according to embodiments of the application, with results represented as mean+/−SEM; the splenocytes were stimulated with core (FIG. 5A and FIG. 5D), pol1 (FIG. 5B and FIG. 5E) or pol2 (FIG. 5C and FIG. 5F) for 6 hours, and responses against IFN-γ, TL-2 or TNF-α were then measured; FIG. 5A, FIG. 5B and FIG. 5C show results for splenocytes isolated from mice that received 3 injections of anti-PD-1 (black) or isotype (grey) starting 1 week after the first vaccination (i.e., d7, d14, d21); FIG. 5D, FIG. 5E and FIG. 5F show results for the mice that were treated with anti-PD-1 (black) or isotype (grey) at the same time of the first vaccination and then every 7 days (i.e., d0, d7, d14, d21).

FIG. 6 shows the proliferative capacity of CD8 T-cells in intrahepatic lymphocytes (IHL) that were isolated from mice that were administered DNA plasmids according to embodiments of the application (at d28 and d49) along with anti-PD-1 or isotype antibodies according to embodiments of the application, with results represented as mean+/−SEM.

FIG. 7 shows the proliferative capacity of CD8 T-cells in splenocytes that were isolated from mice that were administered DNA plasmids according to embodiments of the application (at d28 and d49) along with anti-PD-1 or isotype antibodies according to embodiments of the application, with results represented as mean+/−SEM.

### DETAILED DESCRIPTION OF THE INVENTION

Various publications, articles and patents are cited or described in the background and throughout the specification; each of these references is herein incorporated by reference in its entirety. Discussion of documents, acts, materials, devices, articles or the like which has been included in the present specification is for the purpose of providing context for the invention. Such discussion is not an admission that any or all of these matters form part of the prior art with respect to any inventions disclosed or claimed.

Unless defined otherwise, all technical and scientific terms used herein have the same meaning as commonly understood to one of ordinary skill in the art to which this invention pertains. Otherwise, certain terms used herein have the meanings as set forth in the specification. All patents, published patent applications and publications cited herein are incorporated by reference as if set forth fully herein.

It must be noted that as used herein and in the appended claims, the singular forms “a,” “an,” and “the” include plural reference unless the context clearly dictates otherwise.

Unless otherwise indicated, the term “at least” preceding a series of elements is to be understood to refer to every element in the series. Those skilled in the art will recognize, or be able to ascertain using no more than routine experimentation, many equivalents to the specific embodiments of the invention described herein. Such equivalents are intended to be encompassed by the invention.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps. When used herein the term “comprising” can be substituted with the term “containing” or “including” or sometimes when used herein with the term “having”.

When used herein “consisting of” excludes any element, step, or ingredient not specified in the claim element. When used herein, “consisting essentially of” does not exclude materials or steps that do not materially affect the basic and novel characteristics of the claim. Any of the aforementioned terms of “comprising”, “containing”, “including”, and “having”, whenever used herein in the context of an aspect or embodiment of the application can be replaced with the term “consisting of” or “consisting essentially of” to vary scopes of the disclosure.

As used herein, the conjunctive term “and/of” between multiple recited elements is understood as encompassing both individual and combined options. For instance, where two elements are conjoined by “and/or,” a first option refers to the applicability of the first element without the second. A second option refers to the applicability of the second element without the first. A third option refers to the applicability of the first and second elements together. Any one of these options is understood to fall within the meaning, and therefore satisfy the requirement of the term “and/or” as used herein. Concurrent applicability of more than one of the options is also understood to fall within the meaning, and therefore satisfy the requirement of the term “and/or.”

Unless otherwise stated, any numerical value, such as a concentration or a concentration range described herein, are to be understood as being modified in all instances by the term “about.” Thus, a numerical value typically includes ±10% of the recited value. For example, a concentration of 1 mg/mL includes 0.9 mg/mL to 1.1 mg/mL. Likewise, a concentration range of 1 mg/mL to 10 mg/mL includes 0.9 mg/mL to 11 mg/mL. As used herein, the use of a numerical range expressly includes all possible subranges, all individual numerical values within that range, including integers within such ranges and fractions of the values unless the context clearly indicates otherwise.

The phrases “percent (%) sequence identity” or “% identity” or “% identical to” when used with reference to an amino acid sequence describe the number of matches (“hits”) of identical amino acids of two or more aligned amino acid sequences as compared to the number of amino acid residues making up the overall length of the amino acid sequences. In other terms, using an alignment, for two or more sequences the percentage of amino acid residues that are the same (e.g. 90%, 91%, 92%, 93%, 94%, 95%, 97%, 98%, 99%, or 100% identity over the full-length of the amino acid sequences) may be determined, when the sequences are compared and aligned for maximum correspondence as measured using a sequence comparison algorithm as known in the art, or when manually aligned and visually inspected. The sequences which are compared to determine sequence identity may thus differ by substitution(s), addition(s) or deletion(s) of amino acids. Suitable programs for aligning protein sequences are known to the skilled person. The percentage sequence identity of protein sequences can, for example, be determined with programs such as CLUSTALW, Clustal Omega, FASTA or BLAST, e.g. using the NCBI BLAST algorithm (Altschul S F, et al (1997), Nucleic Acids Res. 25:3389-3402).

As used herein, the terms and phrases “in combination,” “in combination with,” “co-delivery,” and “administered together with” in the context of the administration of two or more therapies or components to a subject refers to simultaneous administration or subsequent administration of two or more therapies or components, such as two vectors, e.g., DNA plasmids, peptides, or a therapeutic combination and an adjuvant. “Simultaneous administration” can be administration of the two or more therapies or components at least within the same day. When two components are “administered together with” or “administered in combination with,” they can be administered in separate compositions sequentially within a short time period, such as 24, 20, 16, 12, 8 or 4 hours, or within 1 hour, or they can be administered in a single composition at the same time. “Subsequent administration” can be administration of the two or more therapies or components in the same day or on separate days. The use of the term “in combination with” does not restrict the order in which therapies or components are administered to a subject. For example, a first therapy or component (e.g. first DNA plasmid encoding an HBV antigen) can be administered prior to (e.g., 5 minutes to one hour before), concomitantly with or simultaneously with, or subsequent to (e.g., 5 minutes to one hour after) the administration of a second therapy or component (e.g., second DNA plasmid encoding an HBV antigen), and/or a third therapy or component (e.g., anti-PD-1 or anti-PD-L1 antibody or antigen-binding fragment thereof). In some embodiments, a first therapy or component (e.g. first DNA plasmid encoding an HBV antigen), a second therapy or component (e.g., second DNA plasmid encoding an HBV antigen), and a third therapy or component (e.g., anti-PD-1 or anti-PD-L1 antibody or antigen-binding fragment thereof) are administered in the same composition. In other embodiments, a first therapy or component (e.g. first DNA plasmid encoding an HBV antigen), a second therapy or component (e.g., second DNA plasmid encoding an HBV antigen), and a third therapy or component (e.g., anti-PD-1 or anti-PD-L1 antibody or antigen-binding fragment thereof) are administered in separate compositions, such as two or three separate compositions.

As used herein, a “non-naturally occurring” nucleic acid or polypeptide, refers to a nucleic acid or polypeptide that does not occur in nature. A “non-naturally occurring” nucleic acid or polypeptide can be synthesized, treated, fabricated, and/or otherwise manipulated in a laboratory and/or manufacturing setting. In some cases, a non-naturally occurring nucleic acid or polypeptide can comprise a naturally-occurring nucleic acid or polypeptide that is treated, processed, or manipulated to exhibit properties that were not present in the naturally-occurring nucleic acid or polypeptide, prior to treatment. As used herein, a “non-naturally occurring” nucleic acid or polypeptide can be a nucleic acid or polypeptide isolated or separated from the natural source in which it was discovered, and it lacks covalent bonds to sequences with which it was associated in the natural source. A “non-naturally occurring” nucleic acid or polypeptide can be made recombinantly or via other methods, such as chemical synthesis.

As used herein, “subject” means any animal, preferably a mammal, most preferably a human, to whom will be or has been treated by a method according to an embodiment of the application. The term “mammal” as used herein, encompasses any mammal. Examples of mammals include, but are not limited to, cows, horses, sheep, pigs, cats, dogs, mice, rats, rabbits, guinea pigs, non-human primates (NHPs) such as monkeys or apes, humans, etc., more preferably a human.

As used herein, the term “operably linked” refers to a linkage or a juxtaposition wherein the components so described are in a relationship permitting them to function in their intended manner. For example, a regulatory sequence operably linked to a nucleic acid sequence of interest is capable of directing the transcription of the nucleic acid sequence of interest, or a signal sequence operably linked to an amino acid sequence of interest is capable of secreting or translocating the amino acid sequence of interest over a membrane.

In an attempt to help the reader of the application, the description has been separated in various paragraphs or sections, or is directed to various embodiments of the application. These separations should not be considered as disconnecting the substance of a paragraph or section or embodiments from the substance of another paragraph or section or embodiments. To the contrary, one skilled in the art will understand that the description has broad application and encompasses all the combinations of the various sections, paragraphs and sentences that can be contemplated. The discussion of any embodiment is meant only to be exemplary and is not intended to suggest that the scope of the disclosure, including the claims, is limited to these examples. For example, while embodiments of HBV vectors of the application (e.g., plasmid DNA or viral vectors) described herein may contain particular components, including, but not limited to, certain promoter sequences, enhancer or regulatory sequences, signal peptides, coding sequence of an HBV antigen, polyadenylation signal sequences, etc. arranged in a particular order, those having ordinary skill in the art will appreciate that the concepts disclosed herein may equally apply to other components arranged in other orders that can be used in HBV vectors of the application. The application contemplates use of any of the applicable components in any combination having any sequence that can be used in HBV vectors of the application, whether or not a particular combination is expressly described. The invention generally relates to a therapeutic combination comprising one or more HBV antigens and at least one anti-PD-1 or anti-PD-L1 antibody or antigen-binding fragment thereof.

### Hepatitis B Virus (HBV)

As used herein “hepatitis B virus” or “HBV” refers to a virus of the hepadnaviridae family. HBV is a small (e.g., 3.2 kb) hepatotropic DNA virus that encodes four open reading frames and seven proteins. The seven proteins encoded by HBV include small (S), medium (M), and large (L) surface antigen (HBsAg) or envelope (Env) proteins, pre-Core protein, core protein, viral polymerase (Pol), and HBx protein. HBV expresses three surface antigens, or envelope proteins, L, M, and S, with S being the smallest and L being the largest. The extra domains in the M and L proteins are named Pre-S2 and Pre-S1, respectively. Core protein is the subunit of the viral nucleocapsid. Pol is needed for synthesis of viral DNA (reverse transcriptase, RNaseH, and primer), which takes place in nucleocapsids localized to the cytoplasm of infected hepatocytes. PreCore is the core protein with an N-terminal signal peptide and is proteolytically processed at its N and C termini before secretion from infected cells, as the so-called hepatitis B e-antigen (HBeAg). HBx protein is required for efficient transcription of covalently closed circular DNA (cccDNA). HBx is not a viral structural protein. All viral proteins of HBV have their own mRNA except for core and polymerase, which share an mRNA. With the exception of the protein pre-Core, none of the HBV viral proteins are subject to post-translational proteolytic processing.

The HBV virion contains a viral envelope, nucleocapsid, and single copy of the partially double-stranded DNA genome. The nucleocapsid comprises 120 dimers of core protein and is covered by a capsid membrane embedded with the S, M, and L viral envelope or surface antigen proteins. After entry into the cell, the virus is uncoated and the capsid-containing relaxed circular DNA (rcDNA) with covalently bound viral polymerase migrates to the nucleus. During that process, phosphorylation of the core protein induces structural changes, exposing a nuclear localization signal enabling interaction of the capsid with so-called importins. These importins mediate binding of the core protein to nuclear pore complexes upon which the capsid disassembles and polymerase/rcDNA complex is released into the nucleus. Within the nucleus the rcDNA becomes deproteinized (removal of polymerase) and is converted by host DNA repair machinery to a covalently closed circular DNA (cccDNA) genome from which overlapping transcripts encode for HBeAg, HBsAg, Core protein, viral polymerase and HBx protein. Core protein, viral polymerase, and pre-genomic RNA (pgRNA) associate in the cytoplasm and self-assemble into immature pgRNA-containing capsid particles, which further convert into mature rcDNA-capsids and function as a common intermediate that is either enveloped and secreted as infectious virus particles or transported back to the nucleus to replenish and maintain a stable cccDNA pool.

To date, HBV is divided into four serotypes (adr, adw, ayr, ayw) based on antigenic epitopes present on the envelope proteins, and into eight genotypes (A, B, C, D, E, F, G, and H) based on the sequence of the viral genome. The HBV genotypes are distributed over different geographic regions. For example, the most prevalent genotypes in Asia are genotypes B and C. Genotype D is dominant in Africa, the Middle East, and India, whereas genotype A is widespread in Northern Europe, sub-Saharan Africa, and West Africa.

HBV Antigens

As used herein, the terms “HBV antigen,” “antigenic polypeptide of HBV,” “HBV antigenic polypeptide,” “HBV antigenic protein,” “HBV immunogenic polypeptide,” and “HBV immunogen” all refer to a polypeptide capable of inducing an immune response, e.g., a humoral and/or cellular mediated response, against an HBV in a subject. The HBV antigen can be a polypeptide of HBV, a fragment or epitope thereof, or a combination of multiple HBV polypeptides, portions or derivatives thereof Δn HBV antigen is capable of raising in a host a protective immune response, e.g., inducing an immune response against a viral disease or infection, and/or producing an immunity (i.e., vaccinates) in a subject against a viral disease or infection, that protects the subject against the viral disease or infection. For example, an HBV antigen can comprise a polypeptide or immunogenic fragment(s) thereof from any HBV protein, such as HBeAg, pre-core protein, HBsAg (S, M, or L proteins), core protein, viral polymerase, or HBx protein derived from any HBV genotype, e.g., genotype A, B, C, D, E, F, G, and/or H, or combination thereof.

### (1) HBV Core Antigen

As used herein, each of the terms “HBV core antigen,” “HBC” and “core antigen” refers to an HBV antigen capable of inducing an immune response, e.g., a humoral and/or cellular mediated response, against an HBV core protein in a subject. Each of the terms “core,” “core polypeptide,” and “core protein” refers to the HBV viral core protein. Full-length core antigen is typically 183 amino acids in length and includes an assembly domain (amino acids 1 to 149) and a nucleic acid binding domain (amino acids 150 to 183). The 34-residue nucleic acid binding domain is required for pre-genomic RNA encapsidation. This domain also functions as a nuclear import signal. It comprises 17 arginine residues and is highly basic, consistent with its function. HBV core protein is dimeric in solution, with the dimers self-assembling into icosahedral capsids. Each dimer of core protein has four α-helix bundles flanked by an α-helix domain on either side. Truncated HBV core proteins lacking the nucleic acid binding domain are also capable of forming capsids.

In an embodiment of the application, an HBV antigen is a truncated HBV core antigen. As used herein, a “truncated HBV core antigen,” refers to an HBV antigen that does not contain the entire length of an HBV core protein, but is capable of inducing an immune response against the HBV core protein in a subject. For example, an HBV core antigen can be modified to delete one or more amino acids of the highly positively charged (arginine rich) C-terminal nucleic acid binding domain of the core antigen, which typically contains seventeen arginine (R) residues. A truncated HBV core antigen of the application is preferably a C-terminally truncated HBV core protein which does not comprise the HBV core nuclear import signal and/or a truncated HBV core protein from which the C-terminal HBV core nuclear import signal has been deleted. In an embodiment, a truncated HBV core antigen comprises a deletion in the C-terminal nucleic acid binding domain, such as a deletion of 1 to 34 amino acid residues of the C-terminal nucleic acid binding domain, e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, or 34 amino acid residues, preferably a deletion of all 34 amino acid residues. In a preferred embodiment, a truncated HBV core antigen comprises a deletion in the C-terminal nucleic acid binding domain, preferably a deletion of all 34 amino acid residues.

An HBV core antigen of the application can be a consensus sequence derived from multiple HBV genotypes (e.g., genotypes A, B, C, D, E, F, G, and H). As used herein, “consensus sequence” means an artificial sequence of amino acids based on an alignment of amino acid sequences of homologous proteins, e.g., as determined by an alignment (e.g., using Clustal Omega) of amino acid sequences of homologous proteins. It can be the calculated order of most frequent amino acid residues, found at each position in a sequence alignment, based upon sequences of HBV antigens (e.g., core, pol, etc.) from at least 100 natural HBV isolates. A consensus sequence can be non-naturally occurring and different from the native viral sequences. Consensus sequences can be designed by aligning multiple HBV antigen sequences from different sources using a multiple sequence alignment tool, and at variable alignment positions, selecting the most frequent amino acid. Preferably, a consensus sequence of an HBV antigen is derived from HBV genotypes B, C, and D. The term “consensus antigen” is used to refer to an antigen having a consensus sequence.

An exemplary truncated HBV core antigen according to the application lacks the nucleic acid binding function, and is capable of inducing an immune response in a mammal against at least two HBV genotypes. Preferably a truncated HBV core antigen is capable of inducing a T cell response in a mammal against at least HBV genotypes B, C and D. More preferably, a truncated HBV core antigen is capable of inducing a CD8 T cell response in a human subject against at least HBV genotypes A, B, C and D.

Preferably, an HBV core antigen of the application is a consensus antigen, preferably a consensus antigen derived from HBV genotypes B, C, and D, more preferably a truncated consensus antigen derived from HBV genotypes B, C, and D. An exemplary truncated HBV core consensus antigen according to the application consists of an amino acid sequence that is at least 90% identical to SEQ ID NO: 2 or SEQ ID NO: 4, such as at least 90%, 91%, 92%, 93%, 94%, 95%, 95.5%, 96%, 96.5%, 97%, 97.5% 98%, 98.5%, 99%, 99.1%, 99.2%, 99.3%, 99.4%, 99.5%, 99.6%, 99.7%, 99.8%, 99.9%, or 100% identical to SEQ ID NO: 2 or SEQ ID NO: 4. SEQ ID NO: 2 and SEQ ID NO: 4 are core consensus antigens derived from HBV genotypes B, C, and D. SEQ ID NO: 2 and SEQ ID NO: 4 each contain a 34-amino acid C-terminal deletion of the highly positively charged (arginine rich) nucleic acid binding domain of the native core antigen.

In one embodiment of the application, an HBV core antigen is a truncated HBV antigen consisting of the amino acid sequence of SEQ ID NO: 2. In another embodiment, an HBV core antigen is a truncated HBV antigen consisting of the amino acid sequence of SEQ ID NO: 4. In another embodiment, an HBV core antigen further contains a signal sequence operably linked to the N-terminus of a mature HBV core antigen sequence, such as the amino acid sequence of SEQ ID NO: 2 or SEQ ID NO: 4. Preferably, the signal sequence has the amino acid sequence of SEQ ID NO: 9 or SEQ ID NO: 15.

### (2) HBV Polymerase Antigen

As used herein, the term “HBV polymerase antigen,” “HBV Pol antigen” or “HBV pol antigen” refers to an HBV antigen capable of inducing an immune response, e.g., a humoral and/or cellular mediated response, against an HBV polymerase in a subject. Each of the terms “polymerase,” “polymerase polypeptide,” “Pol” and “pol” refers to the HBV viral DNA polymerase. The HBV viral DNA polymerase has four domains, including, from the N terminus to the C terminus, a terminal protein (TP) domain, which acts as a primer for minus-strand DNA synthesis; a spacer that is nonessential for the polymerase functions; a reverse transcriptase (RT) domain for transcription; and a RNase H domain.

In an embodiment of the application, an HBV antigen comprises an HBV Pol antigen, or any immunogenic fragment or combination thereof. An HBV Pol antigen can contain further modifications to improve immunogenicity of the antigen, such as by introducing mutations into the active sites of the polymerase and/or RNase domains to decrease or substantially eliminate certain enzymatic activities.

Preferably, an HBV Pol antigen of the application does not have reverse transcriptase activity and RNase H activity, and is capable of inducing an immune response in a mammal against at least two HBV genotypes. Preferably, an HBV Pol antigen is capable of inducing a T cell response in a mammal against at least HBV genotypes B, C and D. More preferably, an HBV Pol antigen is capable of inducing a CD8 T cell response in a human subject against at least HBV genotypes A, B, C and D.

Thus, in some embodiments, an HBV Pol antigen is an inactivated Pol antigen. In an embodiment, an inactivated HBV Pol antigen comprises one or more amino acid mutations in the active site of the polymerase domain. In another embodiment, an inactivated HBV Pol antigen comprises one or more amino acid mutations in the active site of the RNaseH domain. In a preferred embodiment, an inactivated HBV pol antigen comprises one or more amino acid mutations in the active site of both the polymerase domain and the RNaseH domain. For example, the “YXDD” motif in the polymerase domain of an HBV pol antigen that can be required for nucleotide/metal ion binding can be mutated, e.g., by replacing one or more of the aspartate residues (D) with asparagine residues (N), eliminating or reducing metal coordination function, thereby decreasing or substantially eliminating reverse transcriptase function. Alternatively, or in addition to mutation of the “YXDD” motif, the “DEDD” motif in the RNaseH domain of an HBV pol antigen required for Mg2+ coordination can be mutated, e.g., by replacing one or more aspartate residues (D) with asparagine residues (N) and/or replacing the glutamate residue (E) with glutamine (Q), thereby decreasing or substantially eliminating RNaseH function. In a particular embodiment, an HBV pol antigen is modified by (1) mutating the aspartate residues (D) to asparagine residues (N) in the “YXDD” motif of the polymerase domain; and (2) mutating the first aspartate residue (D) to an asparagine residue (N) and the first glutamate residue (E) to a glutamine residue (N) in the “DEDD” motif of the RNaseH domain, thereby decreasing or substantially eliminating both the reverse transcriptase and RNaseH functions of the pol antigen.

In a preferred embodiment of the application, an HBV pol antigen is a consensus antigen, preferably a consensus antigen derived from HBV genotypes B, C, and D, more preferably an inactivated consensus antigen derived from HBV genotypes B, C, and D. An exemplary HBV pol consensus antigen according to the application comprises an amino acid sequence that is at least 90% identical to SEQ ID NO: 7, such as at least 90%, 91%, 92%, 93%, 94%, 95%, 95.5%, 96%, 96.5%, 97%, 97.5%, 98%, 98.5%, 99%, 99.1%, 99.2%, 99.3%, 99.4%, 99.5%, 99.6%, 99.7%, 99.8%, 99.9% or 100% identical to SEQ ID NO: 7, preferably at least 98% identical to SEQ ID NO: 7, such as at least 98%, 98.5%, 99%, 99.1%, 99.2%, 99.3%, 99.4%, 99.5%, 99.6%, 99.7%, 99.8%, 99.9% or 100% identical to SEQ ID NO: 7. SEQ ID NO: 7 is a pol consensus antigen derived from HBV genotypes B, C, and D comprising four mutations located in the active sites of the polymerase and RNaseH domains. In particular, the four mutations include mutation of the aspartic acid residues (D) to asparagine residues (N) in the “YXDD” motif of the polymerase domain; and mutation of the first aspartate residue (D) to an asparagine residue (N) and mutation of the glutamate residue (E) to a glutamine residue (Q) in the “DEDD” motif of the RNaseH domain.

In a particular embodiment of the application, an HBV pol antigen comprises the amino acid sequence of SEQ ID NO: 7. In other embodiments of the application, an HBV pol antigen consists of the amino acid sequence of SEQ ID NO: 7. In a further embodiment, an HBV pol antigen further contains a signal sequence operably linked to the N-terminus of a mature HBV pol antigen sequence, such as the amino acid sequence of SEQ ID NO: 7. Preferably, the signal sequence has the amino acid sequence of SEQ ID NO: 9 or SEQ ID NO: 15.

### (3) Fusion of HBV Core Antigen and HBV Polymerase Antigen

As used herein the term “fusion protein” or “fusion” refers to a single polypeptide chain having at least two polypeptide domains that are not normally present in a single, natural polypeptide.

In an embodiment of the application, an HBV antigen comprises a fusion protein comprising a truncated HBV core antigen operably linked to an HBV Pol antigen, or an HBV Pol antigen operably linked to a truncated HBV core antigen, preferably via a linker.

For example, in a fusion protein containing a first polypeptide and a second heterologous polypeptide, a linker serves primarily as a spacer between the first and second polypeptides. In an embodiment, a linker is made up of amino acids linked together by peptide bonds, preferably from 1 to 20 amino acids linked by peptide bonds, wherein the amino acids are selected from the 20 naturally occurring amino acids. In an embodiment, the 1 to 20 amino acids are selected from glycine, alanine, proline, asparagine, glutamine, and lysine. Preferably, a linker is made up of a majority of amino acids that are sterically unhindered, such as glycine and alanine. Exemplary linkers are polyglycines, particularly (Gly)5, (Gly)8; poly(Gly-Ala), and polyalanines. One exemplary suitable linker as shown in the Examples below is (AlaGly)n, wherein n is an integer of 2 to 5.

Preferably, a fusion protein of the application is capable of inducing an immune response in a mammal against HBV core and HBV Pol of at least two HBV genotypes. Preferably, a fusion protein is capable of inducing a T cell response in a mammal against at least HBV genotypes B, C and D. More preferably, the fusion protein is capable of inducing a CD8 T cell response in a human subject against at least HBV genotypes A, B, C and D.

In an embodiment of the application, a fusion protein comprises a truncated HBV core antigen having an amino acid sequence at least 90%, such as at least 90%, 91%, 92%, 93%, 94%, 95%, 95.5% 96%, 96.5%, 97%, 97.5% 98%, 98.5%, 99%, 99.1%, 99.2%, 99.3%, 99.4%, 99.5%, 99.6%, 99.7%, 99.8%, 99.9%, or 100% identical to SEQ ID NO: 2 or SEQ ID NO: 4, a linker, and an HBV Pol antigen having an amino acid sequence at least 90%, such as at least 90%, 91%, 92%, 93%, 94%, 95%, 95.5%, 96%, 96.5%, 97%, 97.5%, 98%, 98.5%, 99%, 99.1%, 99.2%, 99.3%, 99.4%, 99.5%, 99.6%, 99.7%, 99.8%, 99.9%, or 100%, identical to SEQ ID NO: 7.

In a preferred embodiment of the application, a fusion protein comprises a truncated HBV core antigen consisting of the amino acid sequence of SEQ ID NO: 2 or SEQ ID NO: 4, a linker comprising (AlaGly)n, wherein n is an integer of 2 to 5, and an HBV Pol antigen having the amino acid sequence of SEQ ID NO: 7. More preferably, a fusion protein according to an embodiment of the application comprises the amino acid sequence of SEQ ID NO: 16.

In one embodiment of the application, a fusion protein further comprises a signal sequence operably linked to the N-terminus of the fusion protein. Preferably, the signal sequence has the amino acid sequence of SEQ ID NO: 9 or SEQ ID NO: 15. In one embodiment, a fusion protein comprises the amino acid sequence of SEQ ID NO: 17.

Additional disclosure on HBV vaccines that can be used for the present invention are described in U.S. patent application Ser. No. 16/223,251, filed Dec. 18, 2018, the contents of the application, more preferably the examples of the application, are hereby incorporated by reference in their entireties.

### Polynucleotides and Vectors

In another general aspect, the application provides a non-naturally occurring nucleic acid molecule encoding an HBV antigen useful for an invention according to embodiments of the application, and vectors comprising the non-naturally occurring nucleic acid. A first or second non-naturally occurring nucleic acid molecule can comprise any polynucleotide sequence encoding an HBV antigen useful for the application, which can be made using methods known in the art in view of the present disclosure. Preferably, a first or second polynucleotide encodes at least one of a truncated HBV core antigen and an HBV polymerase antigen of the application. A polynucleotide can be in the form of RNA or in the form of DNA obtained by recombinant techniques (e.g., cloning) or produced synthetically (e.g., chemical synthesis). The DNA can be single-stranded or double-stranded, or can contain portions of both double-stranded and single-stranded sequence. The DNA can, for example, comprise genomic DNA, cDNA, or combinations thereof. The polynucleotide can also be a DNA/RNA hybrid. The polynucleotides and vectors of the application can be used for recombinant protein production, expression of the protein in host cell, or the production of viral particles. Preferably, a polynucleotide is DNA.

In an embodiment of the application, a first non-naturally occurring nucleic acid molecule comprises a first polynucleotide sequence encoding a truncated HBV core antigen consisting of an amino acid sequence that is at least 90% identical to SEQ ID NO: 2 or SEQ ID NO: 4, such as at least 90%, 91%, 92%, 93%, 94%, 95%, 95.5%, 96%, 96.5%, 97%, 97.5%, 98%, 98.5%, 99%, 99.1%, 99.2%, 99.3%, 99.4%, 99.5%, 99.6%, 99.7%, 99.8%, 99.9% or 100% identical to SEQ ID NO: 2, preferably 98%, 99% or 100% identical to SEQ ID NO: 2 or SEQ ID NO: 4. In a particular embodiment of the application, a first non-naturally occurring nucleic acid molecule comprises a first polynucleotide sequence encoding a truncated HBV core antigen consisting the amino acid sequence of SEQ ID NO: 2 or SEQ ID NO: 4.

Examples of polynucleotide sequences of the application encoding a truncated HBV core antigen consisting of the amino acid sequence of SEQ ID NO: 2 or SEQ ID NO: 4 include, but are not limited to, a polynucleotide sequence at least 90% identical to SEQ ID NO: 1 or SEQ ID NO: 3, such as at least 90%, 91%, 92%, 93%, 94%, 95%, 95.5%, 96%, 96.5%, 97%, 97.5%, 98%, 98.5%, 99%, 99.1%, 99.2%, 99.3%, 99.4%, 99.5%, 99.6%, 99.7%, 99.8%, 99.9% or 100% identical to SEQ ID NO: 1 or SEQ ID NO: 3, preferably 98%, 99% or 100% identical to SEQ ID NO: 1 or SEQ ID NO: 3. Exemplary non-naturally occurring nucleic acid molecules encoding a truncated HBV core antigen have the polynucleotide sequence of SEQ ID NOs: 1 or 3.

In another embodiment, a first non-naturally occurring nucleic acid molecule further comprises a coding sequence for a signal sequence that is operably linked to the N-terminus of the HBV core antigen sequence. Preferably, the signal sequence has the amino acid sequence of SEQ ID NO: 9 or SEQ ID NO: 15. More preferably, the coding sequence for a signal sequence comprises the polynucleotide sequence of SEQ ID NO: 8 or SEQ ID NO: 14.

In an embodiment of the application, a second non-naturally occurring nucleic acid molecule comprises a second polynucleotide sequence encoding an HBV polymerase antigen comprising an amino acid sequence that is at least 90% identical to SEQ ID NO: 7, such as at least 90%, 91%, 92%, 93%, 94%, 95%, 95.5%, 96%, 96.5%, 97%, 97.5%, 98%, 98.5%, 99%, 99.1%, 99.2%, 99.3%, 99.4%, 99.5%, 99.6%, 99.7%, 99.8%, 99.9% or 100% identical to SEQ ID NO: 7, preferably 100% identical to SEQ ID NO: 7. In a particular embodiment of the application, a second non-naturally occurring nucleic acid molecule comprises a second polynucleotide sequence encoding an HBV polymerase antigen consisting of the amino acid sequence of SEQ ID NO: 7.

Examples of polynucleotide sequences of the application encoding an HBV Pol antigen comprising the amino acid sequence of at least 90% identical to SEQ ID NO: 7 include, but are not limited to, a polynucleotide sequence at least 90% identical to SEQ ID NO: 5 or SEQ ID NO: 6, such as at least 90%, 91%, 92%, 93%, 94%, 95%, 95.5%, 96%, 96.5%, 97%, 97.5%, 98%, 98.5%, 99%, 99.1%, 99.2%, 99.3%, 99.4%, 99.5%, 99.6%, 99.7%, 99.8%, 99.9% or 100% identical to SEQ ID NO: 5 or SEQ ID NO: 6, preferably 98%, 99% or 100% identical to SEQ ID NO: 5 or SEQ ID NO: 6. Exemplary non-naturally occurring nucleic acid molecules encoding an HBV pol antigen have the polynucleotide sequence of SEQ ID NOs: 5 or 6.

In another embodiment, a second non-naturally occurring nucleic acid molecule further comprises a coding sequence for a signal sequence that is operably linked to the N-terminus of the HBV pol antigen sequence, such as the amino acid sequence of SEQ ID NO: 7. Preferably, the signal sequence has the amino acid sequence of SEQ ID NO: 9 or SEQ ID NO: 15. More preferably, the coding sequence for a signal sequence comprises the polynucleotide sequence of SEQ ID NO: 8 or SEQ ID NO: 14.

In another embodiment of the application, a non-naturally occurring nucleic acid molecule encodes an HBV antigen fusion protein comprising a truncated HBV core antigen operably linked to an HBV Pol antigen, or an HBV Pol antigen operably linked to a truncated HBV core antigen. In a particular embodiment, a non-naturally occurring nucleic acid molecule of the application encodes a truncated HBV core antigen consisting of an amino acid sequence that is at least 90% identical to SEQ ID NO: 2 or SEQ ID NO: 4, such as at least 90%, 91%, 92%, 93%, 94%, 95%, 95.5%, 96%, 96.5%, 97%, 97.5%, 98%, 98.5%, 99%, 99.1%, 99.2%, 99.3%, 99.4%, 99.5%, 99.6%, 99.7%, 99.8%, 99.9% or 100% identical to SEQ ID NO: 2 or SEQ ID NO: 4, preferably 100% identical to SEQ ID NO: 2 or SEQ ID NO: 4, more preferably 100% identical to SEQ ID NO: 2 or SEQ ID NO:4; a linker; and an HBV polymerase antigen comprising an amino acid sequence that is at least 90% identical to SEQ ID NO: 7, such as at least 90%, 91%, 92%, 93%, 94%, 95%, 95.5%, 96%, 96.5%, 97%, 97.5%, 98%, 98.5%, 99%, 99.1%, 99.2%, 99.3%, 99.4%, 99.5%, 99.6%, 99.7%, 99.8%, 99.9% or 100% identical to SEQ ID NO: 7, preferably 98%, 99% or 100% identical to SEQ ID NO: 7. In a particular embodiment of the application, a non-naturally occurring nucleic acid molecule encodes a fusion protein comprising a truncated HBV core antigen consisting of the amino acid sequence of SEQ ID NO: 2 or SEQ ID NO: 4, a linker comprising (AlaGly)n, wherein n is an integer of 2 to 5; and an HBV Pol antigen comprising the amino acid sequence of SEQ ID NO: 7. In a particular embodiment of the application, a non-naturally occurring nucleic acid molecule encodes an HBV antigen fusion protein comprising the amino acid sequence of SEQ ID NO: 16.

Examples of polynucleotide sequences of the application encoding an HBV antigen fusion protein include, but are not limited to, a polynucleotide sequence at least 90% identical to SEQ ID NO: 1 or SEQ ID NO: 3, such as at least 90%, 91%, 92%, 93%, 94%, 95%, 95.5%, 96%, 96.5%, 97%, 97.5%, 98%, 98.5%, 99%, 99.1%, 99.2%, 99.3% 99.4%, 99.5%, 99.6%, 99.7%, 99.8%, 99.9% or 100% identical to SEQ ID NO: 1 or SEQ ID NO: 3, preferably 98%, 99% or 100% identical to SEQ ID NO: 1 or SEQ ID NO: 3, operably linked to a linker coding sequence at least 90% identical to SEQ ID NO: 11, such as at least 90%, 91%, 92%, 93%, 94%, 95%, 95.5%, 96%, 96.5%, 97%, 97.5% 98%, 98.5%, 99%, 99.1%, 99.2%, 99.3%, 99.4%, 99.5%, 99.6%, 99.7%, 99.8%, 99.9% or 100% identical to SEQ ID NO: 11, preferably 98%, 99% or 100% identical to SEQ ID NO: 11, which is further operably linked a polynucleotide sequence at least 90% identical to SEQ ID NO: 5 or SEQ ID NO: 6, such as at least 90%, 91%, 92%, 93%, 94%, 95%, 95.5%, 96%, 96.5%, 97%, 97.5%, 98%, 98.5%, 99%, 99.1%, 99.2%, 99.3%, 99.4%, 99.5%, 99.6%, 99.7%, 99.8%, 99.9% or 100% identical to SEQ ID NO: 5 or SEQ ID NO: 6, preferably 98%, 99% or 100% identical to SEQ ID NO: 5 or SEQ ID NO: 6. In particular embodiments of the application, a non-naturally occurring nucleic acid molecule encoding an HBV antigen fusion protein comprises SEQ ID NO: 1 or SEQ ID NO: 3, operably linked to SEQ ID NO: 11, which is further operably linked to SEQ ID NO: 5 or SEQ ID NO: 6.

In another embodiment, a non-naturally occurring nucleic acid molecule encoding an HBV fusion further comprises a coding sequence for a signal sequence that is operably linked to the N-terminus of the HBV fusion sequence, such as the amino acid sequence of SEQ ID NO: 16. Preferably, the signal sequence has the amino acid sequence of SEQ ID NO: 9 or SEQ ID NO: 15. More preferably, the coding sequence for a signal sequence comprises the polynucleotide sequence of SEQ ID NO: 8 or SEQ ID NO: 14. In one embodiment, the encoded fusion protein with the signal sequence comprises the amino acid sequence of SEQ ID NO: 17.

The application also relates to a vector comprising the first and/or second non-naturally occurring nucleic acid molecules. As used herein, a “vector” is a nucleic acid molecule used to carry genetic material into another cell, where it can be replicated and/or expressed. Any vector known to those skilled in the art in view of the present disclosure can be used. Examples of vectors include, but are not limited to, plasmids, viral vectors (bacteriophage, animal viruses, and plant viruses), cosmids, and artificial chromosomes (e.g., YACs). Preferably, a vector is a DNA plasmid. A vector can be a DNA vector or an RNA vector. One of ordinary skill in the art can construct a vector of the application through standard recombinant techniques in view of the present disclosure.

A vector of the application can be an expression vector. As used herein, the term “expression vector” refers to any type of genetic construct comprising a nucleic acid coding for an RNA capable of being transcribed. Expression vectors include, but are not limited to, vectors for recombinant protein expression, such as a DNA plasmid or a viral vector, and vectors for delivery of nucleic acid into a subject for expression in a tissue of the subject, such as a DNA plasmid or a viral vector. It will be appreciated by those skilled in the art that the design of the expression vector can depend on such factors as the choice of the host cell to be transformed, the level of expression of protein desired, etc.

Vectors of the application can contain a variety of regulatory sequences. As used herein, the term “regulatory sequence” refers to any sequence that allows, contributes or modulates the functional regulation of the nucleic acid molecule, including replication, duplication, transcription, splicing, translation, stability and/or transport of the nucleic acid or one of its derivative (i.e. mRNA) into the host cell or organism. In the context of the disclosure, this term encompasses promoters, enhancers and other expression control elements (e.g., polyadenylation signals and elements that affect mRNA stability).

In some embodiments of the application, a vector is a non-viral vector. Examples of non-viral vectors include, but are not limited to, DNA plasmids, bacterial artificial chromosomes, yeast artificial chromosomes, bacteriophages, etc. Examples of non-viral vectors include, but are not limited to, RNA replicon, mRNA replicon, modified mRNA replicon or self-amplifying mRNA, closed linear deoxyribonucleic acid, e.g. a linear covalently closed DNA such as linear covalently closed double stranded DNA molecule. Preferably, a non-viral vector is a DNA plasmid. A “DNA plasmid”, which is used interchangeably with “DNA plasmid vector,” “plasmid DNA” or “plasmid DNA vector,” refers to a double-stranded and generally circular DNA sequence that is capable of autonomous replication in a suitable host cell. DNA plasmids used for expression of an encoded polynucleotide typically comprise an origin of replication, a multiple cloning site, and a selectable marker, which for example, can be an antibiotic resistance gene. Examples of DNA plasmids suitable that can be used include, but are not limited to, commercially available expression vectors for use in well-known expression systems (including both prokaryotic and eukaryotic systems), such as pSE420 (Invitrogen, San Diego, Calif.), which can be used for production and/or expression of protein in Escherichia coli; pYES2 (Invitrogen, Thermo Fisher Scientific), which can be used for production and/or expression in Saccharomyces cerevisiae strains of yeast; MAXBAC® complete baculovirus expression system (Thermo Fisher Scientific), which can be used for production and/or expression in insect cells; pcDNA™ or pcDNA3™ (Life Technologies, Thermo Fisher Scientific), which can be used for high level constitutive protein expression in mammalian cells; and pVAX or pVAX-1 (Life Technologies, Thermo Fisher Scientific), which can be used for high-level transient expression of a protein of interest in most mammalian cells. The backbone of any commercially available DNA plasmid can be modified to optimize protein expression in the host cell, such as to reverse the orientation of certain elements (e.g., origin of replication and/or antibiotic resistance cassette), replace a promoter endogenous to the plasmid (e.g., the promoter in the antibiotic resistance cassette), and/or replace the polynucleotide sequence encoding transcribed proteins (e.g., the coding sequence of the antibiotic resistance gene), by using routine techniques and readily available starting materials. (See e.g., Sambrook et al., Molecular Cloning a Laboratory Manual, Second Ed. Cold Spring Harbor Press (1989)).

Preferably, a DNA plasmid is an expression vector suitable for protein expression in mammalian host cells. Expression vectors suitable for protein expression in mammalian host cells include, but are not limited to, pcDNA™, pcDNA3™, pVAX, pVAX-1, ADVAX, NTC8454, etc. Preferably, an expression vector is based on pVAX-1, which can be further modified to optimize protein expression in mammalian cells. pVAX-1 is commonly used plasmid in DNA vaccines, and contains a strong human intermediate early cytomegalovirus (CMV-IE) promoter followed by the bovine growth hormone (bGH)-derived polyadenylation sequence (pA). pVAX-1 further contains a pUC origin of replication and kanamycin resistance gene driven by a small prokaryotic promoter that allows for bacterial plasmid propagation.

A vector of the application can also be a viral vector. In general, viral vectors are genetically engineered viruses carrying modified viral DNA or RNA that has been rendered non-infectious, but still contains viral promoters and transgenes, thus allowing for translation of the transgene through a viral promoter. Because viral vectors are frequently lacking infectious sequences, they require helper viruses or packaging lines for large-scale transfection. Examples of viral vectors that can be used include, but are not limited to, adenoviral vectors, adeno-associated virus vectors, pox virus vectors, enteric virus vectors, Venezuelan Equine Encephalitis virus vectors, Semliki Forest Virus vectors, Tobacco Mosaic Virus vectors, lentiviral vectors, etc. Examples of viral vectors that can be used include, but are not limited to, arenavirus viral vectors, replication-deficient arenavirus viral vectors or replication-competent arenavirus viral vectors, bi-segmented or tri-segmented arenavirus, infectious arenavirus viral vectors, nucleic acids which comprise an arenavirus genomic segment wherein one open reading frame of the genomic segment is deleted or functionally inactivated (and replaced by a nucleic acid encoding an HBV antigen as described herein), arenavirus such as lymphocytic choriomeningitidis virus (LCMV), e.g., clone 13 strain or MP strain, and arenavirus such as Junin virus e.g., Candid #1 strain. The vector can also be a non-viral vector.

Preferably, a viral vector is an adenovirus vector, e.g., a recombinant adenovirus vector. A recombinant adenovirus vector can for instance be derived from a human adenovirus (HAdV, or AdHu), or a simian adenovirus such as chimpanzee or gorilla adenovirus (ChAd, AdCh, or SAdV) or rhesus adenovirus (rhAd). Preferably, an adenovirus vector is a recombinant human adenovirus vector, for instance a recombinant human adenovirus serotype 26, or any one of recombinant human adenovirus serotype 5, 4, 35, 7, 48, etc. In other embodiments, an adenovirus vector is a rhAd vector, e.g. rhAd51, rhAd52 or rhAd53.

The vector can also be a linear covalently closed double-stranded DNA vector. As used herein, a “linear covalently closed double-stranded DNA vector” refers to a closed linear deoxyribonucleic acid (DNA) that is structurally distinct from a plasmid DNA. It has many of the advantages of plasmid DNA as well as a minimal cassette size similar to RNA strategies. For example, it can be a vector cassette generally comprising an encoded antigenic sequence, a promoter, a polyadenylation sequence, and telomeric ends. The plasmid-free construct can be synthesized through an enzymatic process without the need for bacterial sequences. Examples of suitable linear covalently closed DNA vectors include, but are not limited to, commercially available expression vectors such as ‘Doggybone™ closed linear DNA’ (dbDNA™) (Touchlight Genetics Ltd.; London, England). See, e.g., Scott et al, Hum Vaccin Immunother. 2015 August; 11(8): 1972-1982, the entire content of which is incorporated herein by reference. Some examples of linear covalently closed double-stranded DNA vectors, compositions and methods to create and use such vectors for delivering DNA molecules, such as active molecules of this invention, are described in US2012/0282283, US2013/0216562, and US2018/0037943, the relevant content of each of which is hereby incorporated by reference in its entirety.

A recombinant vector useful for the application can be prepared using methods known in the art in view of the present disclosure. For example, in view of the degeneracy of the genetic code, several nucleic acid sequences can be designed that encode the same polypeptide. A polynucleotide encoding an HBV antigen of the application can optionally be codon-optimized to ensure proper expression in the host cell (e.g., bacterial or mammalian cells). Codon-optimization is a technology widely applied in the art, and methods for obtaining codon-optimized polynucleotides will be well known to those skilled in the art in view of the present disclosure.

A vector of the application, e.g., a DNA plasmid, a viral vector (particularly an adenoviral vector), an RNA vector (such as a self-replicating RNA replicon), or a linear covalently closed double-stranded DNA vector, can comprise any regulatory elements to establish conventional function(s) of the vector, including but not limited to replication and expression of the HBV antigen(s) encoded by the polynucleotide sequence of the vector. Regulatory elements include, but are not limited to, a promoter, an enhancer, a polyadenylation signal, translation stop codon, a ribosome binding element, a transcription terminator, selection markers, origin of replication, etc. A vector can comprise one or more expression cassettes. An “expression cassette” is part of a vector that directs the cellular machinery to make RNA and protein. An expression cassette typically comprises three components: a promoter sequence, an open reading frame, and a 3′-untranslated region (UTR) optionally comprising a polyadenylation signal. An open reading frame (ORF) is a reading frame that contains a coding sequence of a protein of interest (e.g., HBV antigen) from a start codon to a stop codon. Regulatory elements of the expression cassette can be operably linked to a polynucleotide sequence encoding an HBV antigen of interest. As used herein, the term “operably linked” is to be taken in its broadest reasonable context, and refers to a linkage of polynucleotide elements in a functional relationship. A polynucleotide is “operably linked” when it is placed into a functional relationship with another polynucleotide. For instance, a promoter is operably linked to a coding sequence if it affects the transcription of the coding sequence. Any components suitable for use in an expression cassette described herein can be used in any combination and in any order to prepare vectors of the application.

A vector can comprise a promoter sequence, preferably within an expression cassette, to control expression of an HBV antigen of interest. The term “promoter” is used in its conventional sense, and refers to a nucleotide sequence that initiates the transcription of an operably linked nucleotide sequence. A promoter is located on the same strand near the nucleotide sequence it transcribes. Promoters can be a constitutive, inducible, or repressible. Promoters can be naturally occurring or synthetic. A promoter can be derived from sources including viral, bacterial, fungal, plants, insects, and animals. A promoter can be a homologous promoter (i.e., derived from the same genetic source as the vector) or a heterologous promoter (i.e., derived from a different vector or genetic source). For example, if the vector to be employed is a DNA plasmid, the promoter can be endogenous to the plasmid (homologous) or derived from other sources (heterologous). Preferably, the promoter is located upstream of the polynucleotide encoding an HBV antigen within an expression cassette.

Examples of promoters that can be used include, but are not limited to, a promoter from simian virus 40 (SV40), a mouse mammary tumor virus (MMTV) promoter, a human immunodeficiency virus (HIV) promoter such as the bovine immunodeficiency virus (BIV) long terminal repeat (LTR) promoter, a Moloney virus promoter, an avian leukosis virus (ALV) promoter, a cytomegalovirus (CMV) promoter such as the CMV immediate early promoter (CMV-IE), Epstein Barr virus (EBV) promoter, or a Rous sarcoma virus (RSV) promoter. A promoter can also be a promoter from a human gene such as human actin, human myosin, human hemoglobin, human muscle creatine, or human metalothionein. A promoter can also be a tissue specific promoter, such as a muscle or skin specific promoter, natural or synthetic.

Preferably, a promoter is a strong eukaryotic promoter, preferably a cytomegalovirus immediate early (CMV-IE) promoter. A nucleotide sequence of an exemplary CMV-IE promoter is shown in SEQ ID NO: 18 or SEQ ID NO: 19.

A vector can comprise additional polynucleotide sequences that stabilize the expressed transcript, enhance nuclear export of the RNA transcript, and/or improve transcriptional-translational coupling. Examples of such sequences include polyadenylation signals and enhancer sequences. A polyadenylation signal is typically located downstream of the coding sequence for a protein of interest (e.g., an HBV antigen) within an expression cassette of the vector. Enhancer sequences are regulatory DNA sequences that, when bound by transcription factors, enhance the transcription of an associated gene. An enhancer sequence is preferably located upstream of the polynucleotide sequence encoding an HBV antigen, but downstream of a promoter sequence within an expression cassette of the vector.

Any polyadenylation signal known to those skilled in the art in view of the present disclosure can be used. For example, the polyadenylation signal can be a SV40 polyadenylation signal, LTR polyadenylation signal, bovine growth hormone (bGH) polyadenylation signal, human growth hormone (hGH) polyadenylation signal, or human β-globin polyadenylation signal. Preferably, a polyadenylation signal is a bovine growth hormone (bGH) polyadenylation signal or a SV40 polyadenylation signal. A nucleotide sequence of an exemplary bGH polyadenylation signal is shown in SEQ ID NO: 20. A nucleotide sequence of an exemplary SV40 polyadenylation signal is shown in SEQ ID NO: 13.

Any enhancer sequence known to those skilled in the art in view of the present disclosure can be used. For example, an enhancer sequence can be human actin, human myosin, human hemoglobin, human muscle creatine, or a viral enhancer, such as one from CMV, HA, RSV, or EBV. Examples of particular enhancers include, but are not limited to, Woodchuck HBV Post-transcriptional regulatory element (WPRE), intron/exon sequence derived from human apolipoprotein A1 precursor (ApoAI), untranslated R-U5 domain of the human T-cell leukemia virus type 1 (HTLV-1) long terminal repeat (LTR), a splicing enhancer, a synthetic rabbit β-globin intron, or any combination thereof. Preferably, an enhancer sequence is a composite sequence of three consecutive elements of the untranslated R-U5 domain of HTLV-1 LTR, rabbit β-globin intron, and a splicing enhancer, which is referred to herein as “a triple enhancer sequence.” A nucleotide sequence of an exemplary triple enhancer sequence is shown in SEQ ID NO: 10. Another exemplary enhancer sequence is an ApoAI gene fragment shown in SEQ ID NO: 12.

A vector can comprise a polynucleotide sequence encoding a signal peptide sequence. Preferably, the polynucleotide sequence encoding the signal peptide sequence is located upstream of the polynucleotide sequence encoding an HBV antigen. Signal peptides typically direct localization of a protein, facilitate secretion of the protein from the cell in which it is produced, and/or improve antigen expression and cross-presentation to antigen-presenting cells. A signal peptide can be present at the N-terminus of an HBV antigen when expressed from the vector, but is cleaved off by signal peptidase, e.g., upon secretion from the cell. An expressed protein in which a signal peptide has been cleaved is often referred to as the “mature protein.” Any signal peptide known in the art in view of the present disclosure can be used. For example, a signal peptide can be a cystatin S signal peptide; an immunoglobulin (Ig) secretion signal, such as the Ig heavy chain gamma signal peptide SPIgG or the Ig heavy chain epsilon signal peptide SPIgE.

Preferably, a signal peptide sequence is a cystatin S signal peptide. Exemplary nucleic acid and amino acid sequences of a cystatin S signal peptide are shown in SEQ ID NOs: 8 and 9, respectively. Exemplary nucleic acid and amino acid sequences of an immunoglobulin secretion signal are shown in SEQ ID NOs: 14 and 15, respectively.

A vector, such as a DNA plasmid, can also include a bacterial origin of replication and an antibiotic resistance expression cassette for selection and maintenance of the plasmid in bacterial cells, e.g., E. coli. Bacterial origins of replication and antibiotic resistance cassettes can be located in a vector in the same orientation as the expression cassette encoding an HBV antigen, or in the opposite (reverse) orientation. An origin of replication (ORI) is a sequence at which replication is initiated, enabling a plasmid to reproduce and survive within cells. Examples of ORIs suitable for use in the application include, but are not limited to ColE1, pMB1, pUC, pSC101, R6K, and 15A, preferably pUC. An exemplary nucleotide sequence of a pUC ORI is shown in SEQ ID NO: 21.

Expression cassettes for selection and maintenance in bacterial cells typically include a promoter sequence operably linked to an antibiotic resistance gene. Preferably, the promoter sequence operably linked to an antibiotic resistance gene differs from the promoter sequence operably linked to a polynucleotide sequence encoding a protein of interest, e.g., HBV antigen. The antibiotic resistance gene can be codon optimized, and the sequence composition of the antibiotic resistance gene is normally adjusted to bacterial, e.g., E. coli, codon usage. Any antibiotic resistance gene known to those skilled in the art in view of the present disclosure can be used, including, but not limited to, kanamycin resistance gene (Kanr), ampicillin resistance gene (Ampr), and tetracycline resistance gene (Tetr), as well as genes conferring resistance to chloramphenicol, bleomycin, spectinomycin, carbenicillin, etc.

Preferably, an antibiotic resistance gene in the antibiotic expression cassette of a vector is a kanamycin resistance gene (Kanr). The sequence of Kanr gene is shown in SEQ ID NO: 22. Preferably, the Kanr gene is codon optimized. An exemplary nucleic acid sequence of a codon optimized Kanr gene is shown in SEQ ID NO: 23. The Kanr can be operably linked to its native promoter, or the Kanr gene can be linked to a heterologous promoter. In a particular embodiment, the Kanr gene is operably linked to the ampicillin resistance gene (Ampr) promoter, known as the bla promoter. An exemplary nucleotide sequence of a bla promoter is shown in SEQ ID NO: 24.

In a particular embodiment of the application, a vector is a DNA plasmid comprising an expression cassette including a polynucleotide encoding at least one of an HBV antigen selected from the group consisting of an HBV pol antigen comprising an amino acid sequence at least 90%, such as 90%, 91%, 92%, 93%, 94%, 95%, 96, 97%, preferably at least 98%, such as at least 98%, 98.5%, 99%, 99.1%, 99.2%, 99.3%, 99.4%, 99.5%, 99.6%, 99.7%, 99.8%, 99.9% or 100%, identical to SEQ ID NO: 7, and a truncated HBV core antigen consisting of the amino acid sequence at least 95%, such as 95%, 96, 97%, preferably at least 98%, such as at least 98%, 98.5%, 99%, 99.1%, 99.2%, 99.3%, 99.4%, 99.5%, 99.6%, 99.7%, 99.8%, 99.9% or 100%, identical of SEQ ID NO: 2 or SEQ ID NO: 4; an upstream sequence operably linked to the polynucleotide encoding the HBV antigen comprising, from 5′ end to 3′ end, a promoter sequence, preferably a CMV promoter sequence of SEQ ID NO: 18, an enhancer sequence, preferably a triple enhancer sequence of SEQ ID NO: 10, and a polynucleotide sequence encoding a signal peptide sequence, preferably a cystatin S signal peptide having the amino acid sequence of SEQ ID NO: 9; and a downstream sequence operably linked to the polynucleotide encoding the HBV antigen comprising a polyadenylation signal, preferably a bGH polyadenylation signal of SEQ ID NO: 20. Such vector further comprises an antibiotic resistance expression cassette including a polynucleotide encoding an antibiotic resistance gene, preferably a Kan^{r} gene, more preferably a codon optimized Kan^{r} gene of at least 90% identical to SEQ ID NO: 23, such as at least 90%, 91%, 92%, 93%, 94%, 95%, 95.5%, 96%, 96.5%, 97%, 97.5% 98%, 98.5%, 99%, 99.1%, 99.2%, 99.3% 99.4%, 99.5%, 99.6%, 99.7%, 99.8%, 99.9% or 100% identical to SEQ ID NO: 23, preferably 100% identical to SEQ ID NO: 23, operably linked to an Ampr (bla) promoter of SEQ ID NO: 24, upstream of and operably linked to the polynucleotide encoding the antibiotic resistance gene; and an origin of replication, preferably a pUC on of SEQ ID NO: 21. Preferably, the antibiotic resistance cassette and the origin of replication are present in the plasmid in the reverse orientation relative to the HBV antigen expression cassette.

In another particular embodiment of the application, a vector is a viral vector, preferably an adenoviral vector, more preferably an Ad26 or Ad35 vector, comprising an expression cassette including a polynucleotide encoding at least one of an HBV antigen selected from the group consisting of an HBV pol antigen comprising an amino acid sequence at least 90%, such as 90%, 91%, 92%, 93%, 94%, 95%, 96, 97%, preferably at least 98%, such as at least 98%, 98.5%, 99%, 99.1%, 99.2%, 99.3%, 99.4%, 99.5%, 99.6%, 99.7%, 99.8%, 99.9% or 100%, identical to SEQ ID NO: 7, and a truncated HBV core antigen consisting of the amino acid sequence at least 95%, such as 95%, 96, 97%, preferably at least 98%, such as at least 98%, 98.5%, 99%, 99.1%, 99.2%, 99.3%, 99.4%, 99.5%, 99.6%, 99.7%, 99.8%, 99.9% or 100%, identical of SEQ ID NO: 2 or SEQ ID NO: 4; an upstream sequence operably linked to the polynucleotide encoding the HBV antigen comprising, from 5′ end to 3′ end, a promoter sequence, preferably a CMV promoter sequence of SEQ ID NO: 19, an enhancer sequence, preferably an ApoAI gene fragment sequence of SEQ ID NO: 12, and a polynucleotide sequence encoding a signal peptide sequence, preferably an immunoglobulin secretion signal having the amino acid sequence of SEQ ID NO: 15; and a downstream sequence operably linked to the polynucleotide encoding the HBV antigen comprising a polyadenylation signal, preferably a SV40 polyadenylation signal of SEQ ID NO: 13.

In an embodiment of the application, a vector, such as a plasmid DNA vector or a viral vector (preferably an adenoviral vector, more preferably an Ad26 or Ad35 vector), encodes an HBV Pol antigen having the amino acid sequence of SEQ ID NO: 7. Preferably, the vector comprises a coding sequence for the HBV Pol antigen that is at least 90% identical to the polynucleotide sequence of SEQ ID NO: 5 or 6, such as 90%, 91%, 92%, 93%, 94%, 95%, 95.5%, 96%, 96.5%, 97%, 97.5%, 98%, 98.5%, 99%, 99.1%, 99.2%, 99.3%, 99.4%, 99.5%, 99.6%, 99.7%, 99.8%, 99.9% or 100% identical to SEQ ID NO: 5 or 6, preferably 100% identical to SEQ ID NO: 5 or 6.

In an embodiment of the application, a vector, such as a plasmid DNA vector or a viral vector (preferably an adenoviral vector, more preferably an Ad26 or Ad35 vector), encodes a truncated HBV core antigen consisting of the amino acid sequence of SEQ ID NO: 2 or SEQ ID NO: 4. Preferably, the vector comprises a coding sequence for the truncated HBV core antigen that is at least 90% identical to the polynucleotide sequence of SEQ ID NO: 1 or SEQ ID NO: 3, such as 90%, 91%, 92%, 93%, 94%, 95%, 95.5%, 96%, 96.5%, 97%, 97.5%, 98%, 98.5%, 99%, 99.1%, 99.2%, 99.3%, 99.4%, 99.5%, 99.6%, 99.7%, 99.8%, 99.9% or 100% identical to SEQ ID NO: 1 or SEQ ID NO: 3, preferably 100% identical to SEQ ID NO: 1 or SEQ ID NO: 3.

In yet another embodiment of the application, a vector, such as a plasmid DNA vector or a viral vector (preferably an adenoviral vector, more preferably an Ad26 or Ad35 vector), encodes a fusion protein comprising an HBV Pol antigen having the amino acid sequence of SEQ ID NO: 7 and a truncated HBV core antigen consisting of the amino acid sequence of SEQ ID NO: 1 or SEQ ID NO: 3. Preferably, the vector comprises a coding sequence for the fusion, which contains a coding sequence for the truncated HBV core antigen at least 90% identical to SEQ ID NO: 1 or SEQ ID NO: 3, such as at least 90%, 91%, 92%, 93%, 94%, 95%, 95.5%, 96%, 96.5%, 97%, 97.5% 98%, 98.5%, 99%, 99.1%, 99.2%, 99.3%, 99.4%, 99.5%, 99.6%, 99.7%, 99.8%, 99.9% or 100% identical to SEQ ID NO: 1 or SEQ ID NO: 3, preferably 98%, 99% or 100% identical to SEQ ID NO: 1 or SEQ ID NO: 3, more preferably SEQ ID NO: 1 or SEQ ID NO: 3, operably linked to a coding sequence for the HBV Pol antigen at least 90% identical to SEQ ID NO: 5 or SEQ ID NO: 6, such as at least 90%, 91%, 92%, 93%, 94%, 95%, 95.5%, 96%, 96.5%, 97%, 97.5%, 98%, 98.5%, 99%, 99.1%, 99.2%, 99.3% 99.4%, 99.5%, 99.6%, 99.7%, 99.8%, 99.9% or 100% identical to SEQ ID NO: 5 or SEQ ID NO: 6, preferably 98%, 99% or 100% identical to SEQ ID NO: 5 or SEQ ID NO: 6, more preferably SEQ ID NO: 5 or SEQ ID NO: 6. Preferably, the coding sequence for the truncated HBV core antigen is operably linked to the coding sequence for the HBV Pol antigen via a coding sequence for a linker at least 90% identical to SEQ ID NO: 11, such as at least 90%, 91%, 92%, 93%, 94%, 95%, 95.5%, 96%, 96.5%, 97%, 97.5% 98%, 98.5%, 99%, 99.1%, 99.2%, 99.3%, 99.4%, 99.5%, 99.6%, 99.7%, 99.8%, 99.9% or 100% identical to SEQ ID NO: 11, preferably 98%, 99% or 100% identical to SEQ ID NO: 11. In particular embodiments of the application, a vector comprises a coding sequence for the fusion having SEQ ID NO: 1 or SEQ ID NO: 3 operably linked to SEQ ID NO: 11, which is further operably linked to SEQ ID NO: 5 or SEQ ID NO: 6.

The polynucleotides and expression vectors encoding the HBV antigens of the application can be made by any method known in the art in view of the present disclosure. For example, a polynucleotide encoding an HBV antigen can be introduced or “cloned” into an expression vector using standard molecular biology techniques, e.g., polymerase chain reaction (PCR), etc., which are well known to those skilled in the art.

### Cells, Polypeptides and Antibodies

The application also provides cells, preferably isolated cells, comprising any of the polynucleotides and vectors described herein. The cells can, for instance, be used for recombinant protein production, or for the production of viral particles.

Embodiments of the application thus also relate to a method of making an HBV antigen of the application. The method comprises transfecting a host cell with an expression vector comprising a polynucleotide encoding an HBV antigen of the application operably linked to a promoter, growing the transfected cell under conditions suitable for expression of the HBV antigen, and optionally purifying or isolating the HBV antigen expressed in the cell. The HBV antigen can be isolated or collected from the cell by any method known in the art including affinity chromatography, size exclusion chromatography, etc. Techniques used for recombinant protein expression will be well known to one of ordinary skill in the art in view of the present disclosure. The expressed HBV antigens can also be studied without purifying or isolating the expressed protein, e.g., by analyzing the supernatant of cells transfected with an expression vector encoding the HBV antigen and grown under conditions suitable for expression of the HBV antigen.

Thus, also provided are non-naturally occurring or recombinant polypeptides comprising an amino acid sequence that is at least 90% identical to the amino acid sequence of SEQ ID NO: 2, SEQ ID NO: 4, or SEQ ID NO: 7. As described above and below, isolated nucleic acid molecules encoding these sequences, vectors comprising these sequences operably linked to a promoter, and compositions comprising the polypeptide, polynucleotide, or vector are also contemplated by the application.

In an embodiment of the application, a recombinant polypeptide comprises an amino acid sequence that is at least 90% identical to the amino acid sequence of SEQ ID NO: 2, such as 90%, 91%, 92%, 93%, 94%, 95%, 95.5%, 96%, 96.5%, 97%, 97.5%, 98%, 98.5%, 99%, 99.1%, 99.2%, 99.3%, 99.4%, 99.5%, 99.6%, 99.7%, 99.8%, 99.9% or 100% identical to SEQ ID NO: 2. Preferably, a non-naturally occurring or recombinant polypeptide consists of SEQ ID NO: 2.

In another embodiment of the application, a non-naturally occurring or recombinant polypeptide comprises an amino acid sequence that is at least 90% identical to the amino acid sequence of SEQ ID NO: 4, such as 90%, 91%, 92%, 93%, 94%, 95%, 95.5%, 96%, 96.5%, 97%, 97.5%, 98%, 98.5%, 99%, 99.1%, 99.2%, 99.3%, 99.4%, 99.5%, 99.6%, 99.7%, 99.8%, 99.9% or 100% identical to SEQ ID NO: 4. Preferably, a non-naturally occurring or recombinant polypeptide comprises SEQ ID NO: 4.

In another embodiment of the application, a non-naturally occurring or recombinant polypeptide comprises an amino acid sequence that is at least 90% identical to the amino acid sequence of SEQ ID NO: 7, such as 90%, 91%, 92%, 93%, 94%, 95%, 95.5%, 96%, 96.5%, 97%, 97.5%, 98%, 98.5%, 99%, 99.1%, 99.2%, 99.3%, 99.4%, 99.5%, 99.6%, 99.7%, 99.8%, 99.9% or 100% identical to SEQ ID NO: 7. Preferably, a non-naturally occurring or recombinant polypeptide consists of SEQ ID NO: 7.

Also provided are antibodies or antigen-binding fragments thereof that specifically bind to a non-naturally occurring polypeptide of the application. In an embodiment of the application, an antibody specific to a non-naturally occurring HBV antigen of the application does not bind specifically to another HBV antigen. For example, an antibody of the application that binds specifically to an HBV Pol antigen having the amino acid sequence of SEQ ID NO: 7 will not bind specifically to an HBV Pol antigen not having the amino acid sequence of SEQ ID NO: 7.

As used herein, the term “antibody” is meant in a broad sense and includes immunoglobulin molecules including polyclonal antibodies, monoclonal antibodies including murine, human, humanized and chimeric monoclonal antibodies, antigen-binding fragments, bispecific or multispecific antibodies, dimeric, tetrameric or multimeric antibodies, Fv, Fab and F(ab′)2, bifunctional hybrid (e.g., Lanzavecchia et al., Eur. J. Immunol. 17:105, 1987), single-chain (Huston et al., Proc. Natl. Acad. Sci. USA 85:5879, 1988; Bird et al., Science 242:423, 1988), antibodies with altered constant regions (e.g., U.S. Pat. No. 5,624,821), domain antibodies and any other modified configuration of the immunoglobulin molecule that comprises an antigen binding site of the required specificity.

As used herein, an antibody that “specifically binds to” an antigen refers to an antibody that binds to the antigen with a KD of 1×10⁻⁷ M or less. Preferably, an antibody that “specifically binds to” an antigen binds to the antigen with a KD of 1×10⁻⁸ M or less, more preferably 5×10⁻⁹ M or less, 1×10⁻⁹ M or less, 5×10⁻¹⁰ M or less, or 1×1¹⁰ M or less. The term “KD” refers to the dissociation constant, which is obtained from the ratio of Kd to Ka (i.e., Kd/Ka) and is expressed as a molar concentration (M). KD values for antibodies can be determined using methods in the art in view of the present disclosure. For example, the KD of an antibody can be determined by using surface plasmon resonance, such as by using a biosensor system, e.g., a Biacore® system, or by using bio-layer interferometry technology, such as an Octet RED96 system. The smaller the value of the KD of an antibody, the higher affinity that the antibody binds to a target antigen.

### Anti-PD-1 and Anti-PD-L1 Antibodies

The application also relates to therapeutic applications of anti-PD-1 or anti-PD-L1 antibodies. As described above, “antibodies” is meant in a broad sense and includes immunoglobulin molecules including polyclonal antibodies, monoclonal antibodies including murine, human, humanized and chimeric monoclonal antibodies, antigen-binding fragments, bispecific or multispecific antibodies, dimeric antibodies, tetrameric antibodies, multimeric antibodies, Fv, Fab, F(ab′)2, bifunctional hybrid antibodies, single-chain antibodies, antibodies with altered constant regions, domain antibodies and any other modified configuration of the immunoglobulin molecule that comprises an antigen binding site of the required specificity.

“Full length antibodies” are comprised of two heavy (H) chains and two light (L) chains inter-connected by disulfide bonds as well as multimers thereof (for example IgM). Each heavy chain is comprised of a heavy chain variable region (VH) and a heavy chain constant region (comprised of domains CH1, hinge CH2 and CH3). Each light chain is comprised of a light chain variable region (VL) and a light chain constant region (CL). The VH and the VL regions can be further subdivided into regions of hypervariability, termed complementarity determining regions (CDR), interspersed with framework regions (FR). Each VH and VL is composed of three CDRs and four FR segments, arranged from amino-terminus to carboxy-terminus in the following order: FR1, CDR1, FR2, CDR2, FR3, CDR3, and FR4.

“Complementarity determining regions (CDR)” are “antigen binding sites” in an antibody. CDRs can be defined using various terms: (i) Complementarity Determining Regions (CDRs), three in the VH (HCDR1, HCDR2, HCDR3) and three in the VL (LCDR1, LCDR2, LCDR3) are based on sequence variability (Wu and Kabat, (1970) J Exp Med 132:211-50; Kabat et al., Sequences of Proteins of Immunological Interest, 5th Ed. Public Health Service, National Institutes of Health, Bethesda, Md., 1991). (ii) “Hypervariable regions”, “HVR”, or “HV”, three in the VH (H1, H2, H3) and three in the VL (L1, L2, L3) refer to the regions of an antibody variable domains which are hypervariable in structure as defined by Chothia and Lesk (Chothia and Lesk, (1987) Mol Biol 196:901-17). The International ImMunoGeneTics (IMGT) database (http://www_imgt_org) provides a standardized numbering and definition of antigen-binding sites. The correspondence between CDRs, HVs and IMGT delineations is described in Lefranc et al., (2003) Dev Comparat Immunol 27:55-77. The term “CDR”, “HCDR1”, “HCDR2”, “HCDR3”, “LCDR1”, “LCDR2” and “LCDR3” as used herein includes CDRs defined by any of the methods described supra, Kabat, Chothia or IMGT, unless otherwise explicitly stated in the specification.

Immunoglobulins can be assigned to five major classes, IgA, IgD, IgE, IgG and IgM, depending on the heavy chain constant domain amino acid sequence. IgA and IgG are further sub-classified as the isotypes IgA1, IgA2, IgG1, IgG2, IgG3 and IgG4. Antibody light chains of any vertebrate species can be assigned to one of two clearly distinct types, namely kappa (κ) and lambda (λ), based on the amino acid sequences of their constant domains.

As used herein, “antibody fragments” or “antigen-binding fragment” refers to a portion of an immunoglobulin molecule that retains the antigen binding properties of the parental full length antibody. Exemplary antigen-binding fragments are heavy chain complementarity determining regions (HCDR) 1, 2 and 3, light chain complementarity determining regions (LCDR) 1, 2 and 3, a heavy chain variable region (VH), a light chain variable region (VL), Fab, F(ab′)2, Fd and Fv fragments as well as domain antibodies (dAb) consisting of either one VH or VL domain. VH and VL domains can be linked together via a synthetic linker to form various types of single chain antibody designs where the VH/VL domains can pair intramolecularly, or intermolecularly in those cases when the VH and VL domains are expressed by separate single chain antibody constructs, to form a monovalent antigen binding site, such as single chain Fv (scFv) or diabody; described for example in Int. Patent Publ. Nos. WO1998/44001, WO1988/01649, WO1994/13804 and WO1992/01047.

As used herein, “monoclonal antibody” refers to an antibody population with single amino acid composition in each heavy and each light chain, except for possible well-known alterations such as removal of C-terminal lysine from the antibody heavy chain. Monoclonal antibodies typically bind one antigenic epitope, except that multispecific monoclonal antibodies bind two or more distinct antigens or epitopes. Bispecific monoclonal antibodies bind two distinct antigenic epitopes. Monoclonal antibodies can have heterogeneous glycosylation within the antibody population. Monoclonal antibodies can be monospecific or multispecific, or monovalent, bivalent or multivalent. A multispecific antibody, such as a bispecific antibody or a trispecific antibody is included in the term monoclonal antibody.

As used herein, “humanized antibodies” refers to antibodies in which at least one CDR is derived from non-human species and the variable region frameworks are derived from human immunoglobulin sequences. Humanized antibodies can include intentionally introduced mutations in the framework regions so that the framework may not be an exact copy of expressed human immunoglobulin or germline gene sequences.

As used herein, “human antibody” refers to an antibody having heavy and light chain variable regions in which both the framework and all 6 CDRs are derived from sequences of human origin. If the antibody contains a constant region or a portion of the constant region, the constant region also is derived from sequences of human origin. Human antibody comprises heavy or light chain variable regions that are “derived from” sequences of human origin if the variable regions of the antibody are obtained from a system that uses human germline immunoglobulin or rearranged immunoglobulin genes. Such exemplary systems are human immunoglobulin gene libraries displayed on phage, and transgenic non-human animals such as mice or rats carrying human immunoglobulin loci as described herein. “Human antibody” can contain amino acid differences when compared to the human germline immunoglobulin or rearranged immunoglobulin genes due to for example naturally occurring somatic mutations or intentional introduction of substitutions into the framework or antigen binding site, or both. Typically, “human antibody” is at least about 80%, 81%, 82%, 83%, 84%, 85%, 86%, 87%, 88%, 89%, 90%, 91%, 92%, 93%, 94%, 95%, 96%, 97%, 98%, 99% or 100% identical in amino acid sequence to an amino acid sequence encoded by human germline immunoglobulin or rearranged immunoglobulin genes. In some cases, “human antibody” can contain consensus framework sequences derived from human framework sequence analyses, for example as described in Knappik et al., (2000) J Mol Biol 296:57-86, or synthetic HCDR3 incorporated into human immunoglobulin gene libraries displayed on phage, for example as described in Shi et al., (2010) J Mol Biol 397:385-96, and in Int. Patent Publ. No. WO2009/085462.

Human antibodies derived from human immunoglobulin sequences can be generated using systems such as phage display incorporating synthetic CDRs and/or synthetic frameworks, or can be subjected to in vitro mutagenesis to improve antibody properties, resulting in antibodies that are not expressed by the human antibody germline repertoire in vivo.

As used herein, “epitope” refers to a portion of an antigen to which an antibody specifically binds. Epitopes typically consist of chemically active (such as polar, nonpolar or hydrophobic) surface groupings of moieties such as amino acids or polysaccharide side chains and can have specific three-dimensional structural characteristics, as well as specific charge characteristics. An epitope can be composed of contiguous and/or discontiguous amino acids that form a conformational spatial unit. For a discontiguous epitope, amino acids from differing portions of the linear sequence of the antigen come in close proximity in 3-dimensional space through the folding of the protein molecule. Antibody “epitope” depends on the methodology used to identify the epitope.

As used herein, “multispecific” refers to an antibody that specifically binds at least two distinct antigens or two distinct epitopes within the antigens, for example three, four or five distinct antigens or epitopes.

As used herein, “bispecific” refers to an antibody that specifically binds two distinct antigens or two distinct epitopes within the same antigen.

As used herein, “PD-1” refers to the programmed death-1 protein, a T-cell co-inhibitor, also known as CD279 or PDCD1. A representative amino acid sequence of full-length PD-1 is provided in GenBank as accession number NP 005009.2. The term “PD-1” includes protein variants and recombinant PD-1 or a fragment thereof. “PD-1” in the application refers to human mature PD-1, unless explicitly stated to the contrary.

PD-1 is a 288 amino acid protein receptor expressed on activated T-cells and B-cells, natural killer cells and monocytes. PD-1 is a member of the CD28/CTLA-4 (cytotoxic T lymphocyte antigen)/ICOS (inducible co-stimulator) family of T-cell co-inhibitory receptors (Chen et al. 2013, Nat. Rev. Immunol. 13: 227-242). The protein has an extracellular N-terminal domain which is IgV-like, a transmembrane domain and an intracellular domain containing an immunoreceptor tyrosine-based inhibitory (ITIM) motif and an immunoreceptor tyrosine-based switch (ITSM) motif (Chattopadhyay et al 2009, Immunol. Rev.). The primary function of PD-1 is to attenuate the immune response (Riley 2009, Immunol. Rev. 229: 114-125). PD-1 has two ligands, PD-Ligand 1 (PD-L1) and PD-L2. PD-L1 (CD274, B7H 1) is expressed widely on both lymphoid and non-lymphoid tissues such as CD4 and CD8 T-cells, macrophage lineage cells, peripheral tissues as well as on tumor cells, virally-infected cells and autoimmune tissue cells. PD-L2 (CD273, B7-DC) has a more restricted expression than PD-L1, being expressed on activated dendritic cells and macrophages (Dong et al 1999, Nature Med.). PD-1 binding to its ligands results in decreased T-cell proliferation and cytokine secretion, compromising humoral and cellular immune responses.

As used herein, “PD-L1” refers to programmed death-ligand 1, also known as CD274 and B7H1. The amino acid sequence of full-length PD-L1 is provided in GenBank as accession number NP_054862.1. The term “PD-L1” also includes protein variants and recombinant PD-L1 or a fragment thereof. Unless specified as being from a non-human species, the term “PD-L1” means human PD-L1.

PD-L1 is a 290 amino acid protein with extracellular IgV-like and IgC-like domains (amino acids 19-239 of full length PD-L1), a transmembrane domain and an intracellular domain of approximately 30 amino acids. PD-L1 is constitutively expressed on many cells such as antigen presenting cells (e.g., dendritic cells, macrophages, and B-cells) and on hematopoietic and non-hematopoietic cells (e.g., vascular endothelial cells, pancreatic islets, and sites of immune privilege). PD-L1 is also expressed on a wide variety of tumors, and virally-infected cells and is a component of the immunosuppressive milieu (Ribas 2012, NEJM 366: 2517-2519). PD-L1 binds to one of two T-cell co-inhibitors PD-1 and B7-1.

T-cell co-stimulatory and co-inhibitory molecules (collectively named co-signaling molecules) play a crucial role in regulating T-cell activation, subset differentiation, effector function and survival (Chen et al 2013, Nature Rev. Immunol. 13: 227-242). Following recognition of cognate peptide-MHC complexes on antigen-presenting cells by the T-cell receptor, co-signaling receptors co-localize with T-cell receptors at the immune synapse, where they synergize with TCR signaling to promote or inhibit T-cell activation and function (Flies et al. 2011, Yale J. Biol. Med. 84: 409-421). The ultimate immune response is regulated by a balance between co-stimulatory and co-inhibitory signals (“immune checkpoints”) (Pardoll 2012, Nature 12: 252-264). While not wishing to be bound by theory, it is currently believed that PD-1 functions as one such ‘immune checkpoint’ in mediating peripheral T-cell tolerance and in avoiding autoimmunity. PD-1 binds to PD-L1 or PD-L2 and inhibits T-cell activation. The ability of PD-1 to inhibit T-cell activation is exploited by chronic viral infections and tumors to evade immune response. In chronic viral infections, PD-1 is highly expressed on virus-specific T-cells and these T-cells become “exhausted” with loss of effector functions and proliferative capacity (Freeman 2008, PNAS 105: 10275-1 0276). The PD-1:PD-L1 system also plays an important role in induced T-regulatory (Treg) cell development and in sustaining Treg function (Francisco et al 2010, Immunol. Rev. 236:219-242).

As used herein, “antagonist” refers to a molecule that, when bound to a cellular protein, suppresses at least one reaction or activity that is induced by a natural ligand of the protein. A molecule is an antagonist when the at least one reaction or activity is suppressed by at least about 30%, 40%, 45%, 50%, 55%, 60%, 65%, 70%, 75%, 80%, 85%, 90%, 95%, or 100% more than the at least one reaction or activity suppressed in the absence of the antagonist (e.g., negative control), or when the suppression is statistically significant when compared to the suppression in the absence of the antagonist. Antagonist can be an antibody, a soluble ligand, a small molecule, a DNA or RNA such as siRNA. Exemplary antagonists are an antagonistic antibody specifically binding PD-1 or to PD-L1. A typical reaction or activity that is induced by PD-1 binding to its receptor PD-L1 or PD-L2 can be reduced antigen-specific CD4+ or CD8+ cell proliferation or reduced interferon-7 (IFN-γ) production by T cells, resulting in suppression of immune responses. Hence, an antagonistic PD-1 antibody specifically binding PD-1, or an anti-PD-L1 antibody specifically binding PD-L1, induces an immune response by inhibiting the inhibitory pathway.

In some embodiments, an anti-PD-1 antibody or antigen-binding fragment thereof has one, two, three, four or five of the following properties:
a) enhances an activation of antigen specific CD4+ or CD8+ T cells in a dose dependent manner, wherein the activation is measured using a cytomegalovirus antigen recall assay (CMV assay) as described in Example 1 of US20170121409, which is incorporated herein by reference in its entirety;
b) binds human PD-1 with an equilibrium dissociation constant (K_{D}) of less than about 100 nM, wherein the K_{D} is measured using ProteOn XPR36 system at +25° C.;
c) binds human PD-1 with the K_{D} of less than about 1 nM, wherein the K_{D} is measured using ProteOn XPR36 system at +25° C.;
d) binds cynomolgus PD-1 with the K_{D} of less than about 100 nM, wherein the K_{D} is measured using ProteOn XPR36 system at +25° C., or
e) binds cynomolgus PD-1 with the K_{D} of less than about 1 nM, wherein the K_{D} is measured using ProteOn XPR36 system at +25° C.

In some embodiments, an anti-PD-L1 antibody or antigen-binding fragment thereof has one, two, three, four or five of the following properties:
a) enhances an activation of antigen specific CD4+ or CD8+ T cells in a dose dependent manner, wherein the activation is measured using a cytomegalovirus antigen recall assay (CMV assay) as described in Example 1 of US20170121409, which is incorporated herein by reference in its entirety;
b) binds human PD-L1 with an equilibrium dissociation constant (K_{D}) of less than about 100 nM, wherein the K_{D} is measured using ProteOn XPR36 system at +25° C.;
c) binds human PD-L1 with the K_{D} of less than about 1 nM, wherein the K_{D} is measured using ProteOn XPR36 system at +25° C.;
d) binds cynomolgus PD-L1 with the K_{D} of less than about 100 nM, wherein the K_{D} is measured using ProteOn XPR36 system at +25° C., or
e) binds cynomolgus PD-L1 with the K_{D} of less than about 1 nM, wherein the K_{D} is measured using ProteOn XPR36 system at +25° C.

The affinity of an antibody to human or cynomolgus PD-1 or PD-L1 can be determined experimentally using any suitable method. Such methods can utilize ProteOn XPR36, Biacore 3000 or KinExA instrumentation, ELISA or competitive binding assays known to those skilled in the art. The measured affinity of a particular antibody/PD-1 or antibody/PD-L1 interaction can vary if measured under different conditions (e.g., osmolarity, pH). Thus, measurements of affinity and other binding parameters (e.g., K_{D}, Kₒₙ, K_{off}) are typically made with standardized conditions and a standardized buffer.

Persons skilled in the art will appreciate that the internal error for affinity measurements for example using Biacore 3000 or ProteOn (measured as standard deviation, SD) can typically be within 5-33% for measurements within the typical limits of detection. Therefore the term “about” in the context of K_{D} reflects the typical standard deviation in the assay. For example, the typical SD for a K_{D} of 1×10⁻⁹M is up to +0.33×10⁻⁹M.

Activation of antigen specific CD4+ or CD8+ T cells can be assessed by measuring increased T cell proliferation in a Mixed Lymphocyte Reaction (MLR) assay, increased interferon-γ (IFN-γ) secretion in the MLR assay, increased TNF-α secretion in the MLR assay, increased IFN-7 secretion in a cytomegalovirus antigen assay (CMV assay) or increased TNF-α secretion in the CMV assay using known protocols and those described in Example 1 of US20170121409. Antibodies of the application enhance the activation of antigen specific CD4+ or CD8+T when the measured T cell functionality is increased by antibodies of the application in a dose-dependent manner.

Any anti-PD-1 or anti-PD-L1 antibodies, and fragments thereof known in the art or to be made in the future can be used in the invention in view of the present disclosure.

In certain embodiments, the anti-PD-1 or anti-PD-L1 antibody, or fragment thereof useful for the invention is Nivolumab (MDX-1 106, Opdivo; Bristol-Myers Squibb), Pembrolizumab (MK-3475, Keytruda, lambrolizumab, BMS-936558; Merck), TSR-042 (Tesaro, Inc.), REGN2810 (Regeneron Pharmaceuticals), EH12.2H7 (BioLegend, catalog no. 329902), Avelumab (Bavencio; EMID Serono, Pfizer), Durvalumab (Imfinzi, AstraZeneca), Cemiplimab (REGN-2810, Libtayo; Regeneron), BMS-936559, or Atezolizumab (Tecentriq, Genentech).

In certain embodiments, the anti-PD-1 or anti-PD-L1 antibody, or fragment thereof useful for the invention, is described in US20020110836, US20030044768, US20050180969, US20060110383, US20060210567, US20070065427, US20070122378, US20080025979, US20080044837, US20090028857, US20090055944, US20100040614, US20100055102, US20100151492, US20100266617, US20110008369, US20110085970, US20110117085, US20110171215, US20110171220, US20110177088, US20110195068, US20110229461, US20110271358, US20120237522, US20120039906, US20130017199, US20130022595, US20130095098, US20140044738, US20150079109, US20160075783, US20170210806, US20170121409, or WO2018039131, the content of each of the prior publications is incorporated herein by referent in its entirety.

In certain embodiments, the anti-PD-1 or anti-PD-L1 antibody, or fragment thereof useful for the invention is described in US20090217401 (e.g., 17D8, 2D3, 4H1, 5C4, 4A11, 7D3, 5F4, and variants thereof), US20140234296 (e.g., H409A11, hPD-1.08A, hPD-1.09A, 109A-H, K09A-L-11, K09A-L-16, K09A-L-17, 409A-H, and variants thereof), US20150203579 (e.g., H1M7789N, H1M7799N, H1M7800N, H2M7780N, H2M7788N, H2M7790N, H2M7791N, H2M7794N, H2M7795N, H2M7796N, H2M7798N, H4H9019P, H4xH9034P2, H4xH9035P2, H4xH9037P2, H4xH9045P2, H4xH9048P2, H4H9057P2, H4H9068P2, H4xH9119P2, H4xH9120P2, H4xH9128P2, H4xH9135P2, H4xH9145P2, H4xH8992P, H4xH8999P, H4xH9008P, H4H7798N, H4H7795N2, H4H9008P, H4H9048P2, and variants thereof), US20170247454 (e.g., PD1-0103, PD1-0098, PD1-0050, PD1-0069, PD1-0073, PD1-0078, PD1-0102, PD1-0103_01, PD1-0103_02, PD1-0103_03, PD1-0103_04, PD1-0103-0312, PD1-0103-0313, PD1-0103-0314, PD1-0103-0315, and variants thereof), or WO2017025051 (1.7.3 hAb, 1.49.9 hAb, 1.103.11 hAb, 1.103.11-v2 hAb, 1.139.15 hAb, 1.153.7 hAb, and variants thereof), the content of each of the prior publications is incorporated herein by referent in its entirety.

Preferably, the anti-PD-1 or anti-PD-L1 antibody, or fragment thereof useful for the invention is antigen-binding fragment thereof is (a) 17D8, 2D3, 4H1, 5C4, 4A11, 7D3, 5F4, H409A11, hPD-1.08A, hPD-1.09A, 109A-H, K09A-L-11, K09A-L-16, K09A-L-17, 409A-H, H1M7789N, H1M7799N, H1M7800N, H2M7780N, H2M7788N, H2M7790N, H2M7791N, H2M7794N, H2M7795N, H2M7796N, H2M7798N, H4H9019P, H4xH9034P2, H4xH9035P2, H4xH9037P2, H4xH9045P2, H4xH9048P2, H4H9057P2, H4H9068P2, H4xH9119P2, H4xH9120P2, H4xH9128P2, H4xH9135P2, H4xH9145P2, H4xH8992P, H4xH8999P, H4xH9008P, H4H7798N, H4H7795N2, H4H9008P, H4H9048P2, PD1-0103, PD1-0098, PD1-0050, PD1-0069, PD1-0073, PD1-0078, PD1-0102, PD1-0103_01, PD1-0103_02, PD1-0103_03, PD1-0103_04, PD1-0103-0312, PD1-0103-0313, PD1-0103-0314, PD1-0103-0315, 1.7.3 hAb, 1.49.9 hAb, 1.103.11 hAb, 1.103.11-v2 hAb, 1.139.15 hAb, 1.153.7 hAb, or variants thereof, (b) antibody binding to an epitope having a sequence of SEHSI, DPFEL, KLNG, QTSWK, LHFEP, NDNGSY, TTLYVT, or LAAFPEDRSQPGQDCR; or (c) Nivolumab (MDX-1 106, Opdivo; Bristol-Myers Squibb), Pembrolizumab (MK-3475, Keytruda, lambrolizumab, BMS-936558; Merck), TSR-042 (Tesaro, Inc.), REGN2810 (Regeneron Pharmaceuticals), EH12.2H7 (BioLegend, catalog no. 329902), Avelumab (Bavencio; EMD Serono, Pfizer), Durvalumab (Imfinzi, AstraZeneca), Cemiplimab (REGN-2810, Libtayo; Regeneron), BMS-936559, Atezolizumab (Tecentriq, Genentech), or an equivalent thereto.

As used herein, the term “equivalent thereto” referring to an antibody means, e.g., an antibody with substantially identical CDRs, binding to substantially the same epitope and having substantially identical biological activities, to the reference antibody.

Variants of anti-PD-1 or anti-PD-L1 antibodies or antigen-binding fragments thereof described herein are within the scope of the application. For example, variants can comprise one, two, three, four, five, six, seven, eight, nine, ten, eleven, twelve, thirteen, fourteen or fifteen amino acid substitutions in the VH and/or the VL as long as the homologous antibodies retain or have improved functional properties when compared to the parental antibodies. In some embodiments, the sequence identity can be about 90%, 91%, 92%, 93%, 94%, 95%, 96%, 97%, 98% or 99% to a VH or the VL amino acid sequence of the application. Optionally, any variation of the variant compared to the parental antibody is not within the CDRs of the variant.

Also provided are antagonistic antibodies specifically binding PD-1 or PD-L1 or antigen-binding fragments thereof comprising the VH comprising the HCDR1, the HCDR2 and the HCDR3 sequences and the VL comprising the LCDR1, the LCDR2 and the LCDR3 sequences, wherein one or more of the CDR sequences comprise specified amino acid sequences based on the antibodies described herein, or conservative modifications thereof, and wherein the antibodies retain the desired functional properties of the parental antagonistic antibodies specifically binding PD-1 or PD-L1 of the application.

“Conservative modification” refers to amino acid modifications that do not significantly affect or alter the binding characteristics of the antibody containing the amino acid sequences. Conservative modifications include amino acid substitutions, additions and deletions. Conservative substitutions are those in which the amino acid is replaced with an amino acid residue having a similar side chain. The families of amino acid residues having similar side chains are well defined and include amino acids with acidic side chains (for example, aspartic acid, glutamic acid), basic side chains (for example, lysine, arginine, histidine), nonpolar side chains (for example, alanine, valine, leucine, isoleucine, proline, phenylalanine, methionine), uncharged polar side chains (for example, glycine, asparagine, glutamine, cysteine, serine, threonine, tyrosine, tryptophan), aromatic side chains (for example, phenylalanine, tryptophan, histidine, tyrosine), aliphatic side chains (for example, glycine, alanine, valine, leucine, isoleucine, serine, threonine), amide (for example, asparagine, glutamine), beta-branched side chains (for example, threonine, valine, isoleucine) and sulfur-containing side chains (cysteine, methionine). Furthermore, any native residue in the polypeptide can also be substituted with alanine, as has been previously described for alanine scanning mutagenesis (MacLennan et al., Acta Physiol. Scand. Suppl. 643:55-67, 1998; Sasaki et al., Adv. Biophys. 35:1-24, 1998). Amino acid substitutions to antibodies of the application can be made by well-known methods for example by PCR mutagenesis (U.S. Pat. No. 4,683,195). Alternatively, libraries of variants can be generated using known methods, for example using random (NNK) or non-random codons, for example DVK codons, which encode ii amino acids (Ala, Cys, Asp, Glu, Gly, Lys, Asn, Arg, Ser, Tyr, Trp). The resulting antibody variants can be tested for their characteristics using assays described herein.

Methods of making and using antibodies, and fragments thereof, are known in the art. Any such known methods can be used in the context of the present application to make and use antibodies, and fragments thereof, that specifically bind to PD-1 or PD-L1. Methods of making and using anti-PD-1 or anti-PD-L1 antibodies, and fragments thereof, are known in the art and are described, e.g., in US20020110836, US20030044768, US20050180969, US20060110383, US20060210567, US2007/0065427, US2007/0122378, US20080025979, US20080044837, US20090028857, US20090055944, US20090217401, US20100040614, US20100055102, US20100151492, US20100266617, US20110008369, US20110085970, US20110117085, US20110171215, US20110171220, US20110177088, US20110195068, US20110229461, US20110271358, US2012/0237522, US20120039906, US20130017199, US20130022595, US20130095098, US20140234296, US20140044738, US20150079109, US20150203579, US20160075783, US20170210806, US20170247454, US20170121409, WO2017025051 and WO2018039131, all of which are incorporated herein by reference in their entirety.

### Compositions, Therapeutic Combinations, and Vaccines

The application also relates to compositions, therapeutic combinations, more particularly kits, and vaccines comprising one or more HBV antigens, polynucleotides, and/or vectors encoding one or more HBV antigens according to the application, and/or one or more anti-PD1 or anti-PD-L1 antibody or antigen-binding fragment thereof Δny of the HBV antigens, polynucleotides (including RNA and DNA), and/or vectors of the application described herein, and any of the anti-PD-1 or anti-PD-L1 antibodies or antigen-binding fragments thereof of the application described herein, can be used in the compositions, therapeutic combinations or kits, and vaccines of the application.

In an embodiment of the application, a composition comprises an isolated or non-naturally occurring nucleic acid molecule (DNA or RNA) comprising polynucleotide sequence encoding a truncated HBV core antigen consisting of an amino acid sequence that is at least 90% identical to SEQ ID NO: 2 or SEQ ID NO: 4, or an HBV polymerase antigen comprising an amino acid sequence that is at least 90% identical to SEQ ID NO: 7, a vector comprising the isolated or non-naturally occurring nucleic acid molecule, and/or an isolated or non-naturally occurring polypeptide encoded by the isolated or non-naturally occurring nucleic acid molecule.

In an embodiment of the application, a composition comprises an isolated or non-naturally occurring nucleic acid molecule (DNA or RNA) comprising a polynucleotide sequence encoding an HBV Pol antigen comprising an amino acid sequence that is at least 90% identical to SEQ ID NO: 7, preferably 100% identical to SEQ ID NO: 7.

In an embodiment of the application, a composition comprises an isolated or non-naturally occurring nucleic acid molecule (DNA or RNA) encoding a truncated HBV core antigen consisting of an amino acid sequence that is at least 90% identical to SEQ ID NO: 2 or SEQ ID NO: 4, preferably 100% identical to SEQ ID NO: 2 or SEQ ID NO: 4.

In an embodiment of the application, a composition comprises an isolated or non-naturally occurring nucleic acid molecule (DNA or RNA) comprising a polynucleotide sequence encoding a truncated HBV core antigen consisting of an amino acid sequence that is at least 90% identical to SEQ ID NO: 2 or SEQ ID NO: 4, preferably 100% identical to SEQ ID NO: 2 or SEQ ID NO: 4; and an isolated or non-naturally occurring nucleic acid molecule (DNA or RNA) comprising a polynucleotide sequence encoding an HBV Pol antigen comprising an amino acid sequence that is at least 90% identical to SEQ ID NO: 7, preferably 100% identical to SEQ ID NO: 7. The coding sequences for the truncated HBV core antigen and the HBV Pol antigen can be present in the same isolated or non-naturally occurring nucleic acid molecule (DNA or RNA), or in two different isolated or non-naturally occurring nucleic acid molecules (DNA or RNA).

In an embodiment of the application, a composition comprises a vector, preferably a DNA plasmid or a viral vector (such as an adenoviral vector) comprising a polynucleotide encoding a truncated HBV core antigen consisting of an amino acid sequence that is at least 90% identical to SEQ ID NO: 2 or SEQ ID NO: 4, preferably 100% identical to SEQ ID NO: 2 or SEQ ID NO: 4.

In an embodiment of the application, a composition comprises a vector, preferably a DNA plasmid or a viral vector (such as an adenoviral vector), comprising a polynucleotide encoding an HBV Pol antigen comprising an amino acid sequence that is at least 90% identical to SEQ ID NO: 7, preferably 100% identical to SEQ ID NO: 7.

In an embodiment of the application, a composition comprises a vector, preferably a DNA plasmid or a viral vector (such as an adenoviral vector), comprising a polynucleotide encoding a truncated HBV core antigen consisting of an amino acid sequence that is at least 90% identical to SEQ ID NO: 2 or SEQ ID NO: 4, preferably 100% identical to SEQ ID NO: 2 or SEQ ID NO: 4; and a vector, preferably a DNA plasmid or a viral vector (such as an adenoviral vector), comprising a polynucleotide encoding an HBV Pol antigen comprising an amino acid sequence that is at least 90% identical to SEQ ID NO: 7, preferably 100% identical to SEQ ID NO: 7. The vector comprising the coding sequence for the truncated HBV core antigen and the vector comprising the coding sequence for the HBV Pol antigen can be the same vector, or two different vectors.

In an embodiment of the application, a composition comprises a vector, preferably a DNA plasmid or a viral vector (such as an adenoviral vector), comprising a polynucleotide encoding a fusion protein comprising a truncated HBV core antigen consisting of an amino acid sequence that is at least 90% identical to SEQ ID NO: 2 or SEQ ID NO: 4, preferably 100% identical to SEQ ID NO: 2 or SEQ ID NO: 4, operably linked to an HBV Pol antigen comprising an amino acid sequence that is at least 90% identical to SEQ ID NO: 7, preferably 100% identical to SEQ ID NO: 7, or vice versa. Preferably, the fusion protein further comprises a linker that operably links the truncated HBV core antigen to the HBV Pol antigen, or vice versa. Preferably, the linker has the amino acid sequence of (AlaGly)n, wherein n is an integer of 2 to 5.

In an embodiment of the application, a composition comprises an isolated or non-naturally occurring truncated HBV core antigen consisting of an amino acid sequence that is at least 90% identical to SEQ ID NO: 2 or SEQ ID NO: 4, preferably 100% identical to SEQ ID NO: 2 or SEQ ID NO: 4.

In an embodiment of the application, a composition comprises an isolated or non-naturally occurring HBV Pol antigen comprising an amino acid sequence that is at least 90% identical to SEQ ID NO: 7, preferably 100% identical to SEQ ID NO: 7.

In an embodiment of the application, a composition comprises an isolated or non-naturally occurring truncated HBV core antigen consisting of an amino acid sequence that is at least 90% identical to SEQ ID NO: 2 or SEQ ID NO: 4, preferably 100% identical to SEQ ID NO: 2 or SEQ ID NO: 4; and an isolated or non-naturally occurring HBV Pol antigen comprising an amino acid sequence that is at least 90% identical to SEQ ID NO: 7, preferably 100% identical to SEQ ID NO: 7.

In an embodiment of the application, a composition comprises an isolated or non-naturally occurring fusion protein comprising a truncated HBV core antigen consisting of an amino acid sequence that is at least 90% identical to SEQ ID NO: 2 or SEQ ID NO: 14, preferably 100% identical to SEQ ID NO: 2 or SEQ ID NO: 4, operably linked to an HBV Pol antigen comprising an amino acid sequence that is at least 90% identical to SEQ ID NO: 7, preferably 100% identical to SEQ ID NO: 7, or vice versa. Preferably, the fusion protein further comprises a linker that operably links the truncated HBV core antigen to the HBV Pol antigen, or vice versa. Preferably, the linker has the amino acid sequence of (AlaGly)n, wherein n is an integer of 2 to 5.

In an embodiment of the application, a composition comprises an anti-PD-1 or anti-PD-L1 antibody or antigen-binding fragment thereof.

In a preferred embodiment, the anti-PD-1 or anti-PD-L1 antibody, or fragment thereof useful for the invention is Nivolumab (MDX-1 106, Opdivo; Bristol-Myers Squibb), Pembrolizumab (MK-3475, Keytruda, lambrolizumab, BMS-936558; Merck), TSR-042 (Tesaro, Inc.), REGN2810 (Regeneron Pharmaceuticals), EH12.2H7 (BioLegend, catalog no. 329902), Avelumab (Bavencio; EMD Serono, Pfizer), Durvalumab (Imfinzi, AstraZeneca), Cemiplimab (REGN-2810, Libtayo; Regeneron), BMS-936559, or Atezolizumab (Tecentriq, Genentech), or an equivalent thereto.

The application also relates to a therapeutic combination or a kit comprising polynucleotides expressing a truncated HBV core antigen and an HBV pol antigen according to embodiments of the application and/or anti-PD-1 or anti-PD-L1 antibodies or antigen-binding fragments thereof according to embodiments of the application. Any polynucleotides and/or vectors encoding HBV core and pol antigens of the application described herein can be used in the therapeutic combinations or kits of the application, and any anti-PD-1 or anti-PD-L1 antibodies or antigen-binding fragments thereof of the application described herein can be used in the therapeutic combinations or kits of the application.

According to embodiments of the application, a therapeutic combination or kit for use in treating an HBV infection in a subject in need thereof, comprises:

i) at least one of:

a) a truncated HBV core antigen consisting of an amino acid sequence that is at least 95% identical to SEQ ID NO: 2, and

b) a first non-naturally occurring nucleic acid molecule comprising a first polynucleotide sequence encoding the truncated HBV core antigen

c) an HBV polymerase antigen having an amino acid sequence that is at least 90% identical to SEQ ID NO: 7, wherein the HBV polymerase antigen does not have reverse transcriptase activity and RNase H activity, and

d) a second non-naturally occurring nucleic acid molecule comprising a second polynucleotide sequence encoding the HBV polymerase antigen; and

ii) an anti-PD-1 or anti-PD-L1 antibody or antigen-binding fragment thereof.

In a particular embodiment of the application, a therapeutic combination or kit comprises: i) a first non-naturally occurring nucleic acid molecule comprising a first polynucleotide sequence encoding a truncated HBV core antigen consisting of an amino acid sequence that is at least 95% identical to SEQ ID NO: 2; ii) a second non-naturally occurring nucleic acid molecule comprising a second polynucleotide sequence encoding an HBV polymerase antigen having an amino acid sequence that is at least 90% identical to SEQ ID NO: 7, wherein the HBV polymerase antigen does not have reverse transcriptase activity and RNase H activity; and iii) an anti-PD-1 or anti-PD-L1 antibody or antigen-binding fragment thereof.

According to embodiments of the application, the polynucleotides in a vaccine combination or kit can be linked or separate, such that the HBV antigens expressed from such polynucleotides are fused together or produced as separate proteins, whether expressed from the same or different polynucleotides. In an embodiment, the first and second polynucleotides are present in separate vectors, e.g., DNA plasmids or viral vectors, used in combination either in the same or separate compositions, such that the expressed proteins are also separate proteins, but used in combination. In another embodiment, the HBV antigens encoded by the first and second polynucleotides can be expressed from the same vector, such that an HBV core-pol fusion antigen is produced. Optionally, the core and pol antigens can be joined or fused together by a short linker. Alternatively, the HBV antigens encoded by the first and second polynucleotides can be expressed independently from a single vector using a using a ribosomal slippage site (also known as cis-hydrolase site) between the core and pol antigen coding sequences. This strategy results in a bicistronic expression vector in which individual core and pol antigens are produced from a single mRNA transcript. The core and pol antigens produced from such a bicistronic expression vector can have additional N or C-terminal residues, depending upon the ordering of the coding sequences on the mRNA transcript. Examples of ribosomal slippage sites that can be used for this purpose include, but are not limited to, the FA2 slippage site from foot-and-mouth disease virus (FMIDV). Another possibility is that the HBV antigens encoded by the first and second polynucleotides can be expressed independently from two separate vectors, one encoding the HBV core antigen and one encoding the HBV pol antigen.

In a preferred embodiment, the first and second polynucleotides are present in separate vectors, e.g., DNA plasmids or viral vectors. Preferably, the separate vectors are present in the same composition.

According to preferred embodiments of the application, a therapeutic combination or kit comprises a first polynucleotide present in a first vector, a second polynucleotide present in a second vector. The first and second vectors can be the same or different. Preferably the vectors are DNA plasmids.

In a particular embodiment of the application, the first vector is a first DNA plasmid, the second vector is a second DNA plasmid. Each of the first and second DNA plasmids comprises an origin of replication, preferably pUC ORI of SEQ ID NO: 21, and an antibiotic resistance cassette, preferably comprising a codon optimized Kanr gene having a polynucleotide sequence that is at least 90% identical to SEQ ID NO: 23, preferably under control of a bla promoter, for instance the bla promoter shown in SEQ ID NO: 24. Each of the first and second DNA plasmids independently further comprises at least one of a promoter sequence, enhancer sequence, and a polynucleotide sequence encoding a signal peptide sequence operably linked to the first polynucleotide sequence or the second polynucleotide sequence. Preferably, each of the first and second DNA plasmids comprises an upstream sequence operably linked to the first polynucleotide or the second polynucleotide, wherein the upstream sequence comprises, from 5′ end to 3′ end, a promoter sequence of SEQ ID NO: 18 or 19, an enhancer sequence, and a polynucleotide sequence encoding a signal peptide sequence having the amino acid sequence of SEQ ID NO: 9 or 15. Each of the first and second DNA plasmids can also comprise a polyadenylation signal located downstream of the coding sequence of the HBV antigen, such as the bGH polyadenylation signal of SEQ ID NO: 20.

In one particular embodiment of the application, the first vector is a viral vector and the second vector is a viral vector. Preferably, each of the viral vectors is an adenoviral vector, more preferably an Ad26 or Ad35 vector, comprising an expression cassette including the polynucleotide encoding an HBV pol antigen or an truncated HBV core antigen of the application; an upstream sequence operably linked to the polynucleotide encoding the HBV antigen comprising, from 5′ end to 3′ end, a promoter sequence, preferably a CMV promoter sequence of SEQ ID NO: 19, an enhancer sequence, preferably an ApoAI gene fragment sequence of SEQ ID NO: 12, and a polynucleotide sequence encoding a signal peptide sequence, preferably an immunoglobulin secretion signal having the amino acid sequence of SEQ ID NO: 15; and a downstream sequence operably linked to the polynucleotide encoding the HBV antigen comprising a polyadenylation signal, preferably a SV40 polyadenylation signal of SEQ ID NO: 13.

In another preferred embodiment, the first and second polynucleotides are present in a single vector, e.g., DNA plasmid or viral vector. Preferably, the single vector is an adenoviral vector, more preferably an Ad26 vector, comprising an expression cassette including a polynucleotide encoding an HBV pol antigen and a truncated HBV core antigen of the application, preferably encoding an HBV pol antigen and a truncated HBV core antigen of the application as a fusion protein; an upstream sequence operably linked to the polynucleotide encoding the HBV pol and truncated core antigens comprising, from 5′ end to 3′ end, a promoter sequence, preferably a CMV promoter sequence of SEQ ID NO: 19, an enhancer sequence, preferably an ApoAI gene fragment sequence of SEQ ID NO: 12, and a polynucleotide sequence encoding a signal peptide sequence, preferably an immunoglobulin secretion signal having the amino acid sequence of SEQ ID NO: 15; and a downstream sequence operably linked to the polynucleotide encoding the HBV antigen comprising a polyadenylation signal, preferably a SV40 polyadenylation signal of SEQ ID NO: 13.

When a therapeutic combination of the application comprises a first vector, such as a DNA plasmid or viral vector, and a second vector, such as a DNA plasmid or viral vector, the amount of each of the first and second vectors is not particularly limited. For example, the first DNA plasmid and the second DNA plasmid can be present in a ratio of 10:1 to 1:10, by weight, such as 10:1, 9:1, 8:1, 7:1, 6:1, 5:1, 4:1, 3:1, 2:1, 1:1, 1:2, 1:3, 1:4, 1:5, 1:6, 1:7, 1:8, 1:9, or 1:10, by weight. Preferably, the first and second DNA plasmids are present in a ratio of 1:1, by weight. The therapeutic combination of the application can further comprise a third vector encoding a third active agent useful for treating an HBV infection.

Compositions and therapeutic combinations of the application can comprise additional polynucleotides or vectors encoding additional HBV antigens and/or additional HBV antigens or immunogenic fragments thereof, such as an HBsAg, an HBV L protein or HBV envelope protein, or a polynucleotide sequence encoding thereof or anti-PD-1 or anti-PD-L1 antibodies or antigen-binding fragments thereof according to embodiments of the application. However, in particular embodiments, the compositions and therapeutic combinations of the application do not comprise certain antigens.

In a particular embodiment, a composition or therapeutic combination or kit of the application does not comprise a HBsAg or a polynucleotide sequence encoding the HBsAg.

In another particular embodiment, a composition or therapeutic combination or kit of the application does not comprise an HBV L protein or a polynucleotide sequence encoding the HBV L protein.

In yet another particular embodiment of the application, a composition or therapeutic combination of the application does not comprise an HBV envelope protein or a polynucleotide sequence encoding the HBV envelope protein.

Compositions and therapeutic combinations of the application can also comprise a pharmaceutically acceptable carrier. A pharmaceutically acceptable carrier is non-toxic and should not interfere with the efficacy of the active ingredient. Pharmaceutically acceptable carriers can include one or more excipients such as binders, disintegrants, swelling agents, suspending agents, emulsifying agents, wetting agents, lubricants, flavorants, sweeteners, preservatives, dyes, solubilizers and coatings. Pharmaceutically acceptable carriers can include vehicles, such as lipid nanoparticles (LNPs). The precise nature of the carrier or other material can depend on the route of administration, e.g., intramuscular, intradermal, subcutaneous, oral, intravenous, cutaneous, intramucosal (e.g., gut), intranasal or intraperitoneal routes. For liquid injectable preparations, for example, suspensions and solutions, suitable carriers and additives include water, glycols, oils, alcohols, preservatives, coloring agents and the like. For solid oral preparations, for example, powders, capsules, caplets, gelcaps and tablets, suitable carriers and additives include starches, sugars, diluents, granulating agents, lubricants, binders, disintegrating agents and the like. For nasal sprays/inhalant mixtures, the aqueous solution/suspension can comprise water, glycols, oils, emollients, stabilizers, wetting agents, preservatives, aromatics, flavors, and the like as suitable carriers and additives.

Compositions and therapeutic combinations of the application can be formulated in any matter suitable for administration to a subject to facilitate administration and improve efficacy, including, but not limited to, oral (enteral) administration and parenteral injections. The parenteral injections include intravenous injection or infusion, subcutaneous injection, intradermal injection, and intramuscular injection. Compositions of the application can also be formulated for other routes of administration including transmucosal, ocular, rectal, long acting implantation, sublingual administration, under the tongue, from oral mucosa bypassing the portal circulation, inhalation, or intranasal.

In a preferred embodiment of the application, compositions and therapeutic combinations of the application are formulated for parental injection, preferably subcutaneous, intradermal injection, or intramuscular injection, more preferably intramuscular injection.

According to embodiments of the application, compositions and therapeutic combinations for administration will typically comprise a buffered solution in a pharmaceutically acceptable carrier, e.g., an aqueous carrier such as buffered saline and the like, e.g., phosphate buffered saline (PBS). The compositions and therapeutic combinations can also contain pharmaceutically acceptable substances as required to approximate physiological conditions such as pH adjusting and buffering agents. For example, a composition or therapeutic combination of the application comprising plasmid DNA can contain phosphate buffered saline (PBS) as the pharmaceutically acceptable carrier. The plasmid DNA can be present in a concentration of, e.g., 0.5 mg/mL to 5 mg/mL, such as 0.5 mg/mL 1, mg/mL, 2 mg/mL, 3 mg/mL, 4 mg/mL, or 5 mg/mL, preferably at 1 mg/mL.

Compositions and therapeutic combinations of the application can be formulated as a vaccine (also referred to as an “immunogenic composition”) according to methods well known in the art. Such compositions can include adjuvants to enhance immune responses. The optimal ratios of each component in the formulation can be determined by techniques well known to those skilled in the art in view of the present disclosure.

In a particular embodiment of the application, a composition or therapeutic combination is a DNA vaccine. DNA vaccines typically comprise bacterial plasmids containing a polynucleotide encoding an antigen of interest under control of a strong eukaryotic promoter. Once the plasmids are delivered to the cell cytoplasm of the host, the encoded antigen is produced and processed endogenously. The resulting antigen typically induces both humoral and cell-medicated immune responses. DNA vaccines are advantageous at least because they offer improved safety, are temperature stable, can be easily adapted to express antigenic variants, and are simple to produce. Any of the DNA plasmids of the application can be used to prepare such a DNA vaccine.

In other particular embodiments of the application, a composition or therapeutic combination is an RNA vaccine. RNA vaccines typically comprise at least one single-stranded RNA molecule encoding an antigen of interest, e.g., a fusion protein or HBV antigen according to the application. Once the RNA is delivered to the cell cytoplasm of the host, the encoded antigen is produced and processed endogenously, inducing both humoral and cell-mediated immune responses, similar to a DNA vaccine. The RNA sequence can be codon optimized to improve translation efficiency. The RNA molecule can be modified by any method known in the art in view of the present disclosure to enhance stability and/or translation, such by adding a polyA tail, e.g., of at least 30 adenosine residues; and/or capping the 5-end with a modified ribonucleotide, e.g., 7-methylguanosine cap, which can be incorporated during RNA synthesis or enzymatically engineered after RNA transcription. An RNA vaccine can also be self-replicating RNA vaccine developed from an alphavirus expression vector. Self-replicating RNA vaccines comprise a replicase RNA molecule derived from a virus belonging to the alphavirus family with a subgenomic promoter that controls replication of the fusion protein or HBV antigen RNA followed by an artificial poly A tail located downstream of the replicase.

In certain embodiments, a further adjuvant can be included in a composition or therapeutic combination of the application, or co-administered with a composition or therapeutic combination of the application. Use of another adjuvant is optional, and can further enhance immune responses when the composition is used for vaccination purposes. Other adjuvants suitable for co-administration or inclusion in compositions in accordance with the application should preferably be ones that are potentially safe, well tolerated and effective in humans. An adjuvant can be a small molecule or antibody including, but not limited to, immune checkpoint inhibitors (e.g., anti-PD-1, anti-TIM-3, etc.), toll-like receptor agonists (e.g., TLR7 agonists and/or TLR8 agonists), RIG-1 agonists, IL-15 superagonists (Altor Bioscience), mutant IRF3 and IRF7 genetic adjuvants, STING agonists (Aduro), FLT3L genetic adjuvant, and IL-7-hyFc. For example, adjuvants can e.g., be chosen from among the following anti-HBV agents: HBV DNA polymerase inhibitors; Immunomodulators; Toll-like receptor 7 modulators; Toll-like receptor 8 modulators; Toll-like receptor 3 modulators; Interferon alpha receptor ligands; Hyaluronidase inhibitors; Modulators of IL-10; HBsAg inhibitors; Toll like receptor 9 modulators; Cyclophilin inhibitors; HBV Prophylactic vaccines; HBV Therapeutic vaccines; HBV viral entry inhibitors; Antisense oligonucleotides targeting viral mRNA, more particularly anti-HBV antisense oligonucleotides; short interfering RNAs (siRNA), more particularly anti-HBV siRNA; Endonuclease modulators; Inhibitors of ribonucleotide reductase; Hepatitis B virus E antigen inhibitors; HBV antibodies targeting the surface antigens of the hepatitis B virus; HBV antibodies; CCR2 chemokine antagonists; Thymosin agonists; Cytokines, such as IL12; Capsid Assembly Modulators, Nucleoprotein inhibitors (HBV core or capsid protein inhibitors); Nucleic Acid Polymers (NAPs); Stimulators of retinoic acid-inducible gene 1; Stimulators of NOD2; Recombinant thymosin alpha-1; Hepatitis B virus replication inhibitors; PI3K inhibitors; cccDNA inhibitors; immune checkpoint inhibitors, such as PD-L1 inhibitors, PD-1 inhibitors, TIM-3 inhibitors, TIGIT inhibitors, Lag3 inhibitors, CTLA-4 inhibitors; Agonists of co-stimulatory receptors that are expressed on immune cells (more particularly T cells), such as CD27 and CD28; BTK inhibitors; Other drugs for treating HBV; IDO inhibitors; Arginase inhibitors; and KDM5 inhibitors.

In certain embodiments, each of the first and second non-naturally occurring nucleic acid molecules is independently formulated with a lipid nanoparticle (LNP).

The application also provides methods of making compositions and therapeutic combinations of the application. A method of producing a composition or therapeutic combination comprises mixing an isolated polynucleotide encoding an HBV antigen, vector, and/or polypeptide of the application with one or more pharmaceutically acceptable carriers. One of ordinary skill in the art will be familiar with conventional techniques used to prepare such compositions.

### Methods of Inducing an Immune Response or Treating an HBV Infection

The application also provides methods of inducing an immune response against hepatitis B virus (HBV) in a subject in need thereof, comprising administering to the subject an immunogenically effective amount of a composition or immunogenic composition of the application. Any of the compositions and therapeutic combinations of the application described herein can be used in the methods of the application.

As used herein, the term “infection” refers to the invasion of a host by a disease causing agent. A disease causing agent is considered to be “infectious” when it is capable of invading a host, and replicating or propagating within the host. Examples of infectious agents include viruses, e.g., HBV and certain species of adenovirus, prions, bacteria, fungi, protozoa and the like. “HBV infection” specifically refers to invasion of a host organism, such as cells and tissues of the host organism, by HBV.

The phrase “inducing an immune response” when used with reference to the methods described herein encompasses causing a desired immune response or effect in a subject in need thereof against an infection, e.g., an HBV infection. “Inducing an immune response” also encompasses providing a therapeutic immunity for treating against a pathogenic agent, e.g., HBV. As used herein, the term “therapeutic immunity” or “therapeutic immune response” means that the vaccinated subject is able to control an infection with the pathogenic agent against which the vaccination was done, for instance immunity against HBV infection conferred by vaccination with HBV vaccine. In an embodiment, “inducing an immune response” means producing an immunity in a subject in need thereof, e.g., to provide a therapeutic effect against a disease, such as HBV infection. In certain embodiments, “inducing an immune response” refers to causing or improving cellular immunity, e.g., T cell response, against HBV infection. In certain embodiments, “inducing an immune response” refers to causing or improving a humoral immune response against HBV infection. In certain embodiments, “inducing an immune response” refers to causing or improving a cellular and a humoral immune response against HBV infection.

As used herein, the term “protective immunity” or “protective immune response” means that the vaccinated subject is able to control an infection with the pathogenic agent against which the vaccination was done. Usually, the subject having developed a “protective immune response” develops only mild to moderate clinical symptoms or no symptoms at all. Usually, a subject having a “protective immune response” or “protective immunity” against a certain agent will not die as a result of the infection with said agent.

Typically, the administration of compositions and therapeutic combinations of the application will have a therapeutic aim to generate an immune response against HBV after HBV infection or development of symptoms characteristic of HBV infection, e.g., for therapeutic vaccination.

As used herein, “an immunogenically effective amount” or “immunologically effective amount” means an amount of a composition, polynucleotide, vector, or antigen sufficient to induce a desired immune effect or immune response in a subject in need thereof Δn immunogenically effective amount can be an amount sufficient to induce an immune response in a subject in need thereof. An immunogenically effective amount can be an amount sufficient to produce immunity in a subject in need thereof, e.g., provide a therapeutic effect against a disease such as HBV infection. An immunogenically effective amount can vary depending upon a variety of factors, such as the physical condition of the subject, age, weight, health, etc.; the particular application, e.g., providing protective immunity or therapeutic immunity; and the particular disease, e.g., viral infection, for which immunity is desired. An immunogenically effective amount can readily be determined by one of ordinary skill in the art in view of the present disclosure.

In particular embodiments of the application, an immunogenically effective amount refers to the amount of a composition or therapeutic combination which is sufficient to achieve one, two, three, four, or more of the following effects: (i) reduce or ameliorate the severity of an HBV infection or a symptom associated therewith; (ii) reduce the duration of an HBV infection or symptom associated therewith; (iii) prevent the progression of an HBV infection or symptom associated therewith; (iv) cause regression of an HBV infection or symptom associated therewith; (v) prevent the development or onset of an HBV infection, or symptom associated therewith; (vi) prevent the recurrence of an HBV infection or symptom associated therewith; (vii) reduce hospitalization of a subject having an HBV infection; (viii) reduce hospitalization length of a subject having an HBV infection; (ix) increase the survival of a subject with an HBV infection; (x) eliminate an HBV infection in a subject; (xi) inhibit or reduce HBV replication in a subject; and/or (xii) enhance or improve the prophylactic or therapeutic effect(s) of another therapy.

An immunogenically effective amount can also be an amount sufficient to reduce HBsAg levels consistent with evolution to clinical seroconversion; achieve sustained HBsAg clearance associated with reduction of infected hepatocytes by a subject's immune system; induce HBV-antigen specific activated T-cell populations; and/or achieve persistent loss of HBsAg within 12 months. Examples of a target index include lower HBsAg below a threshold of 500 copies of HBsAg international units (IU) and/or higher CD8 counts.

As general guidance, an immunogenically effective amount when used with reference to a DNA plasmid can range from about 0.1 mg/mL to 10 mg/mL of DNA plasmid total, such as 0.1 mg/mL, 0.25 mg/mL, 0.5 mg/mL. 0.75 mg/mL 1 mg/mL, 1.5 mg/mL, 2 mg/mL, 3 mg/mL, 4 mg/mL, 5 mg/mL, 6 mg/mL, 7 mg/mL, 8 mg/mL, 9 mg/mL, or 10 mg/mL. Preferably, an immunogenically effective amount of DNA plasmid is less than 8 mg/mL, more preferably less than 6 mg/mL, even more preferably 3-4 mg/mL. An immunogenically effective amount can be from one vector or plasmid, or from multiple vectors or plasmids. As further general guidance, an immunogenically effective amount when used with reference to a peptide can range from about 10 μg to 1 mg per administration, such as 10, 20, 50, 100, 200, 300, 400, 500, 600, 700, 800, 9000, or 1000 μg per administration. An immunogenically effective amount can be administered in a single composition, or in multiple compositions, such as 1, 2, 3, 4, 5, 6, 7, 8, 9, or 10 compositions (e.g., tablets, capsules or injectables, or any composition adapted to intradermal delivery, e.g., to intradermal delivery using an intradermal delivery patch), wherein the administration of the multiple capsules or injections collectively provides a subject with an immunogenically effective amount. For example, when two DNA plasmids are used, an immunogenically effective amount can be 3-4 mg/mL, with 1.5-2 mg/mL of each plasmid. It is also possible to administer an immunogenically effective amount to a subject, and subsequently administer another dose of an immunogenically effective amount to the same subject, in a so-called prime-boost regimen. This general concept of a prime-boost regimen is well known to the skilled person in the vaccine field. Further booster administrations can optionally be added to the regimen, as needed.

A therapeutic combination comprising two DNA plasmids, e.g., a first DNA plasmid encoding an HBV core antigen and second DNA plasmid encoding an HBV pol antigen, can be administered to a subject by mixing both plasmids and delivering the mixture to a single anatomic site. Alternatively, two separate immunizations each delivering a single expression plasmid can be performed. In such embodiments, whether both plasmids are administered in a single immunization as a mixture of in two separate immunizations, the first DNA plasmid and the second DNA plasmid can be administered in a ratio of 10:1 to 1:10, by weight, such as 10:1, 9:1, 8:1, 7:1, 6:1, 5:1, 4:1, 3:1, 2:1, 1:1, 1:2, 1:3, 1:4, 1:5, 1:6, 1:7, 1:8, 1:9, or 1:10, by weight. Preferably, the first and second DNA plasmids are administered in a ratio of 1:1, by weight.

As general guidance, an immunogenically effective amount when used with reference to an anti-PD-1 or anti-PD-L1 antibody or antigen-binding fragment thereof can range from about 0.005 mg to about 100 mg/kg, e.g. about 0.05 mg to about 30 mg/kg or about 5 mg to about 25 mg/kg, or about 4 mg/kg, about 8 mg/kg, about 16 mg/kg or about 24 mg/kg, or for example about 1, 2, 3, 4, 5, 6, 7, 8, 9 or 10 mg/kg, but can even higher, for example about 15, 16, 17, 18, 19, 20, 21, 22, 23, 24, 25, 30, 40, 50, 60, 70, 80, 90 or 100 mg/kg. A fixed unit dose can also be given, for example, 50, 100, 200, 500 or 1000 mg, or the dose can be based on the patient's surface area, e.g., 500, 400, 300, 250, 200, or 100 mg/m2. Usually between 1 and 8 doses, (e.g., 1, 2, 3, 4, 5, 6, 7 or 8) can be administered to treat the patient, but 9, 10, 11, 12, 13, 14, 15, 16, 17, 18, 19, 20 or more doses can be given.

Administration of antibodies or antigen-binding fragments thereof of the application can be repeated after one day, two days, three days, four days, five days, six days, one week, two weeks, three weeks, one month, five weeks, six weeks, seven weeks, two months, three months, four months, five months, six months or longer. Repeated courses of treatment are also possible, as is chronic administration. The repeated administration can be at the same dose or at a different dose. For example, antibodies or antigen-binding fragments thereof of the application can be administered at 8 mg/kg or at 16 mg/kg at weekly interval for 8 weeks, followed by administration at 8 mg/kg or at 16 mg/kg every two weeks for an additional 16 weeks, followed by administration at 8 mg/kg or at 16 mg/kg every four weeks by intravenous infusion. For example, antibodies or antigen-binding fragments thereof of the application can be provided as a daily dosage in an amount of about 0.1-100 mg/kg, such as 0.5, 0.9, 1.0, 1.1, 1.5, 2, 3, 4, 5, 6, 7, 8, 9, 10, 11, 12, 13, 14, 15, 16, 17, 18, 19, 20, 21, 22, 23, 24, 25, 26, 27, 28, 29, 30, 40, 45, 50, 60, 70, 80, 90 or 100 mg/kg, per day, on at least one of day 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40, or alternatively, at least one of week 1, 2, 3, 4, 5, 6, 7, 8, 9, 10, 11, 12, 13, 14, 15, 16, 17, 18, 19 or 20 after initiation of treatment, or any combination thereof, using single or divided doses of every 24, 12, 8, 6, 4, or 2 hours, or any combination thereof.

Preferably, a subject to be treated according to the methods of the application is an HBV-infected subject, particular a subject having chronic HBV infection. Acute HBV infection is characterized by an efficient activation of the innate immune system complemented with a subsequent broad adaptive response (e.g., HBV-specific T-cells, neutralizing antibodies), which usually results in successful suppression of replication or removal of infected hepatocytes. In contrast, such responses are impaired or diminished due to high viral and antigen load, e.g., HBV envelope proteins are produced in abundance and can be released in sub-viral particles in 1,000-fold excess to infectious virus.

Chronic HBV infection is described in phases characterized by viral load, liver enzyme levels (necroinflammatory activity), HBeAg, or HBsAg load or presence of antibodies to these antigens. cccDNA levels stay relatively constant at approximately 10 to 50 copies per cell, even though viremia can vary considerably. The persistence of the cccDNA species leads to chronicity. More specifically, the phases of chronic HBV infection include: (i) the immune-tolerant phase characterized by high viral load and normal or minimally elevated liver enzymes; (ii) the immune activation HBeAg-positive phase in which lower or declining levels of viral replication with significantly elevated liver enzymes are observed; (iii) the inactive HBsAg carrier phase, which is a low replicative state with low viral loads and normal liver enzyme levels in the serum that may follow HBeAg seroconversion; and (iv) the HBeAg-negative phase in which viral replication occurs periodically (reactivation) with concomitant fluctuations in liver enzyme levels, mutations in the pre-core and/or basal core promoter are common, such that HBeAg is not produced by the infected cell.

As used herein, “chronic HBV infection” refers to a subject having the detectable presence of HBV for more than 6 months. A subject having a chronic HBV infection can be in any phase of chronic HBV infection. Chronic HBV infection is understood in accordance with its ordinary meaning in the field. Chronic HBV infection can for example be characterized by the persistence of HBsAg for 6 months or more after acute HBV infection. For example, a chronic HBV infection referred to herein follows the definition published by the Centers for Disease Control and Prevention (CDC), according to which a chronic HBV infection can be characterized by laboratory criteria such as: (i) negative for IgM antibodies to hepatitis B core antigen (IgM anti-HBc) and positive for hepatitis B surface antigen (HBsAg), hepatitis B e antigen (HBeAg), or nucleic acid test for hepatitis B virus DNA, or (ii) positive for HBsAg or nucleic acid test for HBV DNA, or positive for HBeAg two times at least 6 months apart.

Preferably, an immunogenically effective amount refers to the amount of a composition or therapeutic combination of the application which is sufficient to treat chronic HBV infection.

In some embodiments, a subject having chronic HBV infection is undergoing nucleoside analog (NUC) treatment, and is NUC-suppressed. As used herein, “NUC-suppressed”refers to a subject having an undetectable viral level of HBV and stable alanine aminotransferase (ALT) levels for at least six months. Examples of nucleoside/nucleotide analog treatment include HBV polymerase inhibitors, such as entacavir and tenofovir. Preferably, a subject having chronic HBV infection does not have advanced hepatic fibrosis or cirrhosis. Such subject would typically have a METAVIR score of less than 3 for fibrosis and a fibroscan result of less than 9 kPa. The METAVIR score is a scoring system that is commonly used to assess the extent of inflammation and fibrosis by histopathological evaluation in a liver biopsy of patients with hepatitis B. The scoring system assigns two standardized numbers: one reflecting the degree of inflammation and one reflecting the degree of fibrosis.

It is believed that elimination or reduction of chronic HBV may allow early disease interception of severe liver disease, including virus-induced cirrhosis and hepatocellular carcinoma. Thus, the methods of the application can also be used as therapy to treat HBV-induced diseases. Examples of HBV-induced diseases include, but are not limited to cirrhosis, cancer (e.g., hepatocellular carcinoma), and fibrosis, particularly advanced fibrosis characterized by a METAVIR score of 3 or higher for fibrosis. In such embodiments, an immunogenically effective amount is an amount sufficient to achieve persistent loss of HBsAg within 12 months and significant decrease in clinical disease (e.g., cirrhosis, hepatocellular carcinoma, etc.).

Methods according to embodiments of the application further comprises administering to the subject in need thereof another immunogenic agent (such as another HBV antigen or other antigen) or another anti-HBV agent (such as a nucleoside analog or other anti-HBV agent) in combination with a composition of the application. For example, another anti-HBV agent or immunogenic agent can be a small molecule or antibody including, but not limited to, immune checkpoint inhibitors (e.g., anti-PD-1, anti-TIM-3, etc.), toll-like receptor agonists (e.g., TLR7 agonists and/or TLR8 agonists), RIG-1 agonists, IL-15 superagonists (Altor Bioscience), mutant IRF3 and IRF7 genetic adjuvants, STING agonists (Aduro), FLT3L genetic adjuvant, IL12 genetic adjuvant, IL-7-hyFc; CAR-T which bind HBV env (S-CAR cells); capsid assembly modulators; cccDNA inhibitors, HBV polymerase inhibitors (e.g., entecavir and tenofovir). The one or other anti-HBV active agents can be, for example, a small molecule, an antibody or antigen-binding fragment thereof, a polypeptide, protein, or nucleic acid. The one or other anti-HBV agents can e.g., be chosen from among HBV DNA polymerase inhibitors; Immunomodulators; Toll-like receptor 7 modulators; Toll-like receptor 8 modulators; Toll-like receptor 3 modulators; Interferon alpha receptor ligands; Hyaluronidase inhibitors; Modulators of IL-10; HBsAg inhibitors; Toll like receptor 9 modulators; Cyclophilin inhibitors; HBV Prophylactic vaccines; HBV Therapeutic vaccines; HBV viral entry inhibitors; Antisense oligonucleotides targeting viral mRNA, more particularly anti-HBV antisense oligonucleotides; short interfering RNAs (siRNA), more particularly anti-HBV siRNA; Endonuclease modulators; Inhibitors of ribonucleotide reductase; Hepatitis B virus E antigen inhibitors; HBV antibodies targeting the surface antigens of the hepatitis B virus; HBV antibodies; CCR2 chemokine antagonists; Thymosin agonists; Cytokines, such as IL12; Capsid Assembly Modulators, Nucleoprotein inhibitors (HBV core or capsid protein inhibitors); Nucleic Acid Polymers (NAPs); Stimulators of retinoic acid-inducible gene 1; Stimulators of NOD2; Recombinant thymosin alpha-1; Hepatitis B virus replication inhibitors; PI3K inhibitors; cccDNA inhibitors; immune checkpoint inhibitors, such as PD-L1 inhibitors, PD-1 inhibitors, TIM-3 inhibitors, TIGIT inhibitors, Lag3 inhibitors, and CTLA-4 inhibitors; Agonists of co-stimulatory receptors that are expressed on immune cells (more particularly T cells), such as CD27, CD28; BTK inhibitors; Other drugs for treating HBV; IDO inhibitors; Arginase inhibitors; and KDM5 inhibitors.

### Methods of Delivery

Compositions and therapeutic combinations of the application can be administered to a subject by any method known in the art in view of the present disclosure, including, but not limited to, parenteral administration (e.g., intramuscular, subcutaneous, intravenous, or intradermal injection), oral administration, transdermal administration, and nasal administration. Preferably, compositions and therapeutic combinations are administered parenterally (e.g., by intramuscular injection or intradermal injection) or transdermally.

In some embodiments of the application in which a composition or therapeutic combination comprises one or more DNA plasmids, administration can be by injection through the skin, e.g., intramuscular or intradermal injection, preferably intramuscular injection. Intramuscular injection can be combined with electroporation, i.e., application of an electric field to facilitate delivery of the DNA plasmids to cells. As used herein, the term “electroporation” refers to the use of a transmembrane electric field pulse to induce microscopic pathways (pores) in a bio-membrane. During in vivo electroporation, electrical fields of appropriate magnitude and duration are applied to cells, inducing a transient state of enhanced cell membrane permeability, thus enabling the cellular uptake of molecules unable to cross cell membranes on their own. Creation of such pores by electroporation facilitates passage of biomolecules, such as plasmids, oligonucleotides, siRNAs, drugs, etc., from one side of a cellular membrane to the other. In vivo electroporation for the delivery of DNA vaccines has been shown to significantly increase plasmid uptake by host cells, while also leading to mild-to-moderate inflammation at the injection site. As a result, transfection efficiency and immune response are significantly improved (e.g., up to 1,000 fold and 100 fold respectively) with intradermal or intramuscular electroporation, in comparison to conventional injection.

In a typical embodiment, electroporation is combined with intramuscular injection. However, it is also possible to combine electroporation with other forms of parenteral administration, e.g., intradermal injection, subcutaneous injection, etc.

Administration of a composition, therapeutic combination or vaccine of the application via electroporation can be accomplished using electroporation devices that can be configured to deliver to a desired tissue of a mammal a pulse of energy effective to cause reversible pores to form in cell membranes. The electroporation device can include an electroporation component and an electrode assembly or handle assembly. The electroporation component can include one or more of the following components of electroporation devices: controller, current waveform generator, impedance tester, waveform logger, input element, status reporting element, communication port, memory component, power source, and power switch. Electroporation can be accomplished using an in vivo electroporation device. Examples of electroporation devices and electroporation methods that can facilitate delivery of compositions and therapeutic combinations of the application, particularly those comprising DNA plasmids, include CELLECTRA® (Inovio Pharmaceuticals, Blue Bell, Pa.), Elgen electroporator (Inovio Pharmaceuticals, Inc.) Tri-Grid™ delivery system (Ichor Medical Systems, Inc., San Diego, Calif. 92121) and those described in U.S. Pat. Nos. 7,664,545, 8,209,006, 9,452,285, 5,273,525, 6,110,161, 6,261,281, 6,958,060, and 6,939,862, 7,328,064, 6,041,252, 5,873,849, 6,278,895, 6,319,901, 6,912,417, 8,187,249, 9,364,664, 9,802,035, 6,117,660, and International Patent Application Publication WO2017172838, all of which are herein incorporated by reference in their entireties. Other examples of in vivo electroporation devices are described in International Patent Application entitled “Method and Apparatus for the Delivery of Hepatitis B Virus (HBV) Vaccines,” filed on the same day as this application with the Attorney Docket Number 688097-405WO, the contents of which are hereby incorporated by reference in their entireties. Also contemplated by the application for delivery of the compositions and therapeutic combinations of the application are use of a pulsed electric field, for instance as described in, e.g., U.S. Pat. No. 6,697,669, which is herein incorporated by reference in its entirety.

In other embodiments of the application in which a composition or therapeutic combination comprises one or more DNA plasmids, the method of administration is transdermal. Transdermal administration can be combined with epidermal skin abrasion to facilitate delivery of the DNA plasmids to cells. For example, a dermatological patch can be used for epidermal skin abrasion. Upon removal of the dermatological patch, the composition or therapeutic combination can be deposited on the abraised skin.

Methods of delivery are not limited to the above described embodiments, and any means for intracellular delivery can be used. Other methods of intracellular delivery contemplated by the methods of the application include, but are not limited to, liposome encapsulation, lipid nanoparticles (LNPs), etc.

In certain embodiments of the application, the method of administration is a lipid composition, such as a lipid nanoparticle (LNP). Lipid compositions, preferably lipid nanoparticles, that can be used to deliver a therapeutic product (such as one or more nucleic acid molecules of the invention), include, but are not limited to, liposomes or lipid vesicles, wherein an aqueous volume is encapsulated by amphipathic lipid bilayers, or wherein the lipids coat an interior that comprises a therapeutic product; or lipid aggregates or micelles, wherein the lipid-encapsulated therapeutic product is contained within a relatively disordered lipid mixture.

In particular embodiments, the LNPs comprise a cationic lipid to encapsulate and/or enhance the delivery of a nucleic acid molecule, such as a DNA or RNA molecule of the invention, into the target cell. The cationic lipid can be any lipid species that carries a net positive charge at a selected pH, such as physiological pH. The lipid nanoparticles can be prepared by including multi-component lipid mixtures of varying ratios employing one or more cationic lipids, non-cationic lipids and polyethylene glycol (PEG)—modified lipids. Several cationic lipids have been described in the literature, many of which are commercially available. For example, suitable cationic lipids for use in the compositions and methods of the invention include 1,2-dioleoyl-3-trimethylammonium-propane (DOTAP).

The LNP formulations can include anionic lipids. The anionic lipids can be any lipid species that carries a net negative charge at a selected pH, such as physiological pH. The anionic lipids, when combined with cationic lipids, are used to reduce the overall surface charge of LNPs and to introduce pH-dependent disruption of the LNP bilayer structure, facilitating nucleotide release. Several anionic lipids have been described in the literature, many of which are commercially available. For example, suitable anionic lipids for use in the compositions and methods of the invention include 1,2-dioleoyl-sn-glycero-3-phosphoethanolamine (DOPE).

LNPs can be prepared using methods well known in the art in view of the present disclosure. For example, the LNPs can be prepared using ethanol injection or dilution, thin film hydration, freeze-thaw, French press or membrane extrusion, diafiltration, sonication, detergent dialysis, ether infusion, and reverse phase evaporation.

Some examples of lipids, lipid compositions, and methods to create lipid carriers for delivering active nucleic acid molecules, such as those of this invention, are described in: US2017/0190661, US2006/0008910, US2015/0064242, US2005/0064595, WO/2019/036030, US2019/0022247, WO/2019/036028, WO/2019/036008, WO/2019/036000, US2016/0376224, US2017/0119904, WO/2018/200943, WO/2018/191657, US2014/0255472, and US2013/0195968, the relevant content of each of which is hereby incorporated by reference in its entirety.

Anti-PD-1 or anti-PD-L1 antibodies or antigen-binding fragments thereof of the application can be administered to a subject by any suitable route, for example parentally by intravenous (i.v.) infusion or bolus injection, intramuscularly or subcutaneously or intraperitoneally. Intravenous infusion can be given over for example 15, 30, 60, 90, 120, 180, or 240 minutes, or from 1, 2, 3, 4, 5, 6, 7, 8, 9, 10, 11 or 12 hours.

### Adjuvants

In some embodiments of the application, a method of inducing an immune response against HBV further comprises administering an adjuvant. The terms “adjuvant” and “immune stimulant” are used interchangeably herein, and are defined as one or more substances that cause stimulation of the immune system. In this context, an adjuvant is used to enhance an immune response to HBV antigens and antigenic HBV polypeptides of the application.

According to embodiments of the application, an adjuvant can be present in a therapeutic combination or composition of the application, or administered in a separate composition. An adjuvant can be, e.g., a small molecule or an antibody. Examples of adjuvants suitable for use in the application include, but are not limited to, immune checkpoint inhibitors (e.g., anti-PD-1, anti-TIM-3, etc.), toll-like receptor agonists (e.g., TLR7 and/or TLR8 agonists), RIG-1 agonists, IL-15 superagonists (Altor Bioscience), mutant IRF3 and IRF7 genetic adjuvants, STING agonists (Aduro), FLT3L genetic adjuvant, IL12 genetic adjuvant, and IL-7-hyFc. Examples of adjuvants can e.g., be chosen from among the following anti-HBV agents: HBV DNA polymerase inhibitors; Immunomodulators; Toll-like receptor 7 modulators; Toll-like receptor 8 modulators; Toll-like receptor 3 modulators; Interferon alpha receptor ligands; Hyaluronidase inhibitors; Modulators of IL-10; HBsAg inhibitors; Toll like receptor 9 modulators; Cyclophilin inhibitors; HBV Prophylactic vaccines; HBV Therapeutic vaccines; HBV viral entry inhibitors; Antisense oligonucleotides targeting viral mRNA, more particularly anti-HBV antisense oligonucleotides; short interfering RNAs (siRNA), more particularly anti-HBV siRNA; Endonuclease modulators; Inhibitors of ribonucleotide reductase; Hepatitis B virus E antigen inhibitors; HBV antibodies targeting the surface antigens of the hepatitis B virus; HBV antibodies; CCR2 chemokine antagonists; Thymosin agonists; Cytokines, such as IL12; Capsid Assembly Modulators, Nucleoprotein inhibitors (HBV core or capsid protein inhibitors); Nucleic Acid Polymers (NAPs); Stimulators of retinoic acid-inducible gene 1; Stimulators of NOD2; Recombinant thymosin alpha-1; Hepatitis B virus replication inhibitors; PI3K inhibitors; cccDNA inhibitors; immune checkpoint inhibitors, such as PD-L1 inhibitors, PD-1 inhibitors, TIM-3 inhibitors, TIGIT inhibitors, Lag3 inhibitors, and CTLA-4 inhibitors; Agonists of co-stimulatory receptors that are expressed on immune cells (more particularly T cells), such as CD27, CD28; BTK inhibitors; Other drugs for treating HBV; IDO inhibitors; Arginase inhibitors; and KDM5 inhibitors.

Compositions and therapeutic combinations of the application can also be administered in combination with at least one other anti-HBV agent. Examples of anti-HBV agents suitable for use with the application include, but are not limited to small molecules, antibodies, and/or CAR-T therapies which bind HBV env (S-CAR cells), capsid assembly modulators, TLR agonists (e.g., TLR7 and/or TLR8 agonists), cccDNA inhibitors, HBV polymerase inhibitors (e.g., entecavir and tenofovir), and/or immune checkpoint inhibitors, etc.

The at least one anti-HBV agent can e.g., be chosen from among HBV DNA polymerase inhibitors; Immunomodulators; Toll-like receptor 7 modulators; Toll-like receptor 8 modulators; Toll-like receptor 3 modulators; Interferon alpha receptor ligands; Hyaluronidase inhibitors; Modulators of IL-10; HBsAg inhibitors; Toll like receptor 9 modulators; Cyclophilin inhibitors; HBV Prophylactic vaccines; HBV Therapeutic vaccines; HBV viral entry inhibitors; Antisense oligonucleotides targeting viral mRNA, more particularly anti-HBV antisense oligonucleotides; short interfering RNAs (siRNA), more particularly anti-HBV siRNA; Endonuclease modulators; Inhibitors of ribonucleotide reductase; Hepatitis B virus E antigen inhibitors; HBV antibodies targeting the surface antigens of the hepatitis B virus; HBV antibodies; CCR2 chemokine antagonists; Thymosin agonists; Cytokines, such as IL12; Capsid Assembly Modulators, Nucleoprotein inhibitors (HBV core or capsid protein inhibitors); Nucleic Acid Polymers (NAPs); Stimulators of retinoic acid-inducible gene 1; Stimulators of NOD2; Recombinant thymosin alpha-1; Hepatitis B virus replication inhibitors; PI3K inhibitors; cccDNA inhibitors; immune checkpoint inhibitors, such as PD-L1 inhibitors, PD-1 inhibitors, TIM-3 inhibitors, TIGIT inhibitors, Lag3 inhibitors, and CTLA-4 inhibitors; Agonists of co-stimulatory receptors that are expressed on immune cells (more particularly T cells), such as CD27, CD28; BTK inhibitors; Other drugs for treating HBV; IDO inhibitors; Arginase inhibitors; and KDM5 inhibitors. Such anti-HBV agents can be administered with the compositions and therapeutic combinations of the application simultaneously or sequentially.

### Methods of Prime/Boost Immunization

Embodiments of the application also contemplate administering an immunogenically effective amount of a composition or therapeutic combination to a subject, and subsequently administering another dose of an immunogenically effective amount of a composition or therapeutic combination to the same subject, in a so-called prime-boost regimen Thus, in an embodiment, a composition or therapeutic combination of the application is a primer vaccine used for priming an immune response. In another embodiment, a composition or therapeutic combination of the application is a booster vaccine used for boosting an immune response. The priming and boosting vaccines of the application can be used in the methods of the application described herein. This general concept of a prime-boost regimen is well known to the skilled person in the vaccine field. Any of the compositions and therapeutic combinations of the application described herein can be used as priming and/or boosting vaccines for priming and/or boosting an immune response against HBV.

In some embodiments of the application, a composition or therapeutic combination of the application can be administered for priming immunization. The composition or therapeutic combination can be re-administered for boosting immunization. Further booster administrations of the composition or vaccine combination can optionally be added to the regimen, as needed. An adjuvant can be present in a composition of the application used for boosting immunization, present in a separate composition to be administered together with the composition or therapeutic combination of the application for the boosting immunization, or administered on its own as the boosting immunization. In those embodiments in which an adjuvant is included in the regimen, the adjuvant is preferably used for boosting immunization.

An illustrative and non-limiting example of a prime-boost regimen includes administering a single dose of an immunogenically effective amount of a composition or therapeutic combination of the application to a subject to prime the immune response; and subsequently administering another dose of an immunogenically effective amount of a composition or therapeutic combination of the application to boost the immune response, wherein the boosting immunization is first administered about two to six weeks, preferably four weeks after the priming immunization is initially administered. Optionally, about 10 to 14 weeks, preferably 12 weeks, after the priming immunization is initially administered, a further boosting immunization of the composition or therapeutic combination, or other adjuvant, is administered.

### Kits

Also provided herein is a kit comprising a therapeutic combination of the application. A kit can comprise the first polynucleotide, the second polynucleotide, and the anti-PD-1 or anti-PD-L1 antibody or antigen-binding fragment thereof in one or more separate compositions, or a kit can comprise the first polynucleotide, the second polynucleotide, and the anti-PD-1 or anti-PD-L1 antibody or antigen-binding fragment thereof in a single composition. A kit can further comprise one or more adjuvants or immune stimulants, and/or other anti-HBV agents.

The ability to induce or stimulate an anti-HBV immune response upon administration in an animal or human organism can be evaluated either in vitro or in vivo using a variety of assays which are standard in the art. For a general description of techniques available to evaluate the onset and activation of an immune response, see for example Coligan et al. (1992 and 1994, Current Protocols in Immunology; ed. J Wiley & Sons Inc, National Institute of Health). Measurement of cellular immunity can be performed by measurement of cytokine profiles secreted by activated effector cells including those derived from CD4+ and CD8+ T-cells (e.g. quantification of IL-10 or IFN gamma-producing cells by ELISPOT), by determination of the activation status of immune effector cells (e.g. T cell proliferation assays by a classical [3H] thymidine uptake or flow cytometry-based assays), by assaying for antigen-specific T lymphocytes in a sensitized subject (e.g. peptide-specific lysis in a cytotoxicity assay, etc.).

The ability to stimulate a cellular and/or a humoral response can be determined by antibody binding and/or competition in binding (see for example Harlow, 1989, Antibodies, Cold Spring Harbor Press). For example, titers of antibodies produced in response to administration of a composition providing an immunogen can be measured by enzyme-linked immunosorbent assay (ELISA). The immune responses can also be measured by neutralizing antibody assay, where a neutralization of a virus is defined as the loss of infectivity through reaction/inhibition/neutralization of the virus with specific antibody. The immune response can further be measured by Antibody-Dependent Cellular Phagocytosis (ADCP) Assay.

### EMBODIMENTS

The invention provides also the following non-limiting embodiments.

Embodiment 1 is a therapeutic combination for use in treating a hepatitis B virus (HBV) infection in a subject in need thereof, comprising:

i) at least one of:
a) a truncated HBV core antigen consisting of an amino acid sequence that is at least 95%, such as at least 95%, 96%, 97%, 98%, 99% or 100%, identical to SEQ ID NO: 2,
b) a first non-naturally occurring nucleic acid molecule comprising a first polynucleotide sequence encoding the truncated HBV core antigen
c) an HBV polymerase antigen having an amino acid sequence that is at least 90%, such as at least 90%, 91%, 92%, 93%, 94%, 95%, 96%, 97%, 98%, 99% or 100%, identical to SEQ ID NO: 7, wherein the HBV polymerase antigen does not have reverse transcriptase activity and RNase H activity, and
d) a second non-naturally occurring nucleic acid molecule comprising a second polynucleotide sequence encoding the HBV polymerase antigen; and

ii) an anti-PD-1 or anti-PD-L1 antibody or antigen-binding fragment thereof, preferably selected from the group consisting of:
a) 17D8, 2D3, 4H1, 5C4, 4A11, 7D3, 5F4, H409A11, hPD-1.08A, hPD-1.09A, 109A-H, K09A-L-11, K09A-L-16, K09A-L-17, 409A-H, H1M7789N, H1M7799N, H1M7800N, H2M7780N, H2M7788N, H2M7790N, H2M7791N, H2M7794N, H2M7795N, H2M7796N, H2M7798N, H4H9019P, H4xH9034P2, H4xH9035P2, H4xH9037P2, H4xH9045P2, H4xH9048P2, H4H9057P2, H4H9068P2, H4xH9119P2, H4xH9120P2, H4xH9128P2, H4xH9135P2, H4xH9145P2, H4xH8992P, H4xH8999P, H4xH9008P, H4H7798N, H4H7795N2, H4H9008P, H4H9048P2, PD1-0103, PD1-0098, PD1-0050, PD1-0069, PD1-0073, PD1-0078, PD1-0102, PD1-0103_01, PD1-0103_02, PD1-0103_03, PD1-0103_04, PD1-0103-0312, PD1-0103-0313, PD1-0103-0314, PD1-0103-0315, 1.7.3 hAb, 1.49.9 hAb, 1.103.11 hAb, 1.103.11-v2 hAb, 1.139.15 hAb, 1.153.7 hAb, and variants thereof;
b) an antibody binding to an epitope having a sequence of SEHSI, DPFEL, KLNG, QTSWK, LHFEP, NDNGSY, TTLYVT, or LAAFPEDRSQPGQDCR; and
c) Nivolumab (MDX-1 106, Opdivo; Bristol-Myers Squibb), Pembrolizumab (MK-3475, Keytruda, lambrolizumab, BMS-936558; Merck), TSR-042 (Tesaro, Inc.), REGN2810 (Regeneron Pharmaceuticals), EH12.2H7 (BioLegend, catalog no. 329902), Avelumab (Bavencio; EMD Serono, Pfizer), Durvalumab (Imfinzi, AstraZeneca), Cemiplimab (REGN-2810, Libtayo; Regeneron), BMS-936559, Atezolizumab (Tecentriq, Genentech), or an equivalent thereto.

Embodiment 2 is the therapeutic combination of embodiment 1, comprising at least one of the HBV polymerase antigen and the truncated HBV core antigen.

Embodiment 3 is the therapeutic combination of embodiment 2, comprising the HBV polymerase antigen and the truncated HBV core antigen.

Embodiment 4 is the therapeutic combination of embodiment 1, comprising at least one of the first non-naturally occurring nucleic acid molecule comprising the first polynucleotide sequence encoding the truncated HBV core antigen, and the second non-naturally occurring nucleic acid molecule comprising the second polynucleotide sequence encoding the HBV polymerase antigen.

Embodiment 5 is a therapeutic combination for use in treating a hepatitis B virus (HBV) infection in a subject in need thereof, comprising
i) a first non-naturally occurring nucleic acid molecule comprising a first polynucleotide sequence encoding a truncated HBV core antigen consisting of an amino acid sequence that is at least 95% identical to SEQ ID NO: 2; and
ii) a second non-naturally occurring nucleic acid molecule comprising a second polynucleotide sequence encoding an HBV polymerase antigen having an amino acid sequence that is at least 90% identical to SEQ ID NO: 7, wherein the HBV polymerase antigen does not have reverse transcriptase activity and RNase H activity; and
iii) an anti-PD-1 or anti-PD-L1 antibody or antigen-binding fragment antigen-binding fragment thereof, preferably selected from the group consisting of:
   a) 17D8, 2D3, 4H1, 5C4, 4A11, 7D3, 5F4, H409A11, hPD-1.08A, hPD-1.09A, 109A-H, K09A-L-11, K09A-L-16, K09A-L-17, 409A-H, H1M7789N, H1M7799N, H1M7800N, H2M7780N, H2M7788N, H2M7790N, H2M7791N, H2M7794N, H2M7795N, H2M7796N, H2M7798N, H4H9019P, H4xH9034P2, H4xH9035P2, H4xH9037P2, H4xH9045P2, H4xH9048P2, H4H9057P2, H4H9068P2, H4xH9119P2, H4xH9120P2, H4xH9128P2, H4xH9135P2, H4xH9145P2, H4xH8992P, H4xH8999P, H4xH9008P, H4H7798N, H4H7795N2, H4H9008P, H4H9048P2, PD1-0103, PD1-0098, PD1-0050, PD1-0069, PD1-0073, PD1-0078, PD1-0102, PD1-0103_01, PD1-0103_02, PD1-0103_03, PD1-0103_04, PD1-0103-0312, PD1-0103-0313, PD1-0103-0314, PD1-0103-0315, 1.7.3 hAb, 1.49.9 hAb, 1.103.11 hAb, 1.103.11-v2 hAb, 1.139.15 hAb, 1.153.7 hAb, and variants thereof;
   b) an antibody binding to an epitope having a sequence of SEHSI, DPFEL, KLNG, QTSWK, LHFEP, NDNGSY, TTLYVT, or LAAFPEDRSQPGQDCR; and
   c) Nivolumab (MDX-1 106, Opdivo; Bristol-Myers Squibb), Pembrolizumab (MK-3475, Keytruda, lambrolizumab, BMS-936558; Merck), TSR-042 (Tesaro, Inc.), REGN2810 (Regeneron Pharmaceuticals), EH12.2H7 (BioLegend, catalog no. 329902), Avelumab (Bavencio; EMD Serono, Pfizer), Durvalumab (Imfinzi, AstraZeneca), Cemiplimab (REGN-2810, Libtayo; Regeneron), BMS-936559, Atezolizumab (Tecentriq, Genentech), or an equivalent thereto.

Embodiment 6 is the therapeutic combination of embodiment 4 or 5, wherein the first non-naturally occurring nucleic acid molecule further comprises a polynucleotide sequence encoding a signal sequence operably linked to the N-terminus of the truncated HBV core antigen.

Embodiment 6a is the therapeutic combination of any one of embodiments 4 to 6, wherein the second non-naturally occurring nucleic acid molecule further comprises a polynucleotide sequence encoding a signal sequence operably linked to the N-terminus of the HBV polymerase antigen.

Embodiment 6b is the therapeutic combination of embodiment 6 or 6a, wherein the signal sequence independently comprises the amino acid sequence of SEQ ID NO: 9 or SEQ ID NO: 15.

Embodiment 6c is the therapeutic combination of embodiment 6 or 6a, wherein the signal sequence is independently encoded by the polynucleotide sequence of SEQ ID NO: 8 or SEQ ID NO: 14.

Embodiment 7 is the therapeutic combination of any one of embodiments 1-6c, wherein the HBV polymerase antigen comprises an amino acid sequence that is at least 98%, such as at least 98%, 98.5%, 99%, 99.1%, 99.2%, 99.3%, 99.4%, 99.5%, 99.6%, 99.7%, 99.8%, 99.9%, or 100%, identical to SEQ ID NO: 7.

Embodiment 7a is the therapeutic combination of embodiment 7, wherein the HBV polymerase antigen comprises the amino acid sequence of SEQ ID NO: 7.

Embodiment 7b is the therapeutic combination of any one of embodiments 1 to 7a, wherein and the truncated HBV core antigen consists of the amino acid sequence that is at least 98%, such as at least 98%, 98.5%, 99%, 99.1%, 99.2%, 99.3%, 99.4%, 99.5%, 99.6%, 99.7%, 99.8%, 99.9%, or 100%, identical to SEQ ID NO: 2.

Embodiment 7c is the therapeutic combination of embodiment 7b, wherein the truncated HBV antigen consists of the amino acid sequence of SEQ ID NO: 2 or SEQ ID NO: 4.

Embodiment 8 is the therapeutic combination of any one of embodiments 1-7c, wherein each of the first and second non-naturally occurring nucleic acid molecules is a DNA molecule.

Embodiment 8a is the therapeutic combination of embodiment 8, wherein the DNA molecule is present on a DNA vector.

Embodiment 8b is the therapeutic combination of embodiment 8a, wherein the DNA vector is selected from the group consisting of DNA plasmids, bacterial artificial chromosomes, yeast artificial chromosomes, and closed linear deoxyribonucleic acid.

Embodiment 8c is the therapeutic combination of embodiment 8, wherein the DNA molecule is present on a viral vector.

Embodiment 8d is the therapeutic combination of embodiment 8c, wherein the viral vector is selected from the group consisting of bacteriophages, animal viruses, and plant viruses.

Embodiment 8e is the therapeutic combination of any one of embodiments 1-7c, wherein each of the first and second non-naturally occurring nucleic acid molecules is an RNA molecule.

Embodiment 8f is the therapeutic combination of embodiment 8e, wherein the RNA molecule is an RNA replicon, preferably a self-replicating RNA replicon, an mRNA replicon, a modified mRNA replicon, or self-amplifying mRNA.

Embodiment 8g is the therapeutic combination of any one of embodiments 1 to 8f, wherein each of the first and second non-naturally occurring nucleic acid molecules is independently formulated with a lipid composition, preferably a lipid nanoparticle (LNP).

Embodiment 9 is the therapeutic combination of any one of embodiments 4-8g, comprising the first non-naturally occurring nucleic acid molecule and the second non-naturally occurring nucleic acid molecule in the same non-naturally occurring nucleic acid molecule.

Embodiment 10 is the therapeutic combination of any one of embodiments 4-8g, comprising the first non-naturally occurring nucleic acid molecule and the second non-naturally occurring nucleic acid molecule in two different non-naturally occurring nucleic acid molecules.

Embodiment 11 is the therapeutic combination of any one of embodiments 4-10, wherein the first polynucleotide sequence comprises a polynucleotide sequence having at least 90%, such as at least 90%, 91%, 92%, 93%, 94%, 95%, 96%, 97%, 98%, 99% or 100%, sequence identity to SEQ ID NO: 1 or SEQ ID NO: 3.

Embodiment 11a is the therapeutic combination of embodiment 11, wherein the first polynucleotide sequence comprises a polynucleotide sequence having at least 98%, such as at least 98%, 98.5%, 99%, 99.1%, 99.2%, 99.3%, 99.4%, 99.5%, 99.6%, 99.7%, 99.8%, 99.9%, or 100%, sequence identity to SEQ ID NO: 1 or SEQ ID NO: 3.

Embodiment 12 is the therapeutic combination of embodiment 11a, wherein the first polynucleotide sequence comprises the polynucleotide sequence of SEQ ID NO: 1 or SEQ ID NO: 3.

Embodiment 13 the therapeutic combination of any one of embodiments 4 to 12, wherein the second polynucleotide sequence comprises a polynucleotide sequence having at least 90%, such as at least 90%, 91%, 92%, 93%, 94%, 95%, 96%, 97%, 98%, 99% or 100%, sequence identity to SEQ ID NO: 5 or SEQ ID NO: 6.

Embodiment 13a the therapeutic combination of embodiment 13, wherein the second polynucleotide sequence comprises a polynucleotide sequence having at least 98%, such as at least 98%, 98.5%, 99%, 99.1%, 99.2%, 99.3%, 99.4%, 99.5%, 99.6%, 99.7%, 99.8%, 99.9%, or 100%, sequence identity to SEQ ID NO: 5 or SEQ ID NO: 6.

Embodiment 14 is the therapeutic combination of embodiment 13a, wherein the second polynucleotide sequence comprises the polynucleotide sequence of SEQ ID NO: 5 or SEQ ID NO: 6.

Embodiment 15 is the therapeutic combination of any one of embodiments 1 to 14, wherein the anti-PD-1 or anti-PD-L1 antibody, or fragment thereof is 17D8, 2D3, 4H1, 5C4, 4A11, 7D3, 5F4, H409A11, hPD-1.08A, hPD-1.09A, 109A-H, K09A-L-11, K09A-L-16, K09A-L-17, 409A-H, H1M7789N, H1M7799N, H1M7800N, H2M7780N, H2M7788N, H2M7790N, H2M7791N, H2M7794N, H2M7795N, H2M7796N, H2M7798N, H4H9019P, H4xH9034P2, H4xH9035P2, H4xH9037P2, H4xH9045P2, H4xH9048P2, H4H9057P2, H4H9068P2, H4xH9119P2, H4xH9120P2, H4xH9128P2, H4xH9135P2, H4xH9145P2, H4xH8992P, H4xH8999P, H4xH9008P, H4H7798N, H4H7795N2, H4H9008P, H4H9048P2, PD1-0103, PD1-0098, PD1-0050, PD1-0069, PD1-0073, PD1-0078, PD1-0102, PD1-0103_01, PD1-0103_02, PD1-0103_03, PD1-0103_04, PD1-0103-0312, PD1-0103-0313, PD1-0103-0314, PD1-0103-0315, 1.7.3 hAb, 1.49.9 hAb, 1.103.11 hAb, 1.103.11-v2 hAb, 1.139.15 hAb, 1.153.7 hAb, or a variant thereof.

Embodiment 15a is the therapeutic combination of any one of embodiments 1 to 14, wherein the anti-PD-1 or anti-PD-L1 antibody, or fragment thereof is an antibody binding to an epitope having a sequence of SEHSI, DPFEL, KLNG, QTSWK, LHFEP, NDNGSY, TTLYVT, or LAAFPEDRSQPGQDCR.

Embodiment 15b is the therapeutic combination of any one of embodiments 1 to 14, wherein the anti-PD-1 or anti-PD-L1 antibody, or fragment thereof is Nivolumab (MDX-1 106, Opdivo; Bristol-Myers Squibb), Pembrolizumab (MK-3475, Keytruda, lambrolizumab, BMS-936558; Merck), TSR-042 (Tesaro, Inc.), REGN2810 (Regeneron Pharmaceuticals), EH12.2H7 (BioLegend, catalog no. 329902), Avelumab (Bavencio; EMID Serono, Pfizer), Durvalumab (Imfinzi, AstraZeneca), Cemiplimab (REGN-2810, Libtayo; Regeneron), BMS-936559, Atezolizumab (Tecentriq, Genentech), or an equivalent thereto.

Embodiment 16 is a kit comprising the therapeutic combination of any one of embodiments 1 to 15b, and instructions for using the therapeutic combination in treating a hepatitis B virus (HBV) infection in a subject in need thereof.

Embodiment 17 is a method of treating a hepatitis B virus (HBV) infection in a subject in need thereof, comprising administering to the subject the therapeutic combination of any one of embodiments 1 to 15b.

Embodiment 17a is the method of embodiment 17, wherein the treatment induces an immune response against a hepatitis B virus in a subject in need thereof, preferably the subject has chronic HBV infection.

Embodiment 17b is the method of embodiment 17 or 17a, wherein the subject has chronic HBV infection.

Embodiment 17c is the method of any one of embodiments 17 to 17b, wherein the subject is in need of a treatment of an HBV-induced disease selected from the group consisting of advanced fibrosis, cirrhosis and hepatocellular carcinoma (HCC).

Embodiment 18 is the method of any one of embodiments 17-17c, wherein the therapeutic combination is administered by injection through the skin, e.g., intramuscular or intradermal injection, preferably intramuscular injection.

Embodiment 19 is the method of embodiment 18, wherein the therapeutic combination comprises at least one of the first and second non-naturally occurring nucleic acid molecules.

Embodiment 19a is the method of embodiment 19, wherein the therapeutic combination comprises the first and second non-naturally occurring nucleic acid molecules.

Embodiment 20 is the method of embodiment 19 or 19a, wherein the non-naturally occurring nucleic acid molecules are administered to the subject by intramuscular injection in combination with electroporation.

Embodiment 21 is the method of embodiment 19 or 19a, wherein the non-naturally occurring nucleic acid molecules are administered to the subject by a lipid composition, preferably by a lipid nanoparticle.

### Examples

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the present description.

### Example 1. HBV Core Plasmid & HBV Pol Plasmid

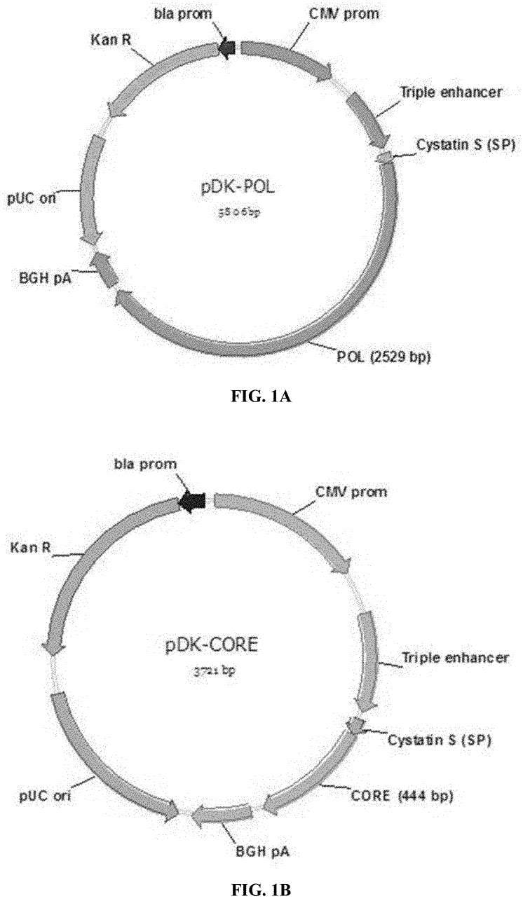

A schematic representation of the pDK-pol and pDK-core vectors is shown in FIGS. 1A and 1B, respectively. An HBV core or pol antigen optimized expression cassette containing a CMV promoter (SEQ ID NO: 18), a splicing enhancer (triple composite sequence) (SEQ ID NO: 10), Cystatin S precursor signal peptide SPCS (NP_0018901.1) (SEQ ID NO: 9), and pol (SEQ ID NO: 5) or core (SEQ ID NO: 2) gene was introduced into a pDK plasmid backbone, using standard molecular biology techniques.

The plasmids were tested in vitro for core and pol antigen expression by Western blot analysis using core and pol specific antibodies, and were shown to provide consistent expression profile for cellular and secreted core and pol antigens (data not shown).

### Example 2. Generation of Adenoviral Vectors Expressing a Fusion of Truncated HBV Core Antigen with HBV Pol Antigen

The creation of an adenovirus vector has been designed as a fusion protein expressed from a single open reading frame. Additional configurations for the expression of the two proteins, e.g. using two separate expression cassettes, or using a 2A-like sequence to separate the two sequences, can also be envisaged.

### Design of Expression Cassettes for Adenoviral Vectors

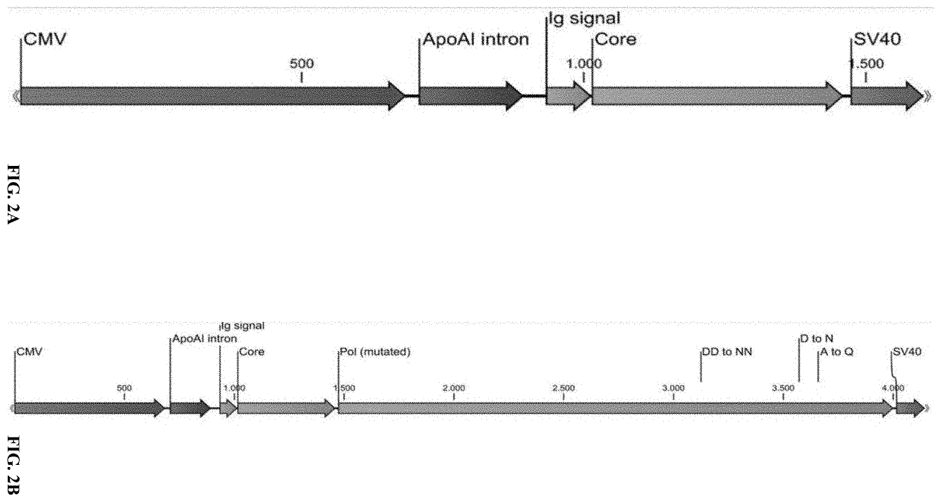

The expression cassettes (diagrammed in FIG. 2A and FIG. 2B) are comprised of the CMV promoter (SEQ ID NO: 19), an intron (SEQ ID NO:12) (a fragment derived from the human ApoAI gene—GenBank accession X01038 base pairs 295-523, harboring the ApoAI second intron), followed by the optimized coding sequence—either core alone or the core and polymerase fusion protein preceded by a human immunoglobulin secretion signal coding sequence (SEQ ID NO: 14), and followed by the SV40 polyadenylation signal (SEQ ID NO: 13).

A secretion signal was included because of past experience showing improvement in the manufacturability of some adenoviral vectors harboring secreted transgenes, without influencing the elicited T-cell response (mouse experiments).

The last two residues of the Core protein (VV) and the first two residues of the Polymerase protein (MP) if fused results in a junction sequence (VVMP) that is present on the human dopamine receptor protein (D3 isoform), along with flanking homologies.

The interjection of an AGAG linker between the core and the polymerase sequences eliminates this homology and returned no further hits in a Blast of the human proteome.

### Example 3. In Vivo Immunogenicity Study of DNA Vaccine in Mice

An immunotherapeutic DNA vaccine containing DNA plasmids encoding an HBV core antigen or HBV polymerase antigen was tested in mice. The purpose of the study was designed to detect T-cell responses induced by the vaccine after intramuscular delivery via electroporation into BALB/c mice. Initial immunogenicity studies focused on determining the cellular immune responses that would be elicited by the introduced HBV antigens.

In particular, the plasmids tested included a pDK-Pol plasmid and pDK-Core plasmid, as shown in FIGS. 1A and 1B, respectively, and as described above in Example 1. The pDK-Pol plasmid encoded a polymerase antigen having the amino acid sequence of SEQ ID NO: 7, and the pDK-Core plasmid encoding a Core antigen having the amino acid sequence of SEQ ID NO: 2. First, T-cell responses induced by each plasmid individually were tested. The DNA plasmid (pDNA) vaccine was intramuscularly delivered via electroporation to Balb/c mice using a commercially available TriGrid™ delivery system-intramuscular (TDS-IM) adapted for application in the mouse model in cranialis tibialis. See International Patent Application Publication WO2017172838, and U.S. Patent Application No. 62/607,430, entitled “Method and Apparatus for the Delivery of Hepatitis B Virus (HBV) Vaccines,” filed on Dec. 19, 2017 for additional description on methods and devices for intramuscular delivery of DNA to mice by electroporation, the disclosures of which are hereby incorporated by reference in their entireties. In particular, the TDS-IM array of a TDS-IM v1.0 device having an electrode array with a 2.5 mm spacing between the electrodes and an electrode diameter of 0.030 inch was inserted percutaneously into the selected muscle, with a conductive length of 3.2 mm and an effective penetration depth of 3.2 mm, and with the major axis of the diamond configuration of the electrodes oriented in parallel with the muscle fibers. Following electrode insertion, the injection was initiated to distribute DNA (e.g., 0.020 ml) in the muscle. Following completion of the IM injection, a 250 V/cm electrical field (applied voltage of 59.4-65.6 V, applied current limits of less than 4 A, 0.16 A/sec) was locally applied for a total duration of about 400 ms at a 10% duty cycle (i.e., voltage is actively applied for a total of about 40 ms of the about 400 ms duration) with 6 total pulses. Once the electroporation procedure was completed, the TriGrid™ array was removed and the animals were recovered. High-dose (20 μg) administration to BALB/c mice was performed as summarized in Table 1. Six mice were administered plasmid DNA encoding the HBV core antigen (pDK-core; Group 1), six mice were administered plasmid DNA encoding the HBV pol antigen (pDK-pol; Group 2), and two mice received empty vector as the negative control. Animals received two DNA immunizations two weeks apart and splenocytes were collected one week after the last immunization.

TABLE 1
Mouse immunization experimental design of the pilot study.

| | | | Unilateral | | | | Endpoint |
|---|---|---|---|---|---|---|---|
| | | | Admin Site | | | | (spleen |
| | | | (alternate | | | Admin | harvest) |
| Group | N | pDNA | sides) | Dose | Vol | Days | Day |
| 1 | 6 | Core | CT + EP | 20 μg | 20 μL | 0, 14 | 21 |
| 2 | 6 | Pol | CT + EP | 20 μg | 20 μL | 0, 14 | 21 |
| 3 | 2 | Empty | CT + EP | 20 μg | 20 μL | 0, 14 | 21 |
| | | Vector | | | | | |
| | | (neg | | | | | |
| | | control) | | | | | |

CT, cranialis tibialis muscle; EP, electroporation.

Antigen-specific responses were analyzed and quantified by IFN-γ enzyme-linked immunospot (ELISPOT). In this assay, isolated splenocytes of immunized animals were incubated overnight with peptide pools covering the Core protein, the Pol protein, or the small peptide leader and junction sequence (2 μg/ml of each peptide). These pools consisted of 15 mer peptides that overlap by 11 residues matching the Genotypes BCD consensus sequence of the Core and Pol vaccine vectors. The large 94 kDan HBV Pol protein was split in the middle into two peptide pools. Antigen-specific T cells were stimulated with the homologous peptide pools and IFN-γ-positive T cells were assessed using the ELISPOT assay. IFN-γ release by a single antigen-specific T cell was visualized by appropriate antibodies and subsequent chromogenic detection as a colored spot on the microplate referred to as spot-forming cell (SFC).

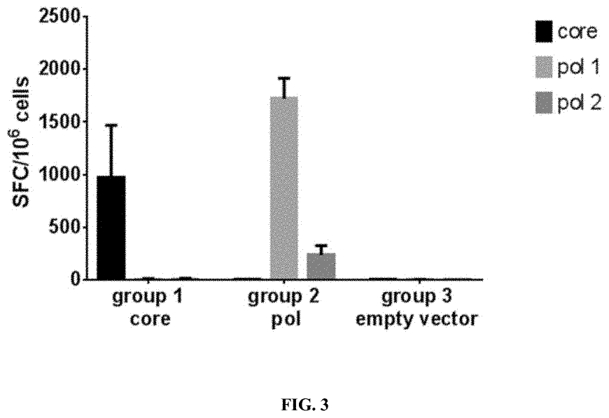

Substantial T-cell responses against HBV Core were achieved in mice immunized with the DNA vaccine plasmid pDK-Core (Group 1) reaching 1,000 SFCs per 10⁶ cells (FIG. 3). Pol T-cell responses towards the Pol 1 peptide pool were strong (˜1,000 SFCs per 10⁶ cells). The weak Pol-2-directed anti-Pol cellular responses were likely due to the limited MHC diversity in mice, a phenomenon called T-cell immunodominance defined as unequal recognition of different epitopes from one antigen. A confirmatory study was performed confirming the results obtained in this study (data not shown).

The above results demonstrate that vaccination with a DNA plasmid vaccine encoding HBV antigens induces cellular immune responses against the administered HBV antigens in mice. Similar results were also obtained with non-human primates (data not shown).

### Example 4. Impact of Anti-PD-1 on HBV Vaccine-Induced Immune Response

To assess whether blocking PD-1 impacts HBV-vaccine induced cell-mediated immunity (CMI) in healthy mice, adult C57BL/6 mice were vaccinated by DNA electroporation at d0 and d21, and their spleens were harvested at d28. The vaccine was a mix of pDF-Core and pDF-Pol vax (same sequences as pDK-Core and pDK-Pol, but different backbone encoding different antibiotic resistance) administered in both legs, 10 μg/each/site. An anti-PD-1 mAb (CD279; InVivomab) or an isotype mAb (InVivomab) was administered by intraperitoneal injection (10 mg/kg) at the time of first vaccination and then once per week until d21 (i.e., d0, d7, d14 and d21). A second group of mice was treated with the anti-PD-1 mAb or isotype mAb at d7, d14 and d21. Control mice were treated with Isotypic antibody.

At day 28, i.e. one week after the last vaccination, mice were sacrificed, and their splenocytes were isolated to measure the immune responses by ELISPOT and intracellular cytokine staining (ICS). Briefly, each spleen was removed from a sacrificed immunized mouse in a sterile manner and placed in a 5 ml snap-cap polypropylene tube containing 2 ml of R10 medium. The spleen was then disrupted in a 6 cm Petri dish by scratching it through a metal grid. The grid was washed with an additional 2 ml of R10 medium, and the sample was collected in a tube. The debris was decanted for few minutes, and the upper phase was quickly transferred to a new tube and centrifuged at 1200 rpm for 10 minutes at room temperature. Red Blood Cell (RBC) osmotic lysis was performed by adding 4.5 ml lysing buffer (ACK Lysing Buffer) to the suspended cell pellet, mixing gently and setting aside for 10 minutes. 0.5 ml 10×PBS was added to neutralize the lysing buffer. After spinning at 1200 rpm for 10 minutes at room temperature, cells were washed with R10, re-centrifuged and re-suspended in 1 ml of R10 medium. To assess the viability of the cells, splenocytes were diluted 1:10 in Guava ViaCount Reagent and counted using an Accuri C6 FACS sorter. Cell viability was typically 96-99%. The provided peptide pools were resuspended in DMSO at 0.33 mg/ml each and were composed as follows—Core: 35 peptides, Pol1: 103 peptides, and Pol2: 105 peptides.

The ELISPOT assay was performed in accordance with standard practice using four plates wherein, for each sample, 400,000 or 200,000 splenocytes were plated in duplicate, and stimulated with DMSO, core or ConA.

The peptide pools were used at a final concentration of 1 μg/ml. The assay revealed a low background (DMSO stimulation) and activation with Concanavalin A (ConA). The Spot Forming Cells (SFC) were calculated with the following formula: [(2.5*average400K)+(5*average200K)]/2. Quality check and data elaboration showed an absent immune response in all animals, including those vaccinated with pCore at day 0.

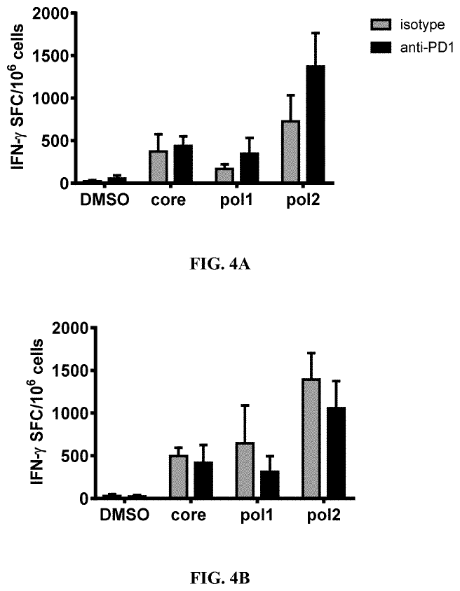

The results are shown in FIG. 4 and indicate a strong immune response against Core and Pol antigens, consistent with data obtained in previous vaccination studies. No significant background (DMSO) was measured, whereas a strong response was measured against the Pol2 peptide pool. A lower response was measured against the Pol1 peptide pool. Interestingly, the group receiving three injections of anti-PD-1 showed a significantly higher response against Pol antigens (p=0.04 and p=0.015 for Pol1 and Pol2, respectively) compared to the Isotype antibody. In addition, the response against Pol in groups receiving three anti-PD-1 injections was also higher compared to mice receiving four antibody treatments (p=0.02 and p=0.004 for Pol1 and Pol2, respectively). Taken together, these data suggest that 3× anti-PD-1 administration can increase HBV-specific immune response in vaccinated healthy mice.

To further characterize the immune response, splenocytes were analyzed by multicolor ICS for the secretion of IFNγ, TNFα and IL2. The assay was executed in accordance with standard ICS practice.

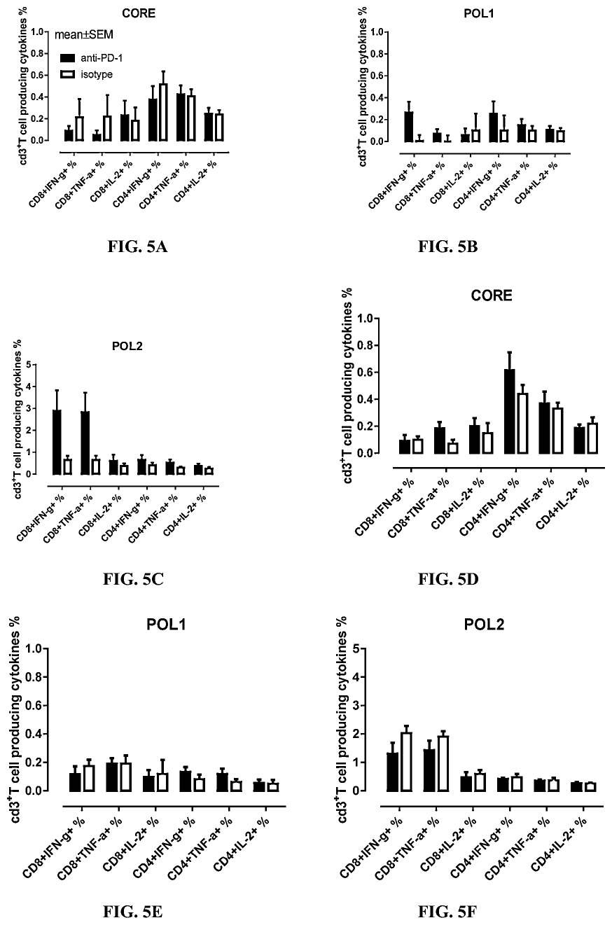

The results are shown in FIG. 5.

The ICS analysis was in line with the ELISPOT findings: Core-specific response was mainly CD4+/IFNγ+/TNFα+/IL2+, whereas Pol1-specific was both CD4+ and CD8+/IFNγ+/TNFα+/IL2+. In contrast, Pol2-specific CMI was CD8+/IFNγ+/TNFα+. Pol-specific CMI detected by ICS was higher in mice treated 3 times with anti-PD-1 compared to the other groups.

Taken together, these data show that the HBV Core/Pol vaccine is immunogenic and that 3 administrations of anti-PD-1 can result in optimal immune effects.

### Example 5. Impact of Anti-PD-1 on HBV Vaccine-Induced Immune Response in AAV/HBV Infected Mice

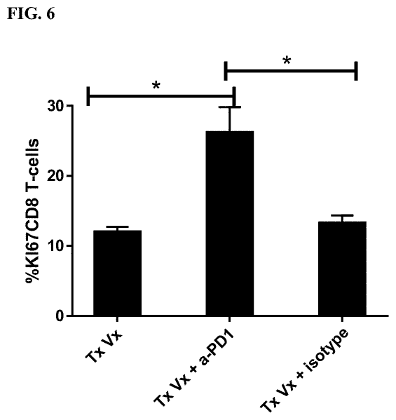

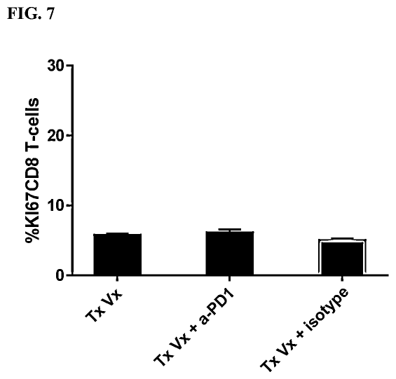

To further assess whether blocking PD-1 impacts HBV-vaccine induced cell-mediated immunity (CMI) in AAV/HBV infected mice, adult C57BL/6 mice were infected with AAV/HBV for 28 days. Afterwards one group of 14 animals was vaccinated by DNA electroporation (2.5 μg of each plasmid) at d28 and d49, one group was vaccinated by DNA electroporation at d28 and d49 and was treated with anti-PD-1 antibody (10 mg/kg) at day 56. The third group of animals was treated with the isotype antibody after vaccination. At day 63, mice were sacrificed. Splenocytes were isolated as well as intrahepatic lymphocytes (IHL), and proliferative capacity of CD8 T-cells was measured. As shown in FIG. 6, IHL from mice treated with anti-PD-1 had significantly higher CD8 proliferative T-cells compared to those not treated with anti-PD-1 and those treated with the isotope antibody. In contrast, splenocytes did not show higher proliferative capacity (FIG. 7), which reflects the fact that proliferative CD8 T-cells migrate to the sites were the infection take place in this case the liver.

Animals: C57BL/6 mice were infected with rAAV8-1.3HBV (Fiveplus Medical Institute, China) via the tail vein at MOI 10¹¹. Vaccination was performed when HBV chronicity was reached 28 days after infection.

Plasmid DNA: The DNA vaccine was a combination of two separate DNA plasmids encoding the Core (pDF-Core) and Polymerase (Pol) proteins (pDF-Pol) of HBV, as shown in FIG. 1A and FIG. 1B, respectively. 2.5 μg of each plasmid was electroporated with trigrid system i.m.

Antibodies: Anti-PD-1 antibody (RMP1-14, Bioxcell), isotype control antibody (Bioxcell). 10 μg/ml of the antibody was used in the study, administered i.p.

Study design: Adult C57BL/6 mice were infected with AAV/HBV for 28 days. Afterwards, one group of 14 animals was vaccinated by DNA electroporation (2.5 μg of each plasmid) at d28 and d49, one group was vaccinated by DNA electroporation at d28 and d49 and were treated with anti-PD-1 antibody (10 mg/kg) at day 56. The third group of animals was treated with the isotype antibody after vaccination.

Isolation of splenocytes and IHL: Spleens were isolated from mice; tissue disruption was performed using GentleMACS Dissociater (Miltenyi Biotec), and cells were counted and used in the designated assays. The intrahepatic lymphocytes were obtained by perfusing the liver with PBS, tissue disruption was performed using GentleMACS Dissociater, followed by a 33.75% (v/v) Percoll®/PBS2% FCS gradient. Centrifugation at 700×g (12 minutes, room temperature, max acceleration and brake at 1) separated the hepatocytes from the lymphocytes. The samples were aspirated carefully and used in designated assay.

Immunophenotyping: Splenocytes and IHL ware plated 100.000 cells per well. Next they were stained with viability dye. Afterwards cells were fixed and permeabilized to perform intracellular staining. Cells were stained with following markers: anti-CD3, anti-CD8, anti-CD4, and ki67, which is a marker for proliferative T-cells. After 1 hour, cells were washed twice, resuspended in 100 μl stain buffer and read-out was done on Facs fortessa.

It is understood that the examples and embodiments described herein are for illustrative purposes only, and that changes could be made to the embodiments described above without departing from the broad inventive concept thereof. It is understood, therefore, that this invention is not limited to the particular embodiments disclosed, but it is intended to cover modifications within the spirit and scope of the invention as defined by the appended claims.

1-16. (canceled)

17. A therapeutic combination for treating a hepatitis B virus (HBV) infection in a subject in need thereof, comprising:
i) a non-naturally occurring polynucleotide sequence encoding a HBV polymerase antigen consisting of an amino acid sequence that is at least 90% identical to SEQ ID NO: 7, wherein the HBV polymerase antigen does not have reverse transcriptase activity and RNase H activity and is capable of inducing a T cell response against at least HBV genotypes B, C and D; and
ii) an anti-PD-1 or anti-PD-L1 antibody or antigen-binding fragment thereof.

18. The therapeutic combination of claim 17, further comprising a non-naturally occurring polynucleotide sequence encoding a truncated HBV core antigen consisting of an amino acid sequence that is at least 95% identical to SEQ ID NO: 2.

19. A therapeutic combination for treating a hepatitis B virus (HBV) infection in a subject in need thereof, comprising:
i) a non-naturally occurring polynucleotide sequence encoding a HBV polymerase antigen consisting of an amino acid sequence that is at least 90% identical to SEQ ID NO: 7, wherein the HBV polymerase antigen does not have reverse transcriptase activity and RNase H activity and is capable of inducing a T cell response against at least HBV genotypes B, C and D; and
ii) an anti-PD-1 or anti-PD-L1 antibody or antigen-binding fragment thereof selected from the group consisting of:
a) 17D8, 2D3, 4H1, 5C4, 4A11, 7D3, 5F4, H409A11, hPD-1.08A, hPD-1.09A, 109A-H, K09A-L-11, K09A-L-16, K09A-L-17, 409A-H, H1M7789N, H1M7799N, H1M7800N, H2M7780N, H2M7788N, H2M7790N, H2M7791N, H2M7794N, H2M7795N, H2M7796N, H2M7798N, H4H9019P, H4xH9034P2, H4xH9035P2, H4xH9037P2, H4xH9045P2, H4xH9048P2, H4H9057P2, H4H9068P2, H4xH9119P2, H4xH9120P2, H4xH9128P2, H4xH9135P2, H4xH9145P2, H4xH8992P, H4xH8999P, H4xH9008P, H4H7798N, H4H7795N2, H4H9008P, H4H9048P2, PD1-0103, PD1-0098, PD 1-0050, PD 1-0069, PD1-0073, PD1-0078, PD1-0102, PD1-0103_01, PD1-0103_02, PD1-0103_03, PD1-0103_04, PD1-0103-0312, PD1-0103-0313, PD1-0103-0314, PD1-0103-0315, 1.7.3 hAb, 1.49.9 hAb, 1.103.11 hAb, 1.103.11-v2 hAb, 1.139.15 hAb, 1.153.7 hAb, or a variant thereof,
b) an antibody binding to an epitope having a sequence of SEHSI (SEQ ID NO: 25), DPFEL (SEQ ID NO: 26), KLNG (SEQ ID NO: 27), QTSWK (SEQ ID NO: 28), LHFEP (SEQ ID NO: 29), NDNGSY (SEQ ID NO: 30), TTLYVT (SEQ ID NO: 31), or LAAFPEDRSQPGQDCR (SEQ ID NO: 32); and
c) Nivolumab, Pembrolizumab, TSR-042, REGN2810, EH12.2H7, Avelumab, Durvalumab, Cemiplimab, BMS-936559, Atezolizumab, or an equivalent thereto.

20. The therapeutic combination of claim 19, further comprising a non-naturally occurring polynucleotide sequence encoding a truncated HBV core antigen consisting of an amino acid sequence that is at least 95% identical to SEQ ID NO: 2.

21. The therapeutic combination of claim 17, further comprising a polynucleotide sequence encoding a signal sequence operably linked to the N-terminus of the HBV polymerase antigen.

22. The therapeutic combination of claim 18, wherein:
a) the truncated HBV core antigen consists of the amino acid sequence of SEQ ID NO: 2 or SEQ ID NO: 4; and
b) the HBV polymerase antigen comprises the amino acid sequence of SEQ ID NO: 7.

23. The therapeutic combination of claim 17, wherein the non-naturally occurring polynucleotide sequence is a DNA sequence.

24. The therapeutic combination of claim 18, comprising a non-naturally occurring nucleic acid molecule encoding both the HBV polymerase antigen and the HBV core antigen.

25. The therapeutic combination of claim 18, comprising a first non-naturally occurring nucleic acid molecule encoding the HBV polymerase antigen and a second, different non-naturally occurring nucleic acid molecule encoding the HBV core antigen.

26. The therapeutic combination of claim 18, wherein the polynucleotide sequence encoding the HBV core antigen comprises a polynucleotide sequence having at least 90% sequence identity to SEQ ID NO: 1 or SEQ ID NO: 3.

27. The therapeutic combination of claim 26, wherein the polynucleotide sequence encoding the HBV core antigen comprises the polynucleotide sequence of SEQ ID NO: 1 or SEQ ID NO: 3.

28. The therapeutic combination of claim 17, wherein the polynucleotide sequence encoding the HBV polymerase antigen comprises a polynucleotide sequence having at least 90% sequence identity to SEQ ID NO: 5 or SEQ ID NO: 6.

29. The therapeutic combination of claim 28, wherein the polynucleotide sequence encoding the HBV polymerase antigen comprises the polynucleotide sequence of SEQ ID NO: 5 or SEQ ID NO: 6.

30. The therapeutic combination of claim 17, wherein the anti-PD-1 or anti-PD-L1 antibody or antigen-binding fragment thereof is Nivolumab, Pembrohzumab, TSR-042, REGN2810, EH12.2H7, Avelumab, Durvalumab, Cemiplimab, BMS-936559, or Atezolizumab, or an equivalent thereto.

31. The therapeutic combination of claim 17, wherein the non-naturally occurring polynucleotide sequence encodes the HBV polymerase antigen consisting of an amino acid sequence that is at least 98% identical to SEQ ID NO: 7.

32. A therapeutic combination for treating a hepatitis B virus (HBV) infection in a subject in need thereof, comprising:
i) a non-naturally occurring polynucleotide sequence encoding a HBV polymerase antigen consisting of an amino acid sequence that is at least 98% identical to SEQ ID NO: 7, wherein the HBV polymerase antigen does not have reverse transcriptase activity and RNase H activity and is capable of inducing a T cell response against at least HBV genotypes B, C and D; and
ii) an anti-PD-1 or anti-PD-L1 antibody or antigen-binding fragment thereof selected from the group consisting of:
a) 17D8, 2D3, 4H1, 5C4, 4A11, 7D3, 5F4, H409A11, hPD-1.08A, hPD-1.09A, 109A-H, K09A-L-11, K09A-L-16, K09A-L-17, 409A-H, H1M7789N, H1M7799N, H1M7800N, H2M7780N, H2M7788N, H2M7790N, H2M7791N, H2M7794N, H2M7795N, H2M7796N, H2M7798N, H4H9019P, H4xH9034P2, H4xH9035P2, H4xH9037P2, H4xH9045P2, H4xH9048P2, H4H9057P2, H4H9068P2, H4xH9119P2, H4xH9120P2, H4xH9128P2, H4xH9135P2, H4xH9145P2, H4xH8992P, H4xH8999P, H4xH9008P, H4H7798N, H4H7795N2, H4H9008P, H4H9048P2, PD1-0103, PD1-0098, PD 1-0050, PD 1-0069, PD1-0073, PD1-0078, PD1-0102, PD1-0103_01, PD1-0103_02, PD1-0103_03, PD1-0103_04, PD1-0103-0312, PD1-0103-0313, PD1-0103-0314, PD1-0103-0315, 1.7.3 hAb, 1.49.9 hAb, 1.103.11 hAb, 1.103.11-v2 hAb, 1.139.15 hAb, 1.153.7 hAb, or a variant thereof,
b) an antibody binding to an epitope having a sequence of SEHSI (SEQ ID NO: 25), DPFEL (SEQ ID NO: 26), KLNG (SEQ ID NO: 27), QTSWK (SEQ ID NO: 28), LHFEP (SEQ ID NO: 29), NDNGSY (SEQ ID NO: 30), TTLYVT (SEQ ID NO: 31), or LAAFPEDRSQPGQDCR (SEQ ID NO: 32); and
c) Nivolumab, Pembrolizumab, TSR-042, REGN2810, EH12.2H7, Avelumab, Durvalumab, Cemiplimab, BMS-936559, Atezolizumab, or an equivalent thereto.

33. The therapeutic combination of claim 32, further comprising a non-naturally occurring polynucleotide sequence encoding a truncated HBV core antigen consisting of an amino acid sequence that is at least 95% identical to SEQ ID NO 2.

34. A method of treating a hepatitis B virus (HBV) infection or an HBV-induced disease in a subject in need thereof, comprising administering to the subject the therapeutic combination of claim 17.


SEQUENCE LISTING (32 sequences)

SEQ ID NO: 1 - DNA, 444 bp, Artificial Sequence
  HBV truncated core antigen gene
gacatcgacccttacaaggagttcggcgccagcgtggaactgctgtcttttctgcccagtgatttctttccttccattcgagacctgctggataccgcctctgctctgtatcgggaagccctggagagcccagaacactgctccccacaccataccgctctgcgacaggcaatcctgtgctggggggagctgatgaacctggccacatgggtgggatcgaatctggaggaccccgcttcacgggaactggtggtcagctacgtgaacgtcaatatgggcctgaaaatccgccagctgctgtggttccatattagctgcctgacttttggacgagagaccgtgctggaatacctggtgtccttcggcgtctggattcgcactccccctgcttatcgaccacccaacgcaccaattctgtccaccctgcccgagaccacagtggtc

SEQ ID NO: 2 - protein, 148 aa, Artificial Sequence
  HBV truncated core antigen
DIDPYKEFGASVELLSFLPSDFFPSIRDLLDTASALYREALESPEHCSPHHTALRQAILCWGELMNLATWVGSNLEDPASRELVVSYVNVNMGLKIRQLLWFHISCLTFGRETVLEYLVSFGVWIRTPPAYRPPNAPILSTLPETTVV

SEQ ID NO: 3 - DNA, 447 bp, Artificial Sequence
  HBV truncated core antigen gene
atggacatcgacccttacaaggagttcggcgccagcgtggaactgctgtcttttctgcccagtgatttctttccttccattcgagacctgctggataccgcctctgctctgtatcgggaagccctggagagcccagaacactgctccccacaccataccgctctgcgacaggcaatcctgtgctggggggagctgatgaacctggccacatgggtgggatccaatctggaggaccccgcttcacgggaactggtggtcagctacgtgaacgtcaatatgggcctgaaaatccgccagctgctgtggttccatattagctgcctgacttttggacgagagaccgtgctggaatacctggtgtccttcggcgtctggatccgcactccccctgcttatcgaccacccaacgcaccaattctgtccaccctgcccgagaccacagtggtc

SEQ ID NO: 4 - protein, 149 aa, Artificial Sequence
  HBV truncated core antigen
MDIDPYKEFGASVELLSFLPSDFFPSIRDLLDTASALYREALESPEHCSPHHTALRQAILCWGELMNLATWVGSNLEDPASRELVVSYVNVNMGLKIRQLLWFHISCLTFGRETVLEYLVSFGVWIRTPPAYRPPNAPILSTLPETTVV

SEQ ID NO: 5 - DNA, 2529 bp, Artificial Sequence
  HBV pol antigen gene
atgcccctgtcttaccagcactttagaaagcttctgctgctggacgatgaagccgggcctctggaggaagagctgccaaggctggcagacgaggggctgaaccggagagtggccgaagatctgaatctgggaaacctgaacgtgagcatcccttggactcataaagtcggcaacttcaccgggctgtacagctccacagtgcctgtcttcaatccagagtggcagacaccatcctttcccaacattcacctgcaggaggacatcattaatagatgcgaacagttcgtgggacctctgacagtcaacgaaaagaggcgcctgaaactgatcatgcctgccaggttttacccaaatgtgactaagtatctgccactggataagggcatcaagccttactatccagagcacctggtgaaccattacttccagactagacactatctgcataccctgtggaaggccggaatcctgtacaaacgagaaactacccggagtgcttcattttgtggctccccatattcttgggaacaggagctgcagcatggcaggctggtgttccagaccagcacacgccacggggatgagtccttttgccagcagtctagtggcatcctgagcagatcccccgtggggccttgtctgcagtctcagctgcggaagagtagactgggactgcagccacagcagggacacctggcacgacggcagcagggaaggtctggcagtatccgggctagagtgcatcccacaactagaaggcctttcggcgtcgagccatcaggaagcggccacaccacaaacaccgcatcaagctcctctagttgcctgcatcagtcagccgtgagaaaggccgcttacagccacctgtccacatctaaaaggcactcaagctccgggcatgctgtggagctgcacaacatccctccaaattctgcacgcagtcagtcagaaggacccgtgttcagctgctggtggctgcagtttcggaactcaaagccttgcagcgactattgtctgagccatattgtgaatctgctggaggattggggcccttgtaccgagcacggggaacaccatatcaggattccacgaacaccagcacgagtgactggaggggtgttcctggtggacaagaacccccacaatactaccgagagccggctggtggtcgatttcagtcagttttcaagaggcaacacaagggtgtcatggcccaaattcgccgtccctaatctgcagagtctgactaacctgctgtctagtaatctgagctggctgtccctggacgtgtccgcagccttttaccacctgcctctgcatccagctgcaatgccccatctgctggtggggtcaagcggactgagtcgctacgtcgcccgactgtcctctaactcacgcatcattaatcaccagcatggcaccatgcagaacctgcacgatagctgttcccggaatctgtacgtgtctctgctgctgctgtataagacattcggcagaaaactgcacctgtacagccatcctatcattctggggtttaggaagatcccaatgggagtgggactgagccccttcctgctggcacagtttacctccgccatttgctctgtggtccgccgagccttcccacactgtctggctttttcctatatgaacaatgtggtcctgggcgccaaatccgtgcagcatctggagtctctgttcacagctgtcactaactttctgctgagcctggggatccacctgaacccaaataagactaaacgctgggggtacagcctgaatttcatgggatatgtgattggatcctgggggaccctgccacaggagcacatcgtgcagaagatcaaggaatgctttcggaagctgcccgtcaacagacctatcgactggaaagtgtgccagcggattgtcggactgctgggcttcgccgctccctttacccagtgcgggtacccagcactgatgcccctgtatgcctgtatccagtctaagcaggctttcacctttagtcctacatacaaggcattcctgtgcaaacagtacctgaacctgtatccagtggcaaggcagcgacctggactgtgccaggtctttgcaaatgccactcctaccggctgggggctggctatcggacatcagcgaatgcggggcacattcgtggcccccctgcctattcacactgctcagctgctggcagcctgctttgctagatctaggagtggagcaaagctgatcggcaccgacaatagtgtggtcctgtcaagaaaatacacatccttcccatggctgctgggatgtgctgcaaactggattctgaggggcaccagcttcgtgtacgtcccctcagccctgaatcctgctgacgatccatcccgcgggcgactgggactgtaccgacctctgctgagactgcccttcaggcctacaactggccggacatctctgtatgccgattcaccaagcgtgccctcacacctgcctgacagagtccactttgcttcacccctgcacgtcgcttggcggcctcca

SEQ ID NO: 6 - DNA, 2529 bp, Artificial Sequence
  HBV pol antigen gene
atgcccctgtcttaccagcactttagaaagctgctgctgctggacgatgaagccgggcctctggaggaagagctgccaaggctggcagacgaggggctgaaccggagagtggccgaagatctgaatctgggaaacctgaacgtgagcatcccttggactcataaagtcggcaacttcaccgggctgtacagctccacagtgcctgtcttcaatccagagtggcagacaccatcctttcccaacattcacctgcaggaggacatcattaatagatgcgaacagttcgtgggacctctgacagtcaacgaaaagaggcgcctgaaactgatcatgcctgccaggttttacccaaatgtgactaagtatctgccactggataagggcatcaagccttactatccagagcacctggtgaaccattacttccagactagacactatctgcataccctgtggaaggccggaatcctgtacaaacgagaaactacccggagtgcttcattttgtggctccccatattcttgggaacaggagctgcagcatggcaggctggtgttccagaccagcacacgccacggggatgagtccttttgccagcagtctagtggcatcctgagcagatcccccgtggggccttgtctgcagtctcagctgcggaagagtagactgggactgcagccacagcagggacacctggcacgacggcagcagggaaggtctggcagtatccgggctagagtgcatcccacaactagaaggcctttcggcgtcgagccatcaggaagcggccacaccacaaacaccgcatcaagctcctctagttgcctgcatcagtcagccgtgagaaaggccgcttacagccacctgtccacatctaaaaggcactcaagctccgggcatgctgtggagctgcacaacatccctccaaattctgcacgcagtcagtcagaaggacccgtgttcagctgctggtggctgcagtttcggaactcaaagccttgcagcgactattgtctgagccatattgtgaatctgctggaggattggggcccttgtaccgagcacggggaacaccatatcaggattccacgaacaccagcacgagtgactggaggggtgttcctggtggacaagaacccccacaatactaccgagagccggctggtggtcgatttcagtcagttttcaagaggcaacacaagggtgtcatggcccaaattcgccgtccctaatctgcagagtctgactaacctgctgtctagtaatctgagctggctgtccctggacgtgtccgcagccttttaccacctgcctctgcatccagctgcaatgccccatctgctggtggggtcaagcggactgagtcgctacgtcgcccgactgtcctctaactcacgcatcattaatcaccagcatggcaccatgcagaacctgcacgatagctgttcccggaatctgtacgtgtctctgctgctgctgtataagacattcggcagaaaactgcacctgtacagccatcctatcattctggggtttaggaagatcccaatgggagtgggactgagccccttcctgctggcacagtttacctccgccatttgctctgtggtccgccgagccttcccacactgtctggctttttcctatatgaacaatgtggtcctgggcgccaaatccgtgcagcatctggagtctctgttcacagctgtcactaactttctgctgagcctggggatccacctgaacccaaataagactaaacgctgggggtacagcctgaatttcatgggatatgtgattggatcctgggggaccctgccacaggagcacatcgtgcagaagatcaaggaatgctttcggaagctgcccgtcaacagacctatcgactggaaagtgtgccagcggattgtcggactgctgggcttcgccgctccctttacccagtgcgggtacccagcactgatgcccctgtatgcctgtatccagtctaagcaggctttcacctttagtcctacatacaaggcattcctgtgcaaacagtacctgaacctgtatccagtggcaaggcagcgacctggactgtgccaggtctttgcaaatgccactcctaccggctgggggctggctatcggacatcagcgaatgcggggcacattcgtggcccccctgcctattcacactgctcagctgctggcagcctgctttgctagatctaggagtggagcaaagctgatcggcaccgacaatagtgtggtcctgtcaagaaaatacacatccttcccatggctgctgggatgtgctgcaaactggattctgaggggcaccagcttcgtgtacgtcccctcagccctgaatcctgctgacgatccatcccgcgggcgactgggactgtaccgacctctgctgagactgcccttcaggcctacaactggccggacatctctgtatgccgattcaccaagcgtgccctcacacctgcctgacagagtccactttgcttcacccctgcacgtcgcttggcggcctcca

SEQ ID NO: 7 - protein, 843 aa, Artificial Sequence
  HBV pol antigen
MPLSYQHFRKLLLLDDEAGPLEEELPRLADEGLNRRVAEDLNLGNLNVSIPWTHKVGNFTGLYSSTVPVFNPEWQTPSFPNIHLQEDIINRCEQFVGPLTVNEKRRLKLIMPARFYPNVTKYLPLDKGIKPYYPEHLVNHYFQTRHYLHTLWKAGILYKRETTRSASFCGSPYSWEQELQHGRLVFQTSTRHGDESFCQQSSGILSRSPVGPCLQSQLRKSRLGLQPQQGHLARRQQGRSGSIRARVHPTTRRPFGVEPSGSGHTTNTASSSSSCLHQSAVRKAAYSHLSTSKRHSSSGHAVELHNIPPNSARSQSEGPVFSCWWLQFRNSKPCSDYCLSHIVNLLEDWGPCTEHGEHHIRIPRTPARVTGGVFLVDKNPHNTTESRLVVDFSQFSRGNTRVSWPKFAVPNLQSLTNLLSSNLSWLSLDVSAAFYHLPLHPAAMPHLLVGSSGLSRYVARLSSNSRIINHQHGTMQNLHDSCSRNLYVSLLLLYKTFGRKLHLYSHPIILGFRKIPMGVGLSPFLLAQFTSAICSVVRRAFPHCLAFSYMNNVVLGAKSVQHLESLFTAVTNFLLSLGIHLNPNKTKRWGYSLNFMGYVIGSWGTLPQEHIVQKIKECFRKLPVNRPIDWKVCQRIVGLLGFAAPFTQCGYPALMPLYACIQSKQAFTFSPTYKAFLCKQYLNLYPVARQRPGLCQVFANATPTGWGLAIGHQRMRGTFVAPLPIHTAQLLAACFARSRSGAKLIGTDNSVVLSRKYTSFPWLLGCAANWILRGTSFVYVPSALNPADDPSRGRLGLYRPLLRLPFRPTTGRTSLYADSPSVPSHLPDRVHFASPLHVAWRPP

SEQ ID NO: 8 - DNA, 63 bp, Artificial Sequence
  Cystatin S signal peptide coding sequence
atggctcgacctctgtgtaccctgctactcctgatggctaccctggctggagctctggccagc

SEQ ID NO: 9 - protein, 21 aa, Artificial Sequence
  Cystatin S signal peptide sequence
MARPLCTLLLLMATLAGALAS

SEQ ID NO: 10 - DNA, 378 bp, Artificial Sequence
  triple enhancer regulatory sequence
ggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctagttctctcgttaacttaatgagacagatagaaactggtcttgtagaaacagagtagtcgcctgcttttctgccaggtgctgacttctctcccctgggcttttttctttttctcaggttgaaaagaagaagacgaagaagacgaagaagac

SEQ ID NO: 11 - DNA, 12 bp, Artificial Sequence
  linker coding sequence
gccggagctggc

SEQ ID NO: 12 - DNA, 248 bp, Artificial Sequence
  ApoAI gene fragment
ttggccgtgctcttcctgacgggtaggtgtcccctaacctagggagccaaccatcggggggccttctccctaaatccccgtggcccaccctcctgggcagaggcagcaggtttctcactggccccctctcccccacctccaagcttggcctttcggctcagatctcagcccacagctggcctgatctgggtctcccctcccaccctcagggagccaggctcggcatttcgtcgacaagcttagccacc

SEQ ID NO: 13 - DNA, 130 bp, Artificial Sequence
  SV40 polyadenylation signal sequence
aacttgtttattgcagcttataatggttacaaataaagcaatagcatcacaaatttcacaaataaagcatttttttcactgcattctagttgtggtttgtccaaactcatcaatgtatcttatcatgtct

SEQ ID NO: 14 - DNA, 81 bp, Artificial Sequence
  immunoglobulin secretion signal coding sequence
atggagttcggcctgtcttgggtctttctggtggcaatcctgaagggcgtgcagtgtgaagtgcagctgctggagtctgga

SEQ ID NO: 15 - protein, 27 aa, Artificial Sequence
  immunoglobulin secretion signal sequence
MEFGLSWVFLVAILKGVQCEVQLLESG

SEQ ID NO: 16 - protein, 996 aa, Artificial Sequence
  HBV core-pol fusion antigen sequence
MDIDPYKEFGASVELLSFLPSDFFPSIRDLLDTASALYREALESPEHCSPHHTALRQAILCWGELMNLATWVGSNLEDPASRELVVSYVNVNMGLKIRQLLWFHISCLTFGRETVLEYLVSFGVWIRTPPAYRPPNAPILSTLPETTVVAGAGMPLSYQHFRKLLLLDDEAGPLEEELPRLADEGLNRRVAEDLNLGNLNVSIPWTHKVGNFTGLYSSTVPVFNPEWQTPSFPNIHLQEDIINRCEQFVGPLTVNEKRRLKLIMPARFYPNVTKYLPLDKGIKPYYPEHLVNHYFQTRHYLHTLWKAGILYKRETTRSASFCGSPYSWEQELQHGRLVFQTSTRHGDESFCQQSSGILSRSPVGPCLQSQLRKSRLGLQPQQGHLARRQQGRSGSIRARVHPTTRRPFGVEPSGSGHTTNTASSSSSCLHQSAVRKAAYSHLSTSKRHSSSGHAVELHNIPPNSARSQSEGPVFSCWWLQFRNSKPCSDYCLSHIVNLLEDWGPCTEHGEHHIRIPRTPARVTGGVFLVDKNPHNTTESRLVVDFSQFSRGNTRVSWPKFAVPNLQSLTNLLSSNLSWLSLDVSAAFYHLPLHPAAMPHLLVGSSGLSRYVARLSSNSRIINHQHGTMQNLHDSCSRNLYVSLLLLYKTFGRKLHLYSHPIILGFRKIPMGVGLSPFLLAQFTSAICSVVRRAFPHCLAFSYMNNVVLGAKSVQHLESLFTAVTNFLLSLGIHLNPNKTKRWGYSLNFMGYVIGSWGTLPQEHIVQKIKECFRKLPVNRPIDWKVCQRIVGLLGFAAPFTQCGYPALMPLYACIQSKQAFTFSPTYKAFLCKQYLNLYPVARQRPGLCQVFANATPTGWGLAIGHQRMRGTFVAPLPIHTAQLLAACFARSRSGAKLIGTDNSVVLSRKYTSFPWLLGCAANWILRGTSFVYVPSALNPADDPSRGRLGLYRPLLRLPFRPTTGRTSLYADSPSVPSHLPDRVHFASPLHVAWRPP

SEQ ID NO: 17 - protein, 1023 aa, Artificial Sequence
  HBV core-pol fusion antigen sequence with Ig signal sequence
MEFGLSWVFLVAILKGVQCEVQLLESGMDIDPYKEFGASVELLSFLPSDFFPSIRDLLDTASALYREALESPEHCSPHHTALRQAILCWGELMNLATWVGSNLEDPASRELVVSYVNVNMGLKIRQLLWFHISCLTFGRETVLEYLVSFGVWIRTPPAYRPPNAPILSTLPETTVVAGAGMPLSYQHFRKLLLLDDEAGPLEEELPRLADEGLNRRVAEDLNLGNLNVSIPWTHKVGNFTGLYSSTVPVFNPEWQTPSFPNIHLQEDIINRCEQFVGPLTVNEKRRLKLIMPARFYPNVTKYLPLDKGIKPYYPEHLVNHYFQTRHYLHTLWKAGILYKRETTRSASFCGSPYSWEQELQHGRLVFQTSTRHGDESFCQQSSGILSRSPVGPCLQSQLRKSRLGLQPQQGHLARRQQGRSGSIRARVHPTTRRPFGVEPSGSGHTTNTASSSSSCLHQSAVRKAAYSHLSTSKRHSSSGHAVELHNIPPNSARSQSEGPVFSCWWLQFRNSKPCSDYCLSHIVNLLEDWGPCTEHGEHHIRIPRTPARVTGGVFLVDKNPHNTTESRLVVDFSQFSRGNTRVSWPKFAVPNLQSLTNLLSSNLSWLSLDVSAAFYHLPLHPAAMPHLLVGSSGLSRYVARLSSNSRIINHQHGTMQNLHDSCSRNLYVSLLLLYKTFGRKLHLYSHPIILGFRKIPMGVGLSPFLLAQFTSAICSVVRRAFPHCLAFSYMNNVVLGAKSVQHLESLFTAVTNFLLSLGIHLNPNKTKRWGYSLNFMGYVIGSWGTLPQEHIVQKIKECFRKLPVNRPIDWKVCQRIVGLLGFAAPFTQCGYPALMPLYACIQSKQAFTFSPTYKAFLCKQYLNLYPVARQRPGLCQVFANATPTGWGLAIGHQRMRGTFVAPLPIHTAQLLAACFARSRSGAKLIGTDNSVVLSRKYTSFPWLLGCAANWILRGTSFVYVPSALNPADDPSRGRLGLYRPLLRLPFRPTTGRTSLYADSPSVPSHLPDRVHFASPLHVAWRPP

SEQ ID NO: 18 - DNA, 584 bp, Artificial Sequence
  hCMV promoter
tgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagc

SEQ ID NO: 19 - DNA, 684 bp, Artificial Sequence
  hCMV promoter sequence
accgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcgg

SEQ ID NO: 20 - DNA, 225 bp, Artificial Sequence
  bGH polyA signal
ctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgg

SEQ ID NO: 21 - DNA, 671 bp, Artificial Sequence
  pUC ORI
cccgtagaaaagatcaaaggatcttcttgagatcctttttttctgcgcgtaatctgctgcttgcaaacaaaaaaaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacat

SEQ ID NO: 22 - DNA, 795 bp, Artificial Sequence
  Kanr
atgattgagcaagatggtcttcacgctggctcgccagctgcgtgggtggaacgcctgtttggttatgattgggcgcagcagactattggatgttccgacgcggctgtatttcggctgtctgctcagggtcgccccgtgctgtttgtgaagacggatttgtctggcgcattaaatgagttacaggacgaggcggctcgtctgagttggttggccaccaccggcgtgccctgcgccgcagtgctggatgtcgtgacagaagcaggccgcgattggctccttctcggcgaagtgccgggccaggacctgctcagcagccacttggcaccggcagaaaaagtttctatcatggccgacgccatgcgtcgtcttcacactctcgatccggccacgtgcccctttgaccaccaggccaagcatcgtattgaacgtgcgcgtactcggatggaagcaggtttagtagaccaggacgatttggatgaggaacatcaaggcctggccccggctgaactgtttgcgcgcttaaaagcgtcgatgccagatggcgaagatttggtagtcacccatggagatgcgtgtttgccaaacatcatggttgaaaatggccgcttctcaggctttattgactgtgggcgcctgggtgttgccgaccgctatcaagatattgcgctcgcaactcgtgacatcgctgaagagctgggcggagaatgggctgaccgtttcctggtactgtatggcattgcagcgcccgattcccaacgcatcgcattttatcgtctgctggatgagtttttctaa

SEQ ID NO: 23 - protein, 264 aa, Artificial Sequence
  Codon optimized Kanr
MIEQDGLHAGSPAAWVERLFGYDWAQQTIGCSDAAVFRLSAQGRPVLFVKTDLSGALNELQDEAARLSWLATTGVPCAAVLDVVTEAGRDWLLLGEVPGQDLLSSHLAPAEKVSIMADAMRRLHTLDPATCPFDHQAKHRIERARTRMEAGLVDQDDLDEEHQGLAPAELFARLKASMPDGEDLVVTHGDACLPNIMVENGRFSGFIDCGRLGVADRYQDIALATRDIAEELGGEWADRFLVLYGIAAPDSQRIAFYRLLDEFF

SEQ ID NO: 24 - DNA, 99 bp, Artificial Sequence
  bla promoter
acccctatttgtttatttttctaaatacattcaaatatgtatccgctcatgagacaataaccctgataaatgcttcaataatattgaaaaaggaagagt

SEQ ID NO: 25 - protein, 5 aa, Artificial Sequence
  synthetic peptide
SEHSI

SEQ ID NO: 26 - protein, 5 aa, Artificial Sequence
  synthetic peptide
DPFEL

SEQ ID NO: 27 - protein, 4 aa, Artificial Sequence
  synthetic peptide
KLNG

SEQ ID NO: 28 - protein, 5 aa, Artificial Sequence
  synthetic peptide
QTSWK

SEQ ID NO: 29 - protein, 5 aa, Artificial Sequence
  synthetic peptide
LHFEP

SEQ ID NO: 30 - protein, 6 aa, Artificial Sequence
  synthetic peptide
NDNGSY

SEQ ID NO: 31 - protein, 6 aa, Artificial Sequence
  synthetic peptide
TTLYVT

SEQ ID NO: 32 - protein, 16 aa, Artificial Sequence
  synthetic peptide
LAAFPEDRSQPGQDCR